(19) United States
(12) Patent Application Publication
(10) Pub. No.: US 2009/0286696 A1
(43) Pub. Date: Nov. 19, 2009

(54) Double-Stranded Olidonucleotides and Uses Therefor

(75) Inventors: Nicholas Edward Dixon, Austinmer NSW (AU); Patrick Marcel Schaeffer, Alice River Qld (AU); Mark Donald Mulcair, Wantirna South Vic (AU)

Correspondence Address: JONES DAY, 222 EAST 41ST ST, NEW YORK, NY, 10017, US

(73) Assignee: University of Wollongong

(21) Appl. No.: 11/883,695

(22) PCT Filed: Feb. 3, 2006
(86) PCT No.: PCT/AU06/00136

§ 371 Date: Jun. 19, 2008

Related U.S. Application Data
(60) Provisional application No. 60/650,015, filed on Feb. 4, 2005.

(51) Int. Cl. C40B 40/06; C07H 21/04; C07K 2/00; C07K 14/245; C12P 19/34; C12N 15/63

(52) U.S. Cl. 506/16; 536/23.1; 530/322; 530/395; 435/91.3; 435/320.1

(57) ABSTRACT

The present invention relates generally to the field of screening or diagnostic applications in which a target is required to be displayed for binding to another molecule, or interaction or reaction with another molecule. In particular, the present invention relates to the use of DNA/protein interactions to immobilize or present one or more biomolecules for screening purposes. The present invention more particularly relates to double-stranded oligonucleotides, wherein said oligonucleotide comprises a first strand and a second strand, wherein: (a) said first strand comprises the sequence: 5′-N_{C} R N_{D} G T T G T A A C N_{D} A-3′ (SEQ ID NO: 1) or an analogue or derivative of said sequence; and (b) said second strand comprises the sequence: 5′-T N_{D} G T T A C A A C N_{D} T N_{C}-3′ (SEQ ID NO: 2) or an analogue or derivative of said sequence wherein R is a purine, N_{C} and N_{D} are each a DNA or RNA residue or analogue thereof, N_{D} residues in said first strand and said second strand are sufficiently complementary to permit said N_{D} residues to be annealed in the double-stranded oligonucleotide, and the sequence 5′-GTTGTAAC-3′ (SEQ ID NO: 3) of said first strand is annealed to the complementary sequence 5′GTTACAAC-3′ (SEQ ID NO: 4) of said second strand.

### FIELD OF THE INVENTION

The present invention relates generally to the field of screening or diagnostic applications in which a target is required to be displayed for binding to another molecule, or interaction or reaction with another molecule. In particular, the present invention relates to the use of DNA/protein interactions to immobilize or present one or more biomolecules onto solid, semi-solid or gel-like surfaces or to otherwise present one or more biomolecules for screening purposes. The present invention is therefore useful for a wide array of applications, including but not limited to screening for molecules as potential pharmaceuticals and/or agrochemicals.

### BACKGROUND TO THE INVENTION

A vast number of new drug targets are now being identified using a combination of genomics, bioinformatics, genetics, and high-throughput (HTP) biochemistry. Genomics provides information on the genetic composition and the activity of an organism's genes. Bioinformatics uses computer algorithms to recognize and predict structural patterns in DNA and proteins, defining families of related genes and proteins. The information gained from the combination of these approaches is expected to boost the number of drug targets, usually proteins, from the current 500 to over 10,000 in the coming decade.

The number of biomolecules (e.g., RNA, DNA, DNA/RNA hybrid, protein, antibodies, glycans, etc) and chemical compounds (e.g., small inorganic or organic compounds) available for screening as drug leads (i.e., potential drugs) is also growing dramatically due to recent advances in the field of biotechnology and combinatorial chemistry, including the identification of new screening platforms, identification of new drug targets, and the production of large numbers of organic compounds through rapid parallel and automated synthesis.

These factors create an enormous demand in the pharmaceutical and agrochemical industries for improved screening processes. In addition to the goal of achieving high-throughput screening of compounds against targets to identify potential drug leads, there is a need in the art for highly specific lead compounds early in the drug discovery process.

Many current technological screening and diagnostic platforms require the presentation or display of a biomolecule of interest (i.e., the “target”) for interaction with a test compound, which may be a peptide, nucleic acid, antibody or small molecule. Such platforms include, for example, multiwell plate-based screening systems, microarray-based screening systems, bacterial display, phage display, retroviral display, covalent display, ribosome display, or RNA display.

Ribosome display is a cell-free system for the in vitro presentation of proteins and peptides from large libraries for screening applications or molecular evolution. In ribosome display, the translated protein remains connected to the ribosome and to its encoding mRNA; the resulting ternary complex is used for selection. Nascent polypeptides are coupled to their corresponding mRNA, by forming stable polypeptide-ribosome-mRNA (PRM) complexes. This coupling provides for the nascent polypeptide to be presented on the surface of the ribosome, thereby facilitating its subsequent assay by virtue of its affinity for a test compound. The nascent polypeptide can also be isolated together with the encoding mRNA by virtue of its affinity for a ligand, wherein the encoding mRNA is then reverse-transcribed and/or amplified as DNA for further manipulation. To display a nascent polypeptide, nucleic acid encoding it is cloned downstream of an appropriate promoter (e.g., bacteriophage T3 or T7 promoter) and a ribosome binding sequence, optionally including a translatable spacer nucleic acid (e.g., encoding amino acids 211-299 of gene III of filamentous phage M13 mp19) that stabilizes the expressed fusion protein within the ribosomal tunnel. Ribosome complexes are stabilized against dissociation from the peptide and/or its encoding mRNA by the addition of reagents such as, for example, magnesium acetate or chloramphenicol.

Ribosome display has a number of advantages over cell-based systems such as phage display. It can display very large libraries without the restriction of bacterial transformation. It is also suitable for generating toxic, proteolytically sensitive and unstable proteins, and allows the incorporation of modified amino acids at defined positions. In combination with polymerase chain reaction (PCR)-based methods, mutations can be introduced efficiently into a selected DNA pool in subsequent cycles, leading to continuous DNA diversification and protein selection (in vitro protein evolution). Both prokaryotic and eukaryotic ribosome display systems have been developed and each has its own distinctive features.

In ribosome inactivation display, nucleic acid encoding the nascent polypeptide is linked to nucleic acid encoding a first spacer sequence (e.g., a glycine/serine rich sequence) which, in turn, is linked to a nucleic acid that encodes a toxin (e.g., ricin A) capable of inactivating a ribosome. In use, the toxin stalls the ribosome on the translation complex without release of the mRNA or the encoded peptide. The nucleic acid encoding the toxin may be linked to another nucleic acid encoding a second spacer that functions as an anchor to occupy the tunnel of the ribosome. This second spacer allows the peptide and the toxin to correctly fold and become active. Ribosome inactivation display libraries are generally transcribed and translated in vitro, using a system such as the rabbit reticulocyte lysate system available from Promega.

In mRNA display, mRNA is translated and covalently bonded to the polypeptide it encodes using puromycin as an adaptor molecule. The covalent mRNA-protein adduct is purified from the ribosome and used for selection. For example, nucleic acid encoding a polypeptide target can be linked to a nucleic acid encoding a spacer sequence (e.g., a glycine/serine rich sequence) positioned upstream of a transcription terminator and transcribed in vitro using a commercially available system (e.g., the HeLaScribe Nuclear Extract in vitro Transcription System available from Promega). The mRNA is then covalently linked to a DNA oligonucleotide that is, in turn, covalently-linked to puromycin, (see e.g., Roberts and Szostak, Proc. Natl. Acad. Sci. USA, 94, 12297-12302, 1997). It is also known to covalently link the puromycin-linked oligonucleotide to a psoralen moiety, to facilitate photo-crosslinking of the oligonucleotide to the transcribed mRNA. The mRNA is then translated. However, when the ribosome reaches the junction of the mRNA and the oligonucleotide during translation, it stalls and the puromycin moiety enters the phosphotransferase site of the ribosome thereby terminating translation and covalently linking the mRNA to the polypeptide.

In covalent display, nucleic acid encoding a polypeptide target of interest is linked, preferably in the same reading frame, to nucleic acid encoding a protein that interacts with a recognition site within the DNA encoding it (e.g., E. coli bacteriophage P2A or equivalent proteins from phage 186, HP1 or PSP3). The fusion construct is transcribed and translated in vitro, using a system such as the rabbit reticulocyte lysate system available from Promega. The encoded P2A protein nicks the nucleic acid at its recognition site in the fusion construct and forms a covalent bond with it such that the nucleic acid becomes covalently linked to the P2A peptide on the ribosome.

For drug screening applications, each of the foregoing systems require the presentation of a functional biomolecule or chemical compound such that it is capable of being assayed, e.g., to determine a biochemical reaction kinetic, DNA/protein interaction, RNA/protein interaction, protein/protein interaction, nucleic acid hybridization (e.g., DNA/DNA, RNA/DNA or RNA/RNA), melting point (Tm), spectral data, enzyme activity, enzyme co-factor requirement, drug metabolite, concentration, or fluorescence. The efficiency of presentation is therefore important to such drug screening applications, and is commercially significant in view of the reliance of the pharmaceutical and agrochemical industries on discovering new drug targets and drug leads.

Accordingly, there is a need in the art to improve the efficiencies of target presentation for such screening applications.

### SUMMARY OF THE INVENTION

According to a first aspect of the present invention, there is provided a double-stranded oligonucleotide, wherein said oligonucleotide comprises a first strand and a second strand, wherein:

(a) said first strand comprises the sequence:

(SEQ ID NO: 1)

5′-N_{C} R N_{D} G T T G T A A C N_{D} A-3′

or an analogue or derivative of said sequence; and

(b) said second strand comprises the sequence:

(SEQ ID NO: 2)

5′-T N_{D} G T T A C A A C N_{D} T N_{C} C-3′

or an analogue or derivative of said sequence
wherein R is a purine, N_{C} and N_{D} are each a DNA or RNA residue or analogue thereof, N_{D} residues in said first strand and said second strand are sufficiently complementary to permit said N_{D} residues to be annealed in the double-stranded oligonucleotide, and the sequence 5′-GTTGTAAC-3′ (SEQ ID NO: 3) of said first strand is annealed to the complementary sequence 5′-GTTACAAC-3′ (SEQ ID NO: 4) of said second strand.

The double-stranded oligonucleotide may comprise at least one additional DNA or RNA residue or analogue thereof, at either or both the 5′- and 3′-ends of either or both the first and second strands.

The double-stranded oligonucleotide may be forked.

The analogue may comprise a methylated, iodinated, brominated or biotinylated residue.

The double-stranded oligonucleotide may be derivatized to include 5′- and/or 3′-insertions that do not adversely affect its ability to bind to a Tus protein. The insertions may include the addition of mRNA and/or DNA that is to be presented or displayed.

In a first embodiment of the first aspect:

(a) said first strand comprises the sequence:

(SEQ ID NO: 55)

5′-(N_{A})ₘ N_{E} N_{E} N_{B} N_{B} N_{C} R N_{D} G T T G T A A C N_{D} A
(N_{A})ₙ-3′

or an analogue or derivative of said sequence; and

(b) said second strand comprises the sequence:

(SEQ ID NO: 56)

5′-(N_{A})ₚ T N_{D} G T T A C A A C N_{D} T N_{C} C N_{B} N_{E} N_{E}
(N_{A})ₒ-3′

or an analogue or derivative of said sequence

wherein N_{A}, N_{B} and N_{E} are each any DNA or RNA residue or analogue thereof, each of N_{A} and N_{B} is optional subject to the proviso that when any occurrence of N_{B} is present it is not base-paired to another residue, base-pairing of each of N_{C} to another residue is optional, each of N_{D} is base-paired with another residue, each of N_{E} is optional, subject to the proviso that if one or more of N_{E} is present it is not base-paired unless m=0 or o=0, m, n, o, p, are each an integer including zero, and said first strand and said second strand are to of equal or unequal length.

In a second embodiment of the first aspect, said first strand comprises the sequence:

(SEQ ID NO: 57)

5′-(N_{A})₁₋₁₅ N_{E} N_{E} N_{B} N_{B} N_{C} R N_{D} G T T G T A A C N_{D}
A (N_{A})₃-3′

or an analogue or derivative of said sequence.

In a third embodiment of the first aspect, said first strand comprises the sequence:

(SEQ ID NO: 58)

5′-(N_{A})₁₋₁₅ N_{E} N_{E} N_{B} N_{B} N_{C} R T G T T G T A A C T A
A A G-3′

or an analogue or derivative of said sequence.

In a fourth embodiment of the first aspect, said second strand comprises the sequence:

(SEQ ID NO: 59)

5′-(N_{A})₃ T A G T T A C A A C A T A C N_{B} N_{E} N_{E}
(N_{A})₁₋₁₅-3′

or an analogue or derivative of said sequence.

In a fifth embodiment of the first aspect, said second strand comprises the sequence:

(SEQ ID NO: 60)

5′-C T T T A G T T A C A A C A T A C N_{B} N_{E} N_{E}
(N_{A})₁₋₁₅-3′

or an analogue or derivative of said sequence.

According to a second aspect of the present invention, there is provided a conjugate, wherein said conjugate comprises a double-stranded oligonucleotide of the first aspect bound to one or more proteinaceous molecules, nucleic acid molecules, or small molecules.

The binding may be covalent or non-covalent.

The non-covalent binding of the double-stranded oligonucleotide may be to a Tus polypeptide.

The Tus polypeptide may have TerB-binding activity.

The Tus polypeptide may comprise the sequence set forth as SEQ ID NO: 5.

According to a third aspect of the present invention, there is provided a conjugate, wherein said conjugate comprises a double-stranded oligonucleotide of the first aspect bound to:

(i) a Tus polypeptide; and

(ii) a proteinaceous molecule, nucleic acid molecule, or small molecule.

The Tus polypeptide may have TerB-binding activity.

The Tus polypeptide may comprise the sequence set forth as SEQ ID NO: 5.

The double-stranded oligonucleotide may be derivatized to include 5′- and/or 3′-insertions that do not adversely affect its ability to bind to a Tus protein.

The insertions may include the addition of mRNA and/or DNA that is to be presented or displayed.

According to a fourth aspect of the present invention, there is provided use of a conjugate of the second or third aspects for presentation or display.

According to a fifth aspect of the present invention, there is provided a kit comprising a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce a double-stranded oligonucleotide of the first aspect.

According to a sixth aspect of the present invention, there is provided a kit for presenting or displaying a first molecule, wherein said first molecule comprises a double-stranded oligonucleotide of the first aspect, in a form suitable for conjugating to:

(a) a second molecule, wherein said second molecule comprises a nucleic acid, polypeptide or small molecule; and

(b) an integer selected from the group consisting of:
(i) a Tus polypeptide in a form suitable for conjugating to another molecule, wherein said double-stranded oligonucleotide and said Tus polypeptide interact in use to present or display another molecule conjugated to said double-stranded oligonucleotide or said polypeptide; and
(ii) mRNA encoding a Tus polypeptide in a form suitable for conjugating to mRNA encoding another polypeptide.

According to a seventh aspect of the present invention, there is provided a method for presenting or displaying a molecule on a surface, wherein said method comprises contacting a conjugate, wherein said conjugate comprises a double-stranded oligonucleotide of the first aspect covalently bound to the molecule, with a Tus polypeptide bound to the surface, for a time and under conditions sufficient to form a DNA/protein complex, wherein said molecule is displayed on the surface.

The molecule may comprise a polypeptide, nucleic acid, antibody or small molecule.

According to an eighth aspect of the present invention, there is provided a method for presenting or displaying a molecule on a surface, wherein said method comprises contacting a conjugate, wherein said conjugate comprises a Tus polypeptide covalently bound to the molecule, to a double-stranded oligonucleotide of the first aspect bound to the surface, for a time and under conditions sufficient to form a DNA/protein complex, wherein the molecule is displayed on the surface.

According to a ninth aspect of the present invention, there is provided a method for presenting or displaying a molecule, wherein said method comprises:

(i) incubating an mRNA conjugate, wherein said mRNA conjugate comprises mRNA encoding a Tus polypeptide fused to mRNA encoding a second polypeptide, for a time and under conditions sufficient for translation of the Tus polypeptide to be produced, and partial or complete translation of the mRNA encoding the second polypeptide to occur, thereby producing a complex comprising the conjugate, a nascent Tus-polypeptide fusion protein encoded by the conjugate and optionally a ribosome;

(ii) incubating the complex with a double-stranded oligonucleotide of the first aspect for a time and under conditions sufficient to bind to said Tus polypeptide; and

(iii) recovering the complex.

The mRNA encoding the Tus polypeptide may be fused to mRNA encoding a second polypeptide in the same reading frame.

According to a tenth aspect of the present invention, there is provided a method for the production of a conjugate comprising an oligonucleotide of the first aspect and a peptide, polypeptide or protein, wherein said method comprises:

(i) producing or synthesising said oligonucleotide bound to an agent capable of forming a bond with a peptide, polypeptide or protein; and

(ii) contacting the oligonucleotide with the peptide, polypeptide or protein for a time and under conditions sufficient for a bond to form between the agent and the peptide, polypeptide or protein.

According to an eleventh aspect of the present invention, there is provided a method for the production of a conjugate comprising an oligonucleotide of the first aspect and a Tus polypeptide, wherein said method comprises:

(i) producing or synthesising said oligonucleotide bound to an agent capable of forming a bond with a peptide, polypeptide or protein; and

(ii) contacting the oligonucleotide with the Tus polypeptide for a time and under conditions sufficient for a bond to form between the agent and the peptide, polypeptide or protein.

According to a twelfth aspect of the present invention, there is provided a process for presenting or displaying a molecule, wherein said process comprises:

(i) providing DNA encoding a fusion protein comprising a Tus polypeptide fused to a polypeptide of interest;

(ii) transcribing the DNA in the presence of an RNA polymerase to produce an mRNA conjugate comprising mRNA encoding a Tus polypeptide fused to mRNA encoding the polypeptide of interest;

(iii) incubating the mRNA conjugate for a time and under conditions sufficient for translation of a Tus polypeptide to be produced, and partial or complete translation of the mRNA encoding the polypeptide of interest to occur, thereby producing a complex comprising the conjugate, a nascent Tus-fusion protein encoded by the conjugate and optionally a ribosome;

(iv) incubating the complex with a double-stranded oligonucleotide of the first aspect for a time and under conditions sufficient to bind to said Tus polypeptide; and

(v) recovering the complex.

According to a thirteenth aspect of the present invention, there is provided a fusion protein comprising a Tus protein and a peptide, polypeptide or protein of interest for use in the method of the ninth aspect or the process of the eleventh aspect.

According to a fourteenth aspect of the present invention, there is provided a polynucleotide encoding the fusion protein of the thirteenth aspect.

According to a fifteenth aspect of the present invention, there is provided a vector containing the polynucleotide of the fourteenth aspect.

According to a sixteenth aspect of the present invention, there is provided a host cell transformed with the vector of the fifteenth aspect.

According to a seventeenth aspect of the present invention, there is provided a chip, wherein said chip comprises the double-stranded oligonucleotide of the first aspect or the conjugate of the second or third aspect.

### BRIEF DESCRIPTION OF THE DRAWINGS

FIG. 1: provides a schematic representation of the 5′-biotinylated forked (BF) TerB ligand BFTerB comprising a double-stranded oligonucleotide of the present invention conjugated to biotin and the formation of monomeric and quaternary complexes comprising BFTerB and streptavidin (SA).

FIG. 2: provides a schematic representation showing the preparation and application of a Tus surface. Tus is immobilized, and then BFTerB is immobilized and streptavidin (SA) is bound to the biotin moiety of BFTerB

FIG. 3: provides a graphical representation comparing the binding of BFTerB to either streptavidin (SA) or Tus-derivatized BIAcore chip surfaces. Injection of 250 nM BFTerB (1). Dissociation (2). Reinjection of 250 nM BFTerB (3). Dissociation (4). Injection of 2 mM SA (5). Injection stop (6).

FIG. 4: is a graphical representation showing the salt dependence of the dissociation of the complex of Tus with BFTerB. The BIAcore running buffer contains 150 mM salt. Between (1) and (2), the effects on dissociation with different NaCl concentrations ranging from 0-300 mM were monitored.

FIG. 5: is a graphical representation showing the stability of the Tus-BFTerB interaction overnight, using BFTerB bound to a Tus surface (BIAcore). Two streptavidin (SA) injections were used to report the amount of BFTerB still displayed on the surface.

FIG. 6: is a schematic representation showing the polarity of termination of E. coli chromosomal DNA replication at Tus-bound Ter sites. Panel (a) shows replication initiates at oriC and proceeds bi-directionally. The clockwise-moving replication fork passes through the clockwise-oriented Ter sites (i.e., TerH, I, E, D, A), but is arrested at Tus complexes at the counter-clockwise-oriented Ter sites (i.e., TerC, B, F, G, J). The opposite is true for the fork that moves in the counter clockwise direction. Panel (b) shows sequences of the first strands of ten naturally-occurring Ter sites designated TerB (SEQ ID NO: 6), TerA (SEQ ID NO: 7), TerC (SEQ ID NO: 8), TerD (SEQ ID NO: 9), TerE (SEQ ID NO: 10), TerF (SEQ ID NO: 11), TerG (SEQ ID NO: 12), TerH (SEQ ID NO: 13), TerI (SEQ ID NO: 14) and TerJ (SEQ ID NO: 15), from top to bottom of the Figure. When the Ter sites are bound to Tus, forks progressing from the non-permissive face are blocked, while those entering from the permissive face pass through. The 21-bp TerB sequence is highlighted. A conserved G-C base pair involved in fork arrest is indicated by the highlighted G in the first strand of the TerB sequence and each sequence below TerB.

FIG. 7: A. and B. provide schematic representations showing the thermodynamic and kinetic parameters for binding of Tus to modified TerB oligonucleotides as determined by BIAcore measurements in 250 mM KCl at 20° C. A conserved C residue in the second strand of naturally-occurring Ter sites that is involved in fork arrest (FIG. 6) is shown as an open circle on the second strand in each case. Data indicate the association rate constant as 10⁻⁶×kₐ (units M⁻¹s⁻¹) and the dissociation rate constant as 10³×k_{d} (units s⁻¹) (FIG. 7A) and half-life (s) (FIG. 7B). The nucleotide sequence of the first strand (SEQ ID NO: 16) and second strand (SEQ ID NO: 17) of a naturally-occurring TerB site are indicated at the top of the figure, wherein a conserved C residue present in naturally-occurring Ter sites and involved in fork arrest (FIG. 6) is enlarged. Non-forked control oligonucleotides comprised naturally-occurring TerB sequences having a 5′-biotinylated 10-residue abasic spacer (B) on the first strand (TerB) or second strand (rTerB). Forked structures were produced by substituting residues in the naturally-occurring TerB sequence for two or more other residues which are indicated by lighter shading. The nomenclature F2p, F3p, F4p indicates the substitution of 2, 3 or 4 nucleotides respectively, at the 3′-end of the first strand. The nomenclature F2n, F3n, F4n, F5n, F6n, F7n indicates the substitution of 2, 3, 4, 5, 6 or 7 nucleotides respectively, at the 5′-end of the first strand (e.g., as in F3n-rTerB, F4n-rTerB, F5n-rTerB, F6n-rTerB, F7n-rTerB) or at the 3′-end of the second strand (e.g., as in F3n-TerB, F4n-TerB, F5n-TerB). The nomenclature F5 indicates the substitution of 5 nucleotides at the 5′-end of the first strand (e.g., as in F5-TerB(G2), F5-TerB(G3), F5-TerB(G4), F5-TerB(G5), F5-TerB(A6). The nomenclature G2, G3, G4, G5, C6 indicates the substitution of a single naturally-occurring residue of TerB for G (e.g., as in F5-TerB(G2), F5-TerB(G3), F5-TerB(G4), F5-TerB(G5)) or C (e.g., as in F5-TerB(A6)) at position 1, 2, 3, 4 or 5 respectively, from the 3′-end of the second strand. The nomenclature A5n indicates the deletion of 5 nucleotides from the 5′-end of the first strand relative to naturally-occurring TerB (e.g., as in A5n-rTerB). The nomenclature TerB indicates that a 5′-biotinylated 10-residue abasic spacer (B) has been added to the first strand. The nomenclature rTerB indicates that a 5′-biotinylated 10-residue abasic spacer (B) has been added to the second strand. TerB variants that have the ability to bind Tus at a higher affinity (i.e., reduced Kₐ value) than naturally-occurring TerB are indicated as “non-permissive” variants. The half life for dissociation of TerB from Tus as determined by BIAcore measurements at 20° C. is about 140 seconds (k_{d} of about 0.005 s⁻¹). Those TerB variants having higher half lives for dissociation of Tus (i.e., reduced k_{d} value) as determined by BIAcore measurements at 20° C. include F5n-rTerB, half life of about 5300 seconds; A5n-rTerB, half life of about 6900 seconds; F6n-rTerB, half life of about 6900 seconds; F7n-rTerB, half life of about 2900 seconds; F5-TerB(G2), half life of about 4300 seconds; F5-TerB(G3), half life of about 5000 seconds; F5-TerB(G4), half life of about 5000 seconds; and F5-TerB(G5), half life of about 2300 seconds.

FIG. 8: A.-C. provide schematic representations showing the thermodynamic and kinetic parameters for binding of Tus to modified TerB oligonucleotides as determined by BIAcore measurements in 250 mM KCl at 20° C. Data indicate the association rate constant as 10⁻⁶×kₐ (units M⁻¹s⁻¹), the dissociation rate constant as 10³×k_{d} (units s⁻¹), the ratio of k_{d}/kₐ (nM), dissociation equilibrium constant K_{D} (nM) and half-life for dissociation of Tus (min). Sequences of oligonucleotides are indicated in doubled-stranded format and the corresponding SEQ ID NOs for first (top) and second (lower) strands indicated below in ascending numerical order for each pair. The nomenclature of oligonucleotides is as described in the legend to FIG. 7 except that the biotin tag is indicated by “5′-Bio - - -” or “- - - Bio-5′ ” depending upon its orientation. The oligonucleotides shown in FIG. 8 that are designated TerB (SEQ ID NOs: 16 and 17), rTerB (SEQ ID NOs: 18 and 19), F2p-rTerB (SEQ ID NOs: 19 and 20), F3p-rTerB (SEQ ID NOs: 19 and 21), F3p-TerB (SEQ ID NOs: 16 and 22), F4p-rTerB (SEQ ID NOs: 19 and 23), F4p-TerB (SEQ ID NOs: 16 and 24), F3n-TerB (SEQ ID NOs: 16 and 25), F3n-rTerB (SEQ ID NOs: 19 and 26), F4n-TerB (SEQ ID NOs: 16 and 27), F4n-rTerB (SEQ ID NOs: 19 and 28), F5n-TerB (SEQ ID NOs: 16 and 29), FSn-rTerB (SEQ ID NOs: 19 and 30), A5n-rTerB (SEQ ID NOs: 19 and 31), F6n-rTerB (SEQ ID NOs: 19 and 32), F7n-rTerB (SEQ ID NOs: 19 and 33), F5-TerB(G2) (SEQ ID NOs: 34 and 35), F5-TerB(G3) (SEQ ID NOs: 34 and 36), F5-TerB(G4) (SEQ ID NOs: 34 and 37), F5-TerB(G5) (SEQ ID NOs: 34 and 38) and F5-TerB(C6) (SEQ ID NOs: 34 and 39) are also represented schematically in FIG. 7. For the oligonucleotides in FIG. 8 designated Δ4p-rTerB (SEQ ID NOs: 19 and 40), Δ4p-TerB (SEQ ID NOs: 16 and 41), A3n-TerB (SEQ ID NOs: 16 and 42) and A3n-rTerB (SEQ ID NOs: 19 and 43), the term Δ4p indicates the deletion of 4 nucleotides from the 3′-end of the first strand relative to naturally-occurring TerB (e.g., as in Δ4p-rTerB) or from the 5′-end of the second strand relative to naturally-occurring TerB (e.g., as in Δ4p-TerB), and the term A3n indicates the deletion of 3 nucleotides from the 5′-end of the first strand relative to naturally-occurring TerB (e.g., as in A3n-rTerB) or from the 3′-end of the second strand relative to naturally-occurring TerB (e.g., as in A3n-TerB). The double-stranded oligonucleotide designated F5-TerB(G2) was further mutated by deletion of the four 3′-terminal nucleotides from the second strand to produce the oligonucleotide designated “single O/H C” (SEQ ID NOs: 34 and 44) in FIG. 8. The deoxyribonucleotide analogues 5′-bromo deoxyuridine (5′BrdU; indicated by # in the Figure) or 5′-iodo deoxyuridine (5′IdU; indicated by # in the Figure) were also incorporated into the second strand of a naturally-occurring TerB sequence (SEQ ID NO: 17) and annealed to the first strand biotinylated TerB sequence (SEQ ID NO: 16) to produce the double-stranded oligonucleotides designated Bromo-TerB (SEQ ID NOs: 16 and 45) and Iodo-TerB (SEQ ID NOs: 16 and 46), respectively in FIG. 8b. The deoxyribonucleotide analogues 5′BrdU (indicated by # in the Figure) or 5′IdU (indicated by # in the Figure) were also incorporated into the second strand of a naturally-occurring TerB sequence (SEQ ID NO: 17) and annealed to the first strand biotinylated sequence of F5n-TerB(G2) (SEQ ID NO: 34) to produce the double-stranded oligonucleotides designated Bromo-Lock (SEQ ID NOs: 34 and 45) and Iodo-Lock (SEQ ID NOs: 34 and 46), respectively in FIG. 8. As indicated in FIG. 8, longer oligonucleotides were also produced, for example a double-stranded oligonucleotide comprising an additional 14 nucleotides at the 5′-end of the first strand of a naturally-occurring TerB sequence and the corresponding additional complementary sequence at the 3′-end of the second strand (e.g., Ext-rTerB, SEQ ID NOs: 47 and 48) with 1, 2, 3, 4, or 5 nucleotide substitutions were introduced to the first strand at a location within the TerB core (e.g., 1 mismatch, SEQ ID NOs: 48 and 49; 2 mismatch, SEQ ID NOs: 48 and 50; 3 mismatch, SEQ ID NOs: 48 and 51; 4 mismatch, SEQ ID NOs: 48 and 52; 5 mismatch, SEQ ID NOs: 48 and 53). Finally, a single nucleotide deletion was produced within the first strand of Ext-rTerB to disrupt base-pairing of (i.e., “flip-out”) the C residue present in naturally-occurring Ter sites (FIG. 6) e.g., “bulged C6” (SEQ ID NOs: 48 and 54). Kinetic data indicate that the oligonucleotides designated F5n-rTerB, A5n-rTerB, F6n-rTerB, F7n-rTerB, F5-TerB(G2), F5-TerB(G3), F5-TerB(G4), F5-TerB(G5), single O/H C, Bromo-TerB, Bromo-Lock, Iodo-terB, Iodo-Lock, Ext-rTerB, 1 mismatch, 2 mismatch, 3 mismatch, 4 mismatch, 5 mismatch are suitable for binding to Tus.

FIG. 9: is a graphical representation of BIAcore sensor grams showing binding of Tus to TerB oligonucleotides modified at the permissive face (i.e., in 250 mM KCl, at 20° C., 4 min injection of 20 nM Tus). Oligonucleotides are named as in FIGS. 7 and 8. Data show that, as the forks increase in length with mutations on either strand, dissociation rates become progressively faster.

FIG. 10: is a graphical representation of BIAcore sensor grams showing binding of Tus to TerB oligonucleotides modified at the non-permissive face (i.e., in 250 mM KCl, at 20° C., 4 min injection of 20 nM Tus). Oligonucleotides are named as in FIGS. 7 and 8. Forks with up to four mismatches on the 5′ strand (e.g., F4n-rTerB) or up to five mismatches on the 3′ strand (e.g., F5n-TerB) show kinetic behaviour similar to that of the wild-type TerB oligonucleotide.

FIG. 11: is a graphical representation of BIAcore sensor grams showing binding of Tus to TerB oligonucleotides modified at the non-permissive face (i.e., in 250 mM KCl, at 20° C., 2 min injection of 100 nM Tus). Oligonucleotides are named as in FIGS. 7 and 8. Forks for which the conserved C residue in the second strand of Ter sites (FIG. 6) is mispaired are marked with an asterisk and shown to exhibit very slow dissociation rates (i.e. a “locked” behaviour).

FIG. 12: is a graphical representation of BIAcore sensor grams showing binding of Tus to TerB oligonucleotides modified at the non-permissive face (i.e., in 250 mM KCl, at 20° C., 2 min injection of 20 mM Tus). Oligonucleotides are named as in FIGS. 7 and 8. Data show that those forks for which the conserved C residue in the second strand of Ter sites (FIG. 6) is present and mispaired (marked with an asterisk) exhibit very slow dissociation rates (“locked” behaviour).

FIG. 13: is a schematic representation showing examples of transcription units inserted downstream of the T7 promoter (T7p) in pET plasmids: His6, region encoding hexaHis tag; gene, an open reading frame (ORF) of interest or library of ORFs (e.g., encoding Tus/9Ala-Tus polypeptide, CyPA/PpiB); PSA, sequence encoding poly(Ser-Ala)₁₅ C-terminal tail; RBS, ribosome-binding site; RR, sequence encoding random RNA sequence; TerB, a Tus-binding site; Lin, sequence encoding a flexible linker; Nd, RI, N_{C}, H: restriction sites used for library construction and linearization of construct for runoff transcription. End-filling and religation of the EcoRI site (RI) results in creation of an in-frame TAA stop codon (+/− STOP).

FIG. 14: is a schematic representation showing methods for attachment of TerB ds-DNA at the 3′ end of mRNA.

FIG. 15: depicts a model representation of the exonuclease assay. The SA, Bio-Tus, (dT)₅₀[TT-lock](dT)₅₀ substrate and ε186 are respectively depicted by ovals, rectangles, ladders and crescents. A: Stable baseline after binding of Bio-Tus to the SA surface. B: Injection of the (dT)₅₀[TT-Lock](dT)₅₀ substrate, yielding a stable baseline. C: Injection of ε186 and start of exonuclease activity. The initial increase in response represents a binding event, and the following decrease represents loss of substrate through exonuclease action. D: End of injection of 6186, wherein all of the single-stranded region of the DNA substrate has been digested.

FIG. 16: shows concentration dependence of the exonuclease activity of 6186 during application of the TT-Lock to a regenerable surface plasmon resonance chip to monitor direct real-time kinetics of nucleases. Concentrations of 6186 are shown in B. All the plots were normalized to 200 RU of (dT)₅₀[TT-Lock](dT)₅₀ substrate binding.

FIG. 17: shows extension of forks at the permissive end of TerB resulting in progressively more rapid dissociation of Tus. A. Interaction of Tus with TerB oligonucleotides with forks at the permissive end. Half-lives and dissociation constants (K_{D}) of Tus-TerB complexes, as measured by SPR at 20° C. in buffer containing 0.25 M KCl. Base substitutions that replace the natural TerB sequence are shown together with the C(6) residue. “B-” denotes the strand that was modified with a 5′-biotinylated ten-residue abasic spacer. B. Representative Biacore sensorgrams with different oligonucleotides are shown for binding of 20 nM His₆-Tus. Data were normalized on the basis of the measured maximum response at saturating [Tus] (˜50 response units). C. Model for dissociation of Tus following DnaB-mediated strand separation at the permissive face of the Tus-Ter complex.

FIG. 18: depicts a molecular mousetrap determining polarity of replication fork arrest. A. Dissociation of Tus from complexes with TerB oligonucleotides forked at the non-permissive end. Half-lives and dissociation constants (K_{D}) of Tus-TerB complexes, as measured by SPR. Data for the TerB variants that show the “locked” behavior are shown. Base substitutions relative to the natural TerB sequence are also shown as is the C(6) residue. “B-” denotes the strand that was modified with a 5′-biotinylated ten-residue abasic spacer. B. The “locked” complex forms when the fork extends far enough to expose C(6) (in F5n-rTerB). Representative Biacore sensorgrams showing His₆-Tus (10 nM) binding to and dissociation from wild-type and forked TerB sequences. C. Strand specificity of “locking” behavior at the non-permissive end of TerB (Biacore sensorgrams; 10 nM Tus). D. A single nucleotide, C(6) of TerB is responsible for formation of the “locked” species: effect of base substitution on dissociation of Tus from forked TerB sequences. His₆-Tus was bound at a saturating concentration (100 nM) to forked TerB species containing mutations in T(2) to C(6). Tus formed a “lock” on all species except that in which C(6) was mutated to adenine (or guanosine or thymine; see panel A), indicating that C(6) is the critical base for “lock” formation. E. Mousetrap model for fork arrest at the non-permissive face. The trap is set by helicase action, and sprung by base-flipping of C(6) into a new binding site on the surface of Tus, resulting in a “locked” complex between Tus and forked Ter DNA.

FIG. 19: shows salt dependence of dissociation rate constants (k_{d}), at 20° C. The slopes of the least-squares fitted lines (log/log scales) were 6.8±0.4 and 0.60±0.08 for rTerB at low and high [KCl], respectively. Corresponding values for F5n-rTerB were 3.4±0.3 and 0.32±0.19.

FIG. 20: the “locked” complex has many interactions in common with the complex of Tus with double-stranded TerB, and is not formed simply by base-flipping of C(6). Half-lives and dissociation constants (K_{D}) of Tus-TerB complexes, as measured by SPR. A. Substitution of T(8) and T(19) of TerB with 5-bromo- (residues in green) or 5-iodo-dUMP (blue) stabilize Tus complexes with both duplex TerB and the “lock”, to similar extents. B. An extensive single-stranded “bubble”, as in oligonucleotide “5-mismatch” is required to form the “lock” structure, suggesting that “lock” formation does not simply require flipping of the C(6) base.

FIG. 21: depicts the structure of the “Tus-Ter lock”. A. Portion of the final 2Fₒ-F_{c} electron density map, contoured at la, showing the region of the displaced strand in the “Tus-Ter lock” complex. Comparison of structures of complexes of Tus with B. wild-type TerA (PDB code 1ECR) and with C. an oligonucleotide with a forked structure at the non-permissive face. D. Structure of the DNA-binding site at the non-permissive face in the wild-type complex, showing the movement of C(6) required to form the “locked” structure, as shown in E. F. Sequences of the oligonucleotides used for crystallization, with C(6) highlighted. Nucleotides shown in boxes represent those that were not visible in the structures of the complexes.

FIG. 22: shows “unlocking” of the “Tus-Ter lock” on approach of a second replisome to the permissive face, with dissociation of Tus from complexes with TerB oligonucleotides forked at both the permissive and non-permissive ends. Half-lives and dissociation constants (K_{D}) of Tus-TerB complexes, as measured by SPR, are shown.

### DEFINITIONS

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

The term “nucleic acid molecule” as used herein refers to a single- or double-stranded polymer of deoxyribonucleotide, ribonucleotide bases or known analogues of natural nucleotides, or mixtures thereof. The term includes reference to the specified sequence as well as to the sequence complementary thereto, unless otherwise indicated. The terms “nucleic acid” and “polynucleotide” are used herein interchangeably. It will be understood that “5′end” as used herein in relation to a nucleic acid molecule corresponds to the N-terminus of the encoded polypeptide and “3′end” corresponds to the C-terminus of the encoded polypeptide.

The terms “nucleic acid molecule”, “polynucleotide” and “oligonucleotide” are used interchangeably herein.

In the present context, the term “anneal” or “annealed” or similar term shall be taken to mean that the first and second strands are base-paired to each other to form a double-stranded nucleic acid, either spontaneously under the conditions in which the double-stranded oligonucleotide is employed or other conditions known in the art to promote or permit base-pairing between complementary nucleotide residues or induced to form such base-pairing. As will be known to the skilled artisan, two complementary single polynucleotides comprising RNA and/or DNA including one or more ribonucleotide analogues and/or deoxyribonucleotide analogues will generally anneal to form a double helix or duplex. As will be known to the skilled artisan, the ability to form a duplex and/or the stability of a formed duplex depend on one or more factors including the length of a region of complementarity between the first and second strands, the percentage content of adenine and thymine in a region of complementarity between the first and second strands (i.e., “A+T content”), the incubation temperature relative to the melting temperature (Tm) of a duplex, and the salt concentration of a buffer or other solution in which the first and second strands are incubated. Generally, to promote duplex formation, the nucleic acid strands are incubated at a temperature that is at least about 1-5° C. below a Tm of a duplex that is predicted from its A+T content and length. Duplex formation can also be enhanced or stabilized by increasing the amount of a salt (e.g., NaCl, MgCl₂, KCl, sodium citrate, etc), or by increasing the time period of the incubation, as described by Sambrook et al., Molecular Cloning: A Laboratory Manual, Cold Spring Harbor Laboratory Press; Hames and Higgins, Nucleic Acid Hybridization: A Practical Approach, IRL Press, Oxford (1985); Berger and Kimmel, Guide to Molecular Cloning Techniques, In: Methods in Enzymology, Vol 152, Academic Press, San Diego Calif. (1987); or Ausubel et al., Current Protocols in Molecular Biology, Wiley Interscience, ISBN 047150338 (1992).

The term “deoxyribonucleotide” is an art-recognized term referring to those bases of DNA each comprising phosphate, deoxyribose and a purine or pyrimidine base selected from the group consisting of adenine (A), cytidine (C), guanine (G) and thymine (T). In the triphosphate form, deoxyribonucleotide triphosphates (dNTPs), e.g., DATP, dCTP, cGTP and TTP, are capable of being incorporated into DNA by an enzyme of DNA synthesis e.g., a DNA polymerase.

The term “ribonucleotide” is an art-recognized term referring to those bases of RNA each comprising a purine or pyrimidine base selected from the group consisting of adenine (A), cytidine (C), guanine (G) and uracil (U) linked to ribose. Ribonucleotides are capable of being incorporated into RNA by an enzyme of RNA synthesis e.g., an RNA polymerase.

As used herein in respect of nucleic acids or oligonucleotides, the term is “upstream” shall be taken to mean that a stated integer e.g., a ribonucleotide, deoxyribonucleotide or analogue thereof, is positioned 5′ relative to a nucleotide sequence, albeit not necessarily at the 5′-terminus of said sequence or at the 5′-end of the nucleic acid containing the ribonucleotide, deoxyribonucleotide or analogue. Accordingly, a ribonucleotide, deoxyribonucleotide or analogue thereof positioned “upstream” of a nucleotide sequence may be internal by virtue of there being other residues positioned upstream of it. Alternatively, a ribonucleotide, deoxyribonucleotide or analogue thereof positioned “upstream” of a nucleotide sequence may be at the 5′-end.

Similarly, the term “downstream” shall be taken to mean that a stated integer e.g., a ribonucleotide, deoxyribonucleotide or analogue thereof, is positioned 3′ relative to a nucleotide sequence, albeit not necessarily at the 3′-terminus of said sequence or at the 3′-end of the nucleic acid containing the ribonucleotide, deoxyribonucleotide or analogue. Accordingly, a ribonucleotide, deoxyribonucleotide or analogue thereof positioned “downstream” of a nucleotide sequence may be internal by virtue of there being other residues positioned downstream of it. Alternatively, a ribonucleotide, deoxyribonucleotide or analogue thereof positioned “downstream” of a nucleotide sequence may be at the 3′-end.

The term “5′-terminus” or “5′-end” shall be taken to mean that a stated integer e.g., a ribonucleotide, deoxyribonucleotide or analogue thereof, is positioned 5′ relative to a nucleotide sequence such that it is at an end of nucleic acid containing the ribonucleotide, deoxyribonucleotide or analogue (i.e., there are no residues upstream of the stated integer).

The term “3′-terminus” or “3′-end” shall be taken to mean that a stated integer e.g., a ribonucleotide, deoxyribonucleotide or analogue thereof, is positioned 3′ relative to a nucleotide sequence such that it is at an end of nucleic acid containing the ribonucleotide, deoxyribonucleotide or analogue (i.e., there are no residues downstream of the stated integer).

The term “analogue” when used in relation to an oligonucleotide or residue thereof, means a compound having a physical structure that is related to a DNA or RNA molecule or residue, and preferably is capable of forming a hydrogen bond with a DNA or RNA residue or an analogue thereof (i.e., it is able to anneal with a DNA or RNA residue or an analogue thereof to form a base-pair). Such analogues may possess different chemical and biological properties to the ribonucleotide or deoxyribonucleotide residue to which they are structurally related. “Analogues” of the oligonucleotides of the present invention therefore include, for example, any functionally-equivalent nucleic acids that bind to a Tus protein and which include one or more analogues of A, C, G or T. For example, an analogue comprised of the nucleotide sequence of the first aspect may have one or more of the nucleotides A, C, G or T therein substituted for one or more nucleotide analogues. Methylated, iodinated, brominated or biotinylated residues are particularly preferred analogues. However, other analogues such as, for example, those analogues specified elsewhere herein, may also be used.

The term “derivative” when used in relation to the oligonucleotides of the present invention include any functionally-equivalent nucleic acids that bind to a Tus protein and which include one or more nucleotides and/or nucleotide analogues upstream or downstream, including any fusion molecules produced integrally (e.g., by recombinant means) or added post-synthesis (e.g., by chemical means). Such fusions may comprise one or both strands of the double-stranded oligonucleotide of the invention with RNA or DNA added thereto or conjugated to a polypeptide (e.g., puromycin or other polypeptide), a small molecule (e.g., psoralen) or an antibody. Particularly preferred derivatives include mRNA or DNA conjugated to the oligonucleotide of the invention for displaying on a microwell or microarray surface or on the surface of a cell, phage, virus or in vitro.

As used herein the term “polypeptide” means a polymer made up of amino acids linked together by peptide bonds. The term “polypeptide” may be used interchangeably with the term “protein” and includes fragments, variants and analogues thereof.

The term “fragment” when used in relation to a polypeptide or polynucleotide molecule refers to a constituent of a polypeptide or polynucleotide. Typically the fragment possesses qualitative biological activity in common with the polypeptide or polynucleotide. However, fragments of a polynucleotide do not necessarily need to encode polypeptides which retain biological activity. Rather, a fragment may, for example, be useful as a hybridization probe or PCR primer. The fragment may be derived from a polynucleotide of the invention or alternatively may be synthesized by some other means, for example chemical synthesis.

The term “variant” as used herein refers to substantially similar sequences. Generally, polypeptide or polynucleotide sequence variants possess qualitative biological activity in common. Further, these polypeptide or polynucleotide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of polypeptides or polynucleotides of the invention. A homologue is typically a polypeptide or polynucleotide from a different species but sharing substantially the same biological function or activity as the corresponding polypeptide or polynucleotide disclosed herein.

The term “analogue” as used herein with reference to a polypeptide means a polypeptide which is a derivative of the polypeptide of the invention, which derivative comprises addition, deletion, substitution of one or more amino acids, such that the polypeptide retains substantially the same function.

The term “purified” means that the material in question has been removed from its natural environment or host, and associated impurities reduced or eliminated such that the molecule in question is the predominant species present. Thus, essentially, the term “purified” means that an object species is the predominant species present (ie., on a molar basis it is more abundant than any other individual species in the composition), and preferably a substantially purified fraction is a composition wherein the object species comprises at least about 30 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. The terms “purified” and “isolated” may be used interchangeably.

As used herein, the term “Tus protein” refers to any polypeptide capable of binding to a Ter site, including a full-length naturally-occurring Tus polypeptide or a fragment or other derivative thereof having Ter binding activity or a variant, homologue or analogue thereof having Ter-binding activity.

For example, the term “Tus” includes any peptide, polypeptide, or protein having at least about 80% amino acid sequence identity to the amino acid sequence of E. coli Tus polypeptide set forth in SEQ ID NO: 5 wherein said polypeptide has Ter binding activity.

As used herein, the term “proteinaceous” shall be taken to include a cell, virus particle, bacteriophage, ribosome, polypeptide or a polypeptide fragment or a synthetic peptide.

As used herein, the term “conjugate” shall be taken to mean a composition of matter wherein one integer is covalently attached or produced integrally with a second integer. For example, a strand of the oligonucleotide of the present invention may be synthesized as a DNA/RNA hybrid molecule to integrate an mRNA molecule. Similarly, the strands of the double-stranded oligonucleotide may be synthesized to comprise additional sequence of a double-stranded oligonucleotide. Alternatively, a nucleic acid (DNA or RNA), polypeptide (e.g., a puromycin conjugate) or small molecule (e.g., a psoralen or derivative thereof) may be added post-synthetically to the double-stranded oligonucleotide by any conventional means known in the art.

As used herein, the term “chip” includes an array or microarray of any description, and includes a surface plasmon resonance chip, or “Biacore” chip. In particular, the term “chip” includes the chips referred to Example 4 disclosed herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### DETAILED DESCRIPTION OF THE PREFERRED EMBODIMENTS

In work leading up to the present invention, the inventors sought to produce a cost-effective, reusable means for presenting targets (e.g., nucleic acid, protein, polypeptide, peptide, antibody or fragment thereof, or small molecule) for the purposes of molecular screening. The inventors sought to produce such means for use in a variety of applications, including for example screening platforms for nucleic acids, proteins, antibodies, and small molecules, and in particular for pharmaceutical and agrochemical screening platforms.

Based upon an understanding of the interaction between the Escherichia coli termination site (TerB) and the E. coli replication terminator protein Tus, the inventors have developed a novel double-stranded and forked nucleic acid designated “TT-Lock” that is suitable for the above-mentioned applications. The inventors have identified a minimum nucleotide sequence of the double-stranded TT-Lock oligonucleotide that is required for high affinity binding to Tus, with a slow dissociation constant of the resultant TT-Lock/Tus complexes.

The minimum nucleotide sequence of the TT-Lock oligonucleotide may be about 13 nucleotides in length and may comprise a 3′-cytosine overhang on one strand which may lock the double-stranded oligonucleotide into a complex with Tus that is at least about 10 times more stable than the naturally-occurring complex between TerB and Tus protein. This 3′-cytosine overhang may also reduce the rate of dissociation of the complex between the TT-Lock and Tus protein compared to the naturally-occurring is complex between TerB and Tus protein.

The increased or enhanced stability of the interaction between TT-Lock and Tus protein and the reduced dissociation of TT-Lock/Tus complexes compared to the naturally-occurring counterpart renders the TT-Lock suitable for commercial applications. As exemplified herein, the inventors have immobilized Tus protein onto a surface plasmon resonance chip and shown that the double-stranded TT-Lock oligonucleotide of the present invention conjugated via a biotin moiety to a streptavidin protein is captured by, or binds to, the immobilized Tus protein at an affinity similar to that of the interaction between streptavidin and biotin. The Tus-coated chips were found to be capable of capturing or binding TT-Lock at high affinity following stripping of the TT-Lock/streptavidin conjugate. Additionally, the inventors have found that the Tus-coated chips with TT-Lock/streptavidin conjugate bound thereto are stable for extended periods of time, thereby conferring an ability to store such chips in a ready-to-use form prior to use.

### Oligonucleotide Synthesis

The oligonucleotides of the present invention may be produced by recombinant or chemical means known to the skilled artisan. As the oligonucleotides of the present invention may be less than about 100 nucleotides in length, and in particular may be no more than about 30 or 35 or 40 or 45 or 50 nucleotides in length, and may not comprise completely complementary first and second strands, chemical synthesis of each strand separately, followed by annealing of the first and second strands under appropriate hybridization conditions may be preferred.

DNA of up to about 80 nucleotides in length may be conveniently synthesized by chemical means. Longer molecules may generally be manufactured by amplification using PCR directly from template DNA by annealing overlapping oligonucleotide primers and primer extension of the overlapping ends to produce a full-length double-stranded nucleic acid molecule, for example, as described by Stemmer et al., Gene 164, 49-53, 1995; Casimiro et al., Structure 5, 1407-1412, 1997.

The solid phase chemical synthesis of DNA fragments may be routinely performed using protected nucleoside phosphoramidites, for example, as described by Beaucage et al., Tetrahedron Lett. 22, 1859, 1981. In general, the 3′-hydroxyl group of an initial 5′-protected nucleoside may be covalently attached to a polymer resin support, for example, as described by Pless et al., Nucleic Acids Res. 2, 773, 1975. Synthesis of the oligonucleotide may then proceed by deprotection of the 5′-hydroxyl group of the attached nucleoside, followed by coupling of an incoming nucleoside-3′-phosphoramidite to the deprotected hydroxyl group, for example, as described by Matteucci et al., J. Am. Chem. Soc. 103, 3185, 1981. The resulting phosphite triester may be oxidized to a phosphorotriester to complete the internucleotide bond (see, for example, Letsinger et al., J. Am. Chem. Soc. 98, 3655, 1976. The steps of deprotection, coupling and oxidation may be repeated until an oligonucleotide of the desired length and sequence is obtained.

The chemical group conventionally used for the protection of nucleoside 5′-hydroxyls may be dimethoxytrityl (“DMT”), which is removable using acid (Khorana, Pure Appl. Chem. 17, 349, 1968; Smith et al, J. Am. Chem. Soc. 84, 430, 1962) and may aid separation on reverse-phase HPLC (Becker et al., J. Chromatogr. 326, 219 (1985)). Alternatively, 5′-O-protecting groups which may be removed under non-acidic conditions may be used, for example, as described by Letsinger et al., J. Am. Chem. Soc. 89, 7147, 1967; Iwai et al., Tetrahedron Lett. 29, 5383, 1988; Iwai et al., Nucleic Acids Res. 16, 9443, 1988. Seliger et al., Nucleosides & Nucleotides 4, 153, 1985 also describe a 5′-O-phenyl-azophenyl carbonyl (“PAPco”) group, which may be removed by a two-step procedure involving trans-esterification followed by beta-elimination. Fukuda et al., Nucleic Acids Res. Symposium Ser. 19, 13, 1988, and Lehmann et al., Nucleic Acids Res. 17, 2389, 1989 also describe application of a 9-fluorenylmethylcarbonate (“Fmoc”) group for 5′-protection which produces yields for the synthesis of oligonucleotides up to 20 nucleotides in length. Letsinger et al., J. Am. Chem. Soc. 32, 296, 1967 also describe the use of a p-nitrophenyloxycarbonyl group for 5′-hydroxyl protection. Dellinger et al., US Patent Publication No. 20040230052 (18 Nov. 2004) also describe rapid and selective deprotection of 5′-OH or 3′-OH nucleoside carbonate groups using peroxy anions in aqueous solution, at neutral or mild pH.

Means for chemically synthesizing RNA are described, for example, in US Patent Publication No. 0040242530 (2 Dec. 2004) which is incorporated herein in its entirety. These methods rely upon 5′-DMT-2′-t-butyldimethylsilyl (TBDMS) or 5′-DMT-2′-[1-(2-fluorophenyl)-4-methoxypiperidin-4-yl] (FPMP) chemistries that are readily available commercially.

In summary, nucleosides may be suitably protected and functionalized for use in solid-phase or solution-phase synthesis of RNA oligonucleotides. For example, syntheses may be performed on derivatized polymer supports using either a Gene Assembler Plus synthesizer (Pharmacia) or a 380B synthesizer (ABI). A 2′-hydroxyl group in a ribonucleotide may be modified using a Tris orthoester reagent, to yield a 2′-O-orthoester nucleoside, by reacting the ribonucleoside with the tris orthoester reagent in the presence of an acidic catalyst, for example, pyridinium p-toluene sulfonate. The product may then be subjected to protecting group reactions (e.g., 5′-O-silylation) and functionalizations (e.g., 3′-O-phosphitylation) to produce a nucleoside phosphoramidite for incorporation within an oligonucleotide or polymer by reactions known to those skilled in the art. Following synthesis, the polymer support may be treated to cleave the protecting groups from the phosphates (including base-labile protecting groups) and to release the 2′-protected RNA oligonucleotide into solution. Crude reaction mixtures may then be analyzed by anion exchange high pressure liquid chromatography (HPLC) and subjected to sequence analysis.

RNA may also be produced by in vitro transcription of DNA encoding each strand of a double-stranded oligonucleotide of the invention, for example, by being cloned into a plasmid vector or an oligonucleotide template using an RNA polymerase enzyme, for example, E. coli RNA polymerase, bacteriophage SP6, T3, T7 RNA polymerase, an error-prone RNA polymerase such as Qβ-replicase or other viral polymerase. In vitro methods for synthesizing single stranded RNAs of defined length and sequence using RNA polymerase are described by Milligan et al., Nucleic Acid Res. 15, 8783-8798, 1987 and in US Patent Publication No. 20040259097 (23 Dec. 2004).

For the production of double-stranded RNA using an RNA polymerase, both a sense and an antisense oligonucleotide template may be required to be separately transcribed and the reaction products annealed. The oligonucleotide templates may be synthetic DNA templates or templates generated as linearized plasmid DNA from a target-specific sequence cloned into a restriction site of a vector such as for example a prokaryotic cloning vector (pUC13, pUC19) or PCR cloning systems such as the TOPO cloning system of Invitrogen. Synthetic DNA templates may be produced according to techniques well known in the art.

An RNA polymerase enzyme may form an RNA polymer from ribonucleoside 5′-triphosphates that is complementary to the DNA template. The enzyme may add mononucleotide units to the 3′-hydroxyl ends of the RNA chain and thus build RNA in the 5′-to-3′ direction, antiparallel to the DNA strand used as template. DNA-dependent RNA polymerases such as E. coli RNA polymerase, RNA-directed RNA polymerases such as the bacteriophage RNA polymerases (i.e., RNA replicases), or bacterial polynucleotide phosphorylases may be used in this context.

RNA polymerases generally require the presence of a specific initiation site or RNA polymerase promoter sequence within each DNA template to bind the RNA polymerase and initiate transcription. A minimum or truncated RNA polymerase promoter sequence, wherein one or more nucleotides of a naturally-occurring promoter sequence are deleted may also be employed, with no or little effect on the binding of the RNA polymerase to the initiation site and with no or little effect on the transcription reaction.

The reaction conditions for transcription reactions performed in vitro are known in the art to comprise a DNA template, an RNA polymerase enzyme and the nucleoside triphosphates (NTPs) for the four required ribonucleotide bases, adenine, cytosine, guanine and uracil, in a reaction buffer optimal for the RNA polymerase enzyme activity. For example, the reaction mixture for an in vitro transcription using T7 RNA polymerase typically contains, T7 RNA polymerase (0.05 mg/nl), oligonucleotide templates (1 μM), each NTP (4 mM), and MgCl₂ (25 mM) which supplies Mg²⁺ as a co-factor for the polymerase. This mixture may be incubated at about 37° C. in a buffer comprising 10 mM Tris-HCl pH 8.1 for several hours (see Milligan & Uhlenbeck, Methods Enzymol 180, 51-62, 1989). Such reagents are commercially available e.g., MEGA shortscript T7 kit (Ambion).

The oligoribonucleotide transcription products may be purified by any method known in the art such as, for example, gel electrophoresis, size exclusion chromatography, capillary electrophoresis or HPLC. Gel electrophoresis may be typically used to purify the full-length transcripts from the reaction mixture, but this technique may not be amenable to production on a large scale. Size exclusion chromatography, such as using Sephadex G-25 resin (Pharmacia), optionally combined with a phenol:chloroform:isoamyl alcohol extraction and ethanol precipitation may be more appropriate for large scale preparations.

To obtain double-stranded DNA (dsDNA) or double-stranded RNA (dsRNA) or a double-stranded hybrid molecule such as an RNA/DNA hybrid, the two strands may be annealed by standard means known to the skilled artisan. For example, the first and second strands may be brought into contact with each other at a temperature below their predicted Tm and/or in a medium comprising a salt such as KCl, MgCl₂ or NaCl.

### TT-Lock Oligonucleotides

In one embodiment, the present invention provides a double-stranded oligonucleotide, wherein said oligonucleotide comprises a first strand and a second strand, wherein:

(a) said first strand comprises the sequence:

(SEQ ID NO: 1)

5′-N_{C} R N_{D} G T T G T A A C N_{D} A-3′

or an analogue or derivative of said sequence; and

(b) said second strand comprises the sequence:

(SEQ ID NO: 2)

5′-T N_{D} G T T A C A A C N_{D} T N_{C} C-3′

or an analogue or derivative of said sequence

wherein R is a purine, N_{C} and N_{D} are each a DNA or RNA residue or analogue thereof, N_{C} residues in said first strand and said second strand may or may not be complementary, and N_{D} residues in said first strand and said second strand are sufficiently complementary to permit said N_{D} residues to be annealed in the double-stranded oligonucleotide, and wherein the sequence 5′-GTTGTAAC-3′ (SEQ ID NO: 3) of said first strand is annealed to the complementary sequence 5′-GTTACAAC-3′ (SEQ ID NO: 4) of said second strand.

The double-stranded oligonucleotide may comprise at least one additional DNA or RNA residue or analogue thereof, at either or both the 5′- and 3′-ends of either or both the first and second strands.

The double-stranded oligonucleotide may be forked.

The analogue may comprise a methylated, iodinated, brominated or biotinylated residue.

The double-stranded oligonucleotide may be derivatized to include 5′- and/or 3′-insertions that do not adversely affect its ability to bind to a Tus protein. The insertions may include the addition of mRNA and/or DNA that is to be presented or displayed.

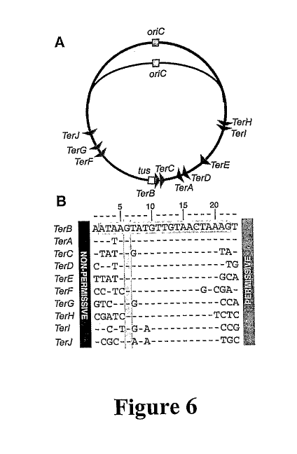

The double-stranded oligonucleotide may be readily modified by 5′- and/or 3′ insertions, deletions or substitutions, or by internal insertions, deletions or substitutions, that do not disrupt hydrogen bond formation between the central core sequence 5′-GTTGTAAC-3′ (SEQ ID NO: 3) of the first strand and the complementary sequence 5′-GTTACAAC-3′ (SEQ ID NO: 4) of the second strand, or delete the conserved cytosine that is present in the second strand of naturally-occurring Ter sites and involved in fork arrest (as shown in FIG. 6).

5′- or 3′-nucleotide substitutions relative to a naturally-occurring Ter site, or 5′- and 3′-insertions relative to the sequence of the oligonucleotides as described above, are at least about 1-10 nucleotides in length. However, longer substitutions or insertions, such as those up to about 15 or 16 or 17 or 18 or 19 or 20 nucleotides in length, are also contemplated by the present invention.

The length of an internal substitution of the sequence of the oligonucleotides as described above is restricted by the length of the nucleic acid and the requirements for both maintenance of the conserved cytosine involved in fork arrest and hydrogen bonding of the central core sequence. Accordingly, such substitutions may generally involve one or two or three or four or five or six or seven or more consecutive or spaced-apart nucleotides.

5′ or 3′- or internal substitutions may be positioned in the first strand upstream of the central core sequence 5′-GTTGTAAC-3′ (SEQ ID NO: 3).

Internal substitutions may also be positioned on the second strand downstream of the conserved cytosine residue involved in fork arrest in naturally-occurring Ter sites.

Deletions relative to the sequence of the oligonucleotides as described above may be of one or two or three nucleotides and positioned in the first strand upstream of the central core sequence 5′-GTTGTAAC-3′ (SEQ ID NO: 3).

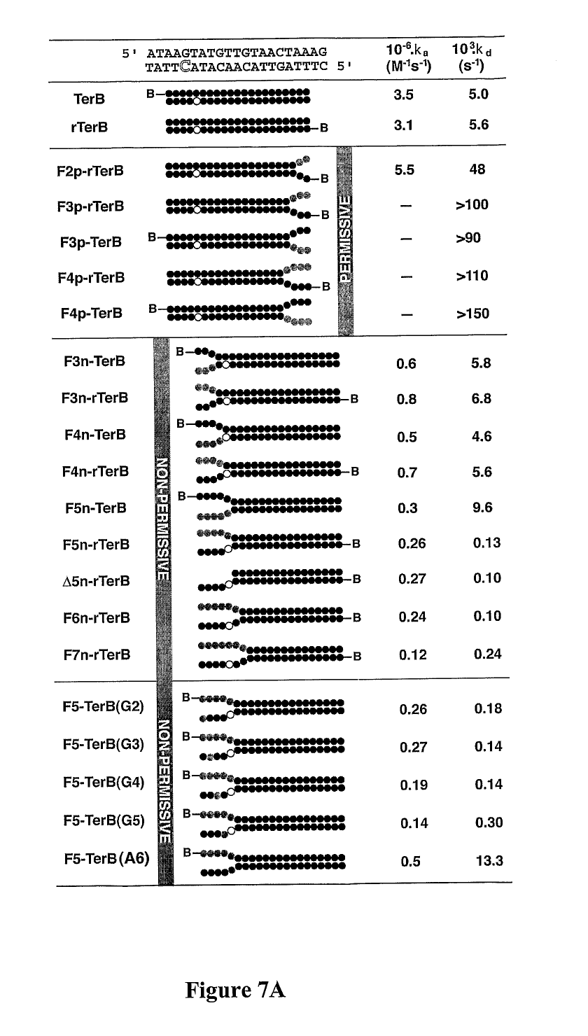

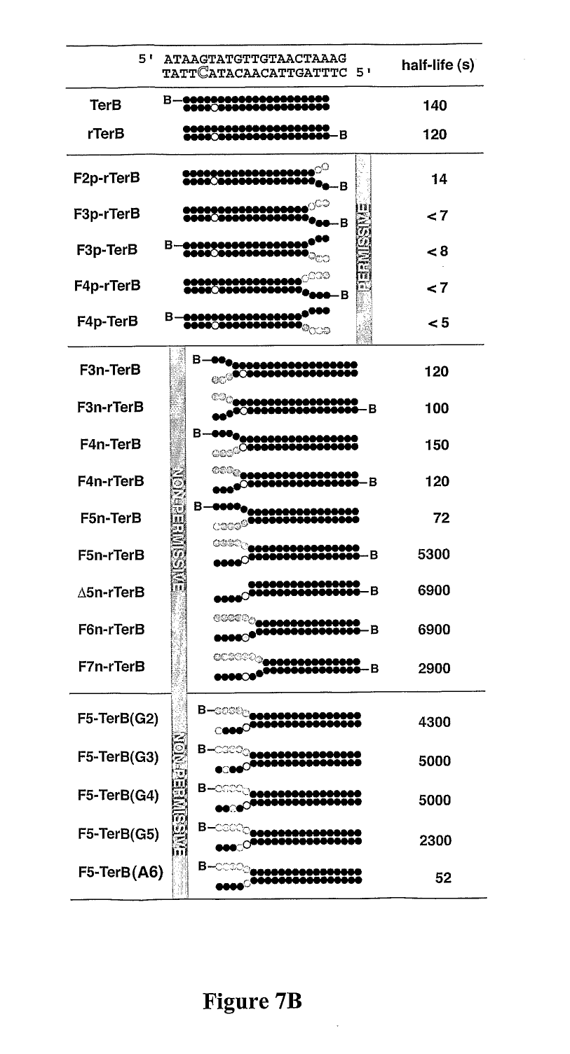

As shown in FIG. 7, mutations downstream of the central core in the first strand may, if not accompanied by upstream mutations or mutations in the opposite strand downstream of the central core, reduce the half life for dissociation from Tus, thereby producing an oligonucleotide that does not bind effectively. However, the present invention encompasses double-stranded oligonucleotides comprising substitutions or insertions in the first strand downstream of the central core, for example in combination with one or more substitutions, insertions or deletions elsewhere in the molecule relative to the sequence of the oligonucleotides as described above.

### TT-Lock Oligonucleotide Structure

The foregoing modifications may produce a forked structure downstream of a cytosine residue of the second strand that is conserved in a naturally-occurring Ter site and involved in fork arrest. Alternatively, a modification that produces a forked structure in the double-stranded oligonucleotides of the present invention may occur upstream of a naturally-occurring guanosine residue in the first strand in a naturally-occurring Ter site. If such an upstream forked structure is present, base-pairing with the other strand through this modified nucleotide residue may not occur in the double-stranded oligonucleotides. A modification that produces a forked structure in the double-stranded nucleic acid molecule may include modification of this guanosine residue on the first strand, and in particular may include one or two or three nucleotide residues downstream of the guanosine residue in the first strand.

The fork may be any length, and may comprise 1-5 or 5-10 or 10-15 or 15-20 nucleotides in length. The length of this fork may modify the rate of dissociation of the double-stranded oligonucleotide from a Tus polypeptide, such that dissociation rates may become progressively faster as the length of the fork increases, with or without simultaneous mutation of the other strand.

For example, forks produced by the addition of up to about five nucleotide residues from a naturally-occurring TerB site to the first strand sequence of the oligonucleotides as described above may exhibit half-lives for dissociation from Tus at 20° C. that are at least approximately the same as for a wild-type TerB oligonucleotide. Similarly, forks that are produced by the addition of up to about four nucleotide residues from a naturally-occurring TerB site to the second strand sequence of the oligonucleotides as described above may exhibit half-lives for dissociation from Tus at 20° C. that are at least approximately the same as for a wild-type TerB oligonucleotide. The subsequent mutation of such forks by substitution of up to about four of these additional nucleotides in the 5′-region of the first strand or the second strand may not reduce the half-life for dissociation from Tus relative to the wild-type TerB sequence. In contrast, a fork-producing mutation, for example a substitution or deletion, of five or more nucleotides positioned upstream of the central core sequence 5′-GTTGTAAC-3′ (SEQ ID NO: 3) in the first strand of native TerB, may increase the half-live of dissociation of the double-stranded oligonucleotide from a Tus polypeptide by at least about 10-fold, at least about 20-fold or at least about 50-fold relative to a wild-type TerB. Such mutations may also be combined with one or more nucleotide mutations, for example, substitutions downstream of the conserved cytosine involved in fork arrest of native TerB sites without adversely affecting half-life of Ter/Tus complex formation. It will be appreciated by the skilled artisan that a higher half-life for dissociation of the double-stranded oligonucleotide from a Tus polypeptide may be desirable for display or presentation of a molecule using the interaction between the oligonucleotide and a Tus polypeptide. This is because complexes that dissociate rapidly may be too unstable to permit operations to be performed.

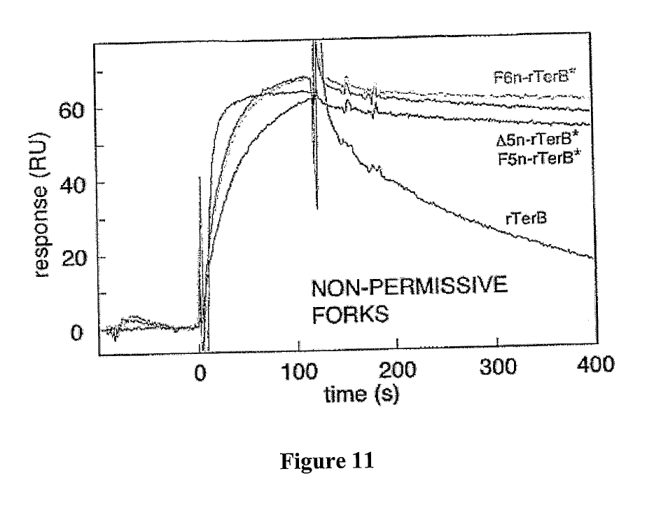

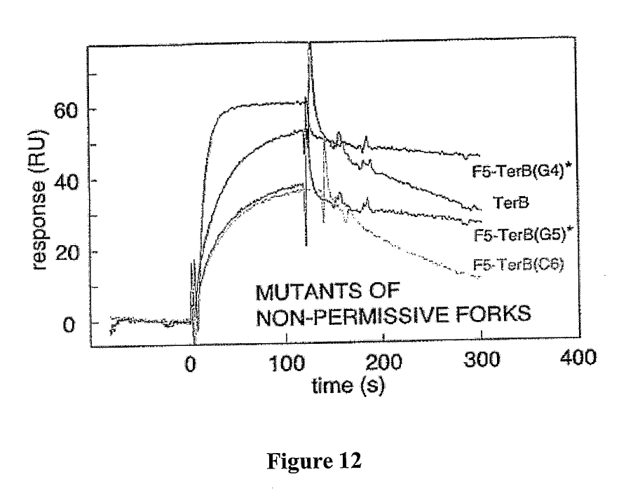

The conserved cytosine residue involved in fork arrest of a naturally-occurring Ter site (e.g, native TerB) may not be base-paired in the double-stranded oligonucleotide of the present invention, especially when it comprises a fork structure positioned upstream of the central core sequence 5′-GTTGTAAC-3′ (SEQ ID NO: 3) in the first strand. As shown in FIGS. 11 and 12, mispairing of this residue exhibits very slow dissociation rates (that is, a “locked” behaviour) and is particularly suitable for displaying or presenting any molecule.

Forked structures may be conveniently produced by synthesizing first and second strand oligonucleotides and annealing the strands, wherein the sequence upstream of the central core sequence 5′-GTTGTAAC-3′ (SEQ ID NO: 3) in the first strand may be non-complementary to a sequence downstream of a complementary central core sequence (for example in the 3′-region) of the second strand.

Alternatively, an open loop may be included upstream or downstream from the central core sequence without adversely affecting the half-life for dissociation of the double-stranded oligonucleotide from a Tus polypeptide. Such loops may comprise one or two or three or four or five or more consecutive residues. The loop may comprise and/or flank a conserved cytosine residue involved in fork arrest. A loop may be introduced into the double-stranded oligonucleotides of the invention by introducing one or more nucleotide substitutions into the first and/or second strand sequence of a naturally-occurring Ter site. For example, a loop may be produced by synthesizing first and second strand oligonucleotides and annealing the strands, wherein the upstream sequence proximal to the central core sequence 5′-GTTGTAAC-3′ (SEQ ID NO: 3) in the first strand is non-complementary to a sequence in the second strand and the upstream sequence distal thereto is complementary to a 3′-region of the second strand sequence.

### Alternative Forms of the TT-Lock Oligonucleotide

The inventors have also carried out mutagenesis of the minimum TT-Lock sequence of the double-stranded oligonucleotide sequence set forth as SEQ ID NO: 1 and SEQ ID NO: 2 to determine whether or not the ability of the oligonucleotide to capture or be captured by (i.e., the ability of the oligonucleotide to bind to) Tus protein is modified by 5′- and/or 3′-additions to one or both nucleic acid strands. The inventors found that the nucleic acid molecule of the invention is tolerant to such additions.

Accordingly, the present invention encompasses alternative forms of the TT-Lock oligonucleotide. Alternative forms of the TT-Lock oligonucleotide may comprise a modified form of the double-stranded oligonucleotide sequence set forth as SEQ ID NO: 1 and SEQ ID NO: 2 selected from the group consisting of:

(i) an oligonucleotide wherein the first strand further comprises 1 or 2 ribonucleotides, deoxyribonucleotides or analogues thereof positioned upstream of SEQ ID NO: 1 wherein said nucleotides do not form a base pair in the double-stranded oligonucleotide, for example, by virtue of not being complementary to a residue of the second strand;

(ii) an oligonucleotide wherein the second strand further comprises a ribonucleotide, deoxyribonucleotide or analogue thereof positioned downstream of SEQ ID NO: 2 wherein said nucleotide does not form a base pair in the double-stranded oligonucleotide, for example, by virtue of not being complementary to a residue of the first strand;

(iii) an oligonucleotide wherein the first strand further comprises one or more ribonucleotides, deoxyribonucleotides or analogues thereof positioned upstream and/or downstream of SEQ ID NO: 1;

(iv) an oligonucleotide wherein the second strand further comprises one or more ribonucleotides, deoxyribonucleotides or analogues thereof positioned upstream of SEQ ID NO: 2;

(v) an oligonucleotide wherein the first strand further comprises 1 or 2 ribonucleotides, deoxyribonucleotides or analogues thereof positioned upstream of SEQ ID NO: 1 wherein said nucleotides do not form a base pair in the double-stranded oligonucleotide, for example, by virtue of not being complementary to a residue on the second strand unless said ribonucleotides, deoxyribonucleotides or analogues thereof are not located at the 5′-terminus of said first strand;

(vi) an oligonucleotide wherein the first strand further comprises 1 or 2 ribonucleotides, deoxyribonucleotides or analogues thereof positioned downstream of SEQ ID NO: 2 wherein said nucleotides do not form a base pair in the double-stranded oligonucleotide, for example, by virtue of not being complementary to a residue on the first strand unless said ribonucleotides, deoxyribonucleotides or analogues thereof are not located at the 5′-terminus of said second strand; and

(vii) a combination of any one or more of (i) to (vi).

In a first embodiment of a modified form of the double-stranded oligonucleotide sequence set forth as SEQ ID NO: 1 and SEQ ID NO: 2:

(a) said first strand comprises the sequence:

(SEQ ID NO: 55)

5′-(N_{A})ₘ N_{E} N_{E} N_{B} N_{B} N_{C} R N_{D} G T T G T A A C N_{D} A
(N_{A})ₙ-3′

or an analogue or derivative of said sequence; and

(b) said second strand comprises the sequence:

(SEQ ID NO: 56)

5′-(N_{A})ₚ T N_{D} G T T A C A A C N_{D} T N_{C} C N_{B} N_{E} N_{E}
(N_{A})ₒ-3′

or an analogue or derivative of said sequence

wherein N_{A}, N_{B} and N_{E} may each be any DNA or RNA residue or analogue thereof, each of N_{A} and N_{B} is optional, subject to the proviso that when any occurrence of N_{B} is present it is not base-paired to another residue, base-pairing of each of N_{C} to another residue is optional, each of N_{D} is base-paired with another residue, each of N_{E} is optional, subject to is the proviso that if one or more of N_{E} is present it is not base-paired unless m=0 or o=0, m, n, o, p, are each an integer including zero, and said first strand and said second strand may be of equal or unequal length.

R may be A or G. R may be A.

In the double-stranded oligonucleotide, each occurrence of N_{D} on either side or flanking the central core in the first strand may be base-paired to another occurrence of N_{D} on either side or flanking the central core in the second strand, such that hybridization of the central core is not disrupted.

N_{D} may be T or A. N_{D} of the first strand may be T and N_{D} of the second strand may be A.

The occurrences of N_{C} in the first and second strands may not be base-paired, that is, they may not be complementary. Alternatively, the occurrences of N_{C} in the first and second strands may be complementary (that is, A and T, or G and C, or A and U, or analogues thereof) and base-paired to each other.

N_{C} may be T or A. N_{C} of the first strand may be T and N_{C} of the second strand may be A.

At least one occurrence of N_{A} may be absent, that is, m=0 and/or n=0 and/or o=0 and/or p=0. m may equal 0 and/or o may equal 0. m may equal 0 or o may equal 0.

At least one occurrence of N_{A} may be present and base-paired to another residue. At least one occurrence of N_{A} may be present, however not base-paired to another residue. In accordance with these embodiments, the integer “o” may have a value of up to about 20, or at least about 1-5 or 5-10 or 15-20 or a value of between 1 and 15, including values of 1 or 15.

Alternatively, or in addition, the integer “n” may have a value of up to about 20, or at least about 1-5 or 5-10 or 15-20 or a value of up to about 5, including values of 1 or 2 or 3 or 4 or 5. n may equal 3.

Alternatively, or in addition, the integer “o” may have a value of up to about 20, or at least about 1-5 or 5-10 or 15-20 or a value of between 1 and 15, including values of 1 or 15.

Alternatively, or in addition, the integer “p” may have a value of up to about 20, or about 1-5 or 5-10 or 15-20 or a value of up to about 5, including values of 1 or 2 or 3 or 4 or 5. p may equal 3.

N_{A} may be selected from the group consisting of A, C, G or T. Optionally, an occurrence of N_{A} at the 5′-terminal position of the first and/or second strand sequence may be labelled with a biotin moiety or may be a biotinylated residue.

In one embodiment, at least one occurrence of N_{B} and/or N_{E} may be absent. As these residues are internal to the sequence of the first and second strands, this means that those residues terminal to the missing N_{B} and/or N_{E}, if present, may take the position of the missing residue in the first or second strand and, as a consequence in the annealed double-stranded nucleic acid. For example, none or one or both occurrences of N_{B} and/or none or one or both occurrences of N_{E} may be present in the first strand.

Accordingly, the first strand may comprise a sequence selected from the group consisting of:

(SEQ ID NO: 61)

| (i) | 5′-(N_{A})ₘ N_{c} A N_{D} G T T G T A A C N_{D} A (N_{A})ₙ- |
|---|---|
| | 3′; |

(SEQ ID NO 62)

| | |
|---|---|
| (ii) | 5′-(N_{A})ₘ N_{E} N_{C} A N_{D} G T T G T A A C N_{D} A |
| | (N_{A})ₙ-3′; |

(SEQ ID NO 63)

| | |
|---|---|
| (iii) | 5′-(N_{A})ₘ N_{E} N_{E} N_{C} A N_{D} G T T G T A A G N_{D} A |
| | (N_{A})ₙ-3′; |

(SEQ ID NO 64)

| | |
|---|---|
| (iv) | 5′-(N_{A})ₘ N_{B} N_{C} A N_{D} G T T G T A A C N_{D} A |
| | (N_{A})ₙ-3′; |

(SEQ ID NO: 65)

| | |
|---|---|
| (v) | 5′-(N_{A})ₘ N_{E} N_{B} N_{C} A N_{D} G T T G T A A C N_{D} |
| | A (N_{A})ₙ-3′; |

(SEQ ID NO: 66)

| | |
|---|---|
| (vi) | 5′-(N_{A})ₘ N_{E} N_{E} N_{B} N_{C} A N_{D} G T T G T A A C N_{D} |
| | A (N_{A})ₙ-3′; |

(SEQ ID NO: 67)

| | |
|---|---|
| (vii) | 5′-(N_{A})ₘ N_{B} N_{B} N_{C} A N_{D} G T T G T A A C N_{D} A |
| | (N_{A})ₙ-3′; |
| and | |

(SEQ ID NO: 68)

| | |
|---|---|
| (viii) | 5′-(N_{A})ₘ N_{E} N_{B} N_{B} N_{C} A N_{D} G T T G T A A C N_{D} |
| | A (N_{A})ₙ-3′. |

Similarly, N_{B} may be absent or present and/or none or one or both occurrences of N_{E} may be present in the second strand.

Accordingly, the second strand may comprise a sequence selected from the group consisting of:

(SEQ ID NO: 69)

| | |
|---|---|
| (i) | 5′-(N_{A})ₚ T N_{D} G T T A C A A C N_{D} T N_{C} C |
| | (N_{A})ₒ-3′; |

(SEQ ID NO: 70)

| | |
|---|---|
| (ii) | 5′-(N_{A})ₚ T N_{D} G T T A C A A C N_{D} T N_{C} C N_{E} |
| | (N_{A})ₒ-3′; |

(SEQ ID NO: 71)

| | |
|---|---|
| (iii) | 5′-(N_{A})ₚ T N_{D} G T T A C A A C N_{D} T N_{C} C N_{E} |
| | N_{E} (N_{A})ₒ-3′; |

(SEQ ID NO: 72)

| | |
|---|---|
| (iv) | 5′-(N_{A})ₚ T N_{D} G T T A C A A C N_{D} T N_{C} C N_{B} |
| | (N_{A})ₒ-3′; |

(SEQ ID NO: 73)

| | |
|---|---|
| (v) | 5′-(N_{A})ₚ T N_{D} G T T A C A A C N_{D} T N_{C} C N_{B} |
| | N_{E} (N_{A})ₒ-3′; |
| and | |

(SEQ ID NO: 74)

| | |
|---|---|
| (vi) | 5′-(N_{A})ₚ T N_{D} G T T A C A A C N_{D} T N_{C} C N_{B} |
| | N_{E} N_{E} (N_{A})ₒ-3′. |

In one embodiment, “m” may equal 0 and/or “o” may equal 0 and at least one occurrence of N_{E} may be present and base-paired to another residue in the double-stranded nucleic acid.

In another embodiment, “m” may equal 0 and/or “o” may equal 0 and at least one occurrence of N_{E} may be present and not base-paired to another residue in the double-stranded nucleic acid.

Such embodiments encompass situations wherein, of the maximum four occurrences of N_{E} in the double-stranded nucleic acid, one or two or three or four occurrences is present and/or one or two or three or four of those potential occurrences is base-paired.

N_{B} and N_{E} may be selected from the group consisting of A, C, G or T.

Accordingly, in a second embodiment of a modified form of the double-stranded oligonucleotide sequence set forth as SEQ ID NO: 1 and SEQ ID NO: 2, said first strand may comprise the sequence:

(SEQ ID NO: 57)

5′-(N_{A})₁₋₁₅ N_{E} N_{E} N_{B} N_{B} N_{C} R N_{D} G T T G T A A C N_{D}
A (N_{A})₃-3′

or an analogue or derivative of said sequence.

In a third embodiment of a modified form of the double-stranded oligonucleotide sequence set forth as SEQ ID NO: 1 and SEQ ID NO: 2, said first strand may comprise the sequence:

(SEQ ID NO: 58)

5′-(N_{A})₁₋₁₅ N_{E} N_{E} N_{B} N_{B} N_{C} R T G T T G T A A C T A
A A G-3′

or an analogue or derivative of said sequence.

N_{C} may be selected from the group consisting of G, C, T and analogues thereof.

Exemplary first-strand sequences within the scope of the second and third embodiments as set out above are set forth in Table 1.

TABLE 1
Exemplary first-strand sequences

| | | |
|---|---|---|
| Oligo Name | Sequence | SEQ ID NO: |

| | | |
|---|---|---|
| Δ5n | 5′-TATGTTGTAACTAAAG-3′ | SEQ ID NO: 31 |
| F5n | 5′-GGGCTATGTTGTAACTAAAG-3′ | SEQ ID NO: 30 |
| F6n | 5′-GGGCGATGTTGTAACTAAAG-3′ | SEQ ID NO: 32 |
| F7n | 5′-GGGCGGTGTTGTAACTAAAG-3′ | SEQ ID NO: 33 |
| 1 mismatch | 5′-GCAGCCAGCTCCGAATAATTATGTTGTAACTAAAG-3′ | SEQ ID NO: 49 |
| 2 mismatch | 5′-GCAGCCAGCTCCGAATACTTATGTTGTAACTAAAG-3′ | SEQ ID NO: 50 |
| 3 mismatch | 5′-GCAGCCAGCTCCGAATCCTTATGTTGTAACTAAAG-3′ | SEQ ID NO: 51 |
| 4 mismatch | 5′-GCAGCCAGCTCCGAAACCTTATGTTGTAACTAAAG-3′ | SEQ ID NO: 52 |
| 5 mismatch | 5′-GCAGCCAGCTCCGAAACCTCATGTTGTAACTAAAG-3′ | SEQ ID NO: 53 |
| Flipped C6 | 5′-GCAGCCAGCTCCGAATAATATGTTGTAACTAAAG-3′ | SEQ ID NO: 54 |
| TerB/rTerB | 5′-ATAAGTATGTTGTAACTAAAG-3′ | SEQ ID NO: 16, 18 |
| Ext-TerB | 5′-GCAGCCAGCTCCGAATAAGTATGTTGTAACTAAAG-3′ | SEQ ID NO: 47 |

In a fourth embodiment of a modified form of the double-stranded oligonucleotide sequence set forth as SEQ ID NO: 1 and SEQ ID NO: 2, said second strand may comprise the sequence:

(SEQ ID NO: 59)

5′-(N_{A})₃ T A G T T A C A A C A T A C N_{B} N_{E} N_{E}
(N_{A})₁₋₁₅-3′

or an analogue or derivative of said sequence.

In a fifth embodiment of a modified form of the double-stranded oligonucleotide sequence set forth as SEQ ID NO: 1 and SEQ ID NO: 2, said second strand may comprise the sequence:

(SEQ ID NO: 60)

5′-C T T T A G T T A C A A C A T A C N_{B} N_{E} N_{E}
(N_{A})₁₋₁₅-3′

or an analogue or derivative of said sequence.

Exemplary second-strand sequences within the scope of the fourth and fifth embodiments as set out above are set forth in Table 2.

TABLE 2
Exemplary second-strand sequences

| | | |
|---|---|---|
| Oligo Name | Sequence | SEQ ID NO: |

| | | |
|---|---|---|
| TerB/rTerB | 5′-CTTTAGTTACAACATACTTAT-3′ | SEQ ID NO: 17, 19 |
| Δ3N-TerB | 5′-CTTTAGTTACAACATACACT-3′ | SEQ ID NO: 42 |
| F3n-TerB | 5′-CTTTAGTTACAACATACTCCC-3′ | SEQ ID NO: 25 |
| F4n-TerB | 5′-CTTTAGTTACAACATACGCCC-3′ | SEQ ID NO: 27 |
| TerB(G2) | 5′-CTTTAGTTACAACATACTTAG-3′ | SEQ ID NO: 35 |
| TerB(G3) | 5′-CTTTAGTTACAACATACTTTT-3′ | SEQ ID NO: 36 |
| TerB(G4) | 5′-CTTTAGTTACAACATACTGAT-3′ | SEQ ID NO: 37 |
| TerB(G5) | 5′-CTTTAGTTACAACATACGTAT-3′ | SEQ ID NO: 38 |
| Single O/H C | 5′-CTTTAGTTACAACATAC-3′ | SEQ ID NO: 44 |
| Bromo-Lock | 5′-C T T BrdU A G T T A C A A | SEQ ID NO: 45 |
| | C A BrdU A C T T A T-3′ | |
| Iodo-Lock | 5′-C T T IdU A G T T A C A A C | SEQ ID NO: 46 |
| | A IdU A C T T A T-3′ | |
| Ext-TerB | 5′-CTTTAGTTACAACATACTTATTCGGAG | SEQ ID NO: 48 |
| | CTGGCTGC-3′ | |

The present invention encompasses any combination of the first strand and second strand sequences set forth in Tables 1 and 2, the only exceptions being either a combination that produces a naturally-occurring or native TerB site, or other naturally-occurring Ter sites, which are to be excluded. Such excluded sequences are produced, for example, by combination of a first strand consisting of SEQ ID NO: 16 or 18 with SEQ ID NO: 17 or 19, or by combination of SEQ ID NOs: 47 and 48. Such native molecules do not fall within the scope of any of the first to fifth embodiments described herein by virtue of the requirements therein for N_{B} to not be base-paired to another residue and for one or more of N_{E} not to be base-paired unless m=0 or o=0.

### Conjugation of an Oligonucleotide to a Polypeptide or Protein

In one embodiment, the double-stranded oligonucleotides of the invention or a first or second strand thereof may be conjugated to another molecule of interest such as a peptide, polypeptide, protein, antibody or antibody fragment.

The double-stranded oligonucleotides may be derivatized to include 5′- and/or 3′-insertions that do not adversely affect its ability to bind to a Tus polypeptide. The insertions may include the addition of mRNA and/or DNA that is to be presented or displayed.

In another embodiment, the double-stranded oligonucleotides as described above may be bound to one or more proteinaceous molecules, nucleic acid molecules, or small molecules. The binding may be covalent or non-covalent. Non-covalent binding of the oligonucleotides may be to a Tus polypeptide (e.g., SEQ ID NO; 5) having TerB-binding activity such as, for example, a fusion polypeptide comprising Tus and a polypeptide to be displayed on a microwell or microarray surface or on the surface of a cell, phage, virus or in vitro. Covalent linkages may be between the double-stranded oligonucleotides and a non-Tus proteinaceous molecule, nucleic acid molecule, or small molecule.

In a further embodiment, the double-stranded oligonucleotide as described above may be bound to:

(i) a Tus polypeptide (e.g., SEQ ID NO; 5) having TerB-binding activity; and

(ii) a proteinaceous molecule, nucleic acid molecule, or small molecule.

The double-stranded oligonucleotide derivative may therefore further comprise DNA or RNA to be displayed on a microwell or microarray surface or on the surface of a cell, phage, virus or in vitro. The Tus polypeptide derivative may be a fusion polypeptide comprising Tus and a polypeptide to be displayed on a microwell or microarray surface or on the surface of a cell, phage, virus or in vitro.

It will also be apparent from the disclosure herein that the double-stranded oligonucleotides of the present invention may be particularly useful for presenting or displaying one or more other molecules to which it can be conjugated or covalently attached during synthesis or post-synthesis.

Accordingly, the present invention also provides a conjugate comprising the double-stranded oligonucleotides as described herein and another molecule, for example, a nucleic acid, polypeptide or small molecule.

In a further embodiment, the double-stranded oligonucleotides bound as described above are used for presentation or display. For example, a Tus polypeptide, fragment or derivative thereof having TerB binding activity may be conjugated to a peptide, polypeptide, antibody or fragment thereof, or a small molecule, and presented in combination with the double-stranded oligonucleotide for assay purposes. As will be known to the skilled artisan, the peptide, polypeptide or antibody fragment may be produced by recombinant means as an in-frame fusion with a Tus polypeptide. Alternatively, a peptide, polypeptide, antibody or fragment thereof, or a small molecule may be conjugated to a Tus polypeptide by chemical means. Accordingly, the present invention also encompasses a conjugate comprising a Tus polypeptide and another molecule. The conjugate may be a Tus polypeptide derivative.

It is also within the scope of the present invention to use a conjugate comprising mRNA encoding a Tus protein fused in the same reading frame to mRNA encoding a second polypeptide.

Methods for conjugating a nucleic acid to a peptide, polypeptide or protein are known in the art and include, for example, covalent or non-covalent conjugation. For example, a non-covalent interaction, such as an ionic bond, a hydrophobic interaction, a hydrogen bond and/or a van der Waals attraction may be used to produce a nucleic acid:protein conjugate. Such a non-covalent interaction may be produced, for example, using an ionic interaction involving a modified nucleic acid and residues within the peptide, polypeptide or protein, such as charged amino acids, or by using of a linker comprising charged residues that interacts with both the nucleic acid and the peptide, polypeptide or protein. For example, non-covalent conjugation may occur between a generally negatively-charged modified nucleic acid and positively-charged amino acid residues of a peptide, polypeptide or protein, for example, polylysine and/or polyarginine residues.

Alternatively, a non-covalent conjugation between a nucleic acid and a peptide, polypeptide or protein may be produced using a DNA binding motif of a molecule that interacts with nucleic acid as a natural ligand. For example, such DNA binding motifs may be found in transcription factors and anti-DNA antibodies. By fusing the nucleic acid to the binding site of the DNA binding motif, and the peptide, polypeptide or protein to the DNA binding motif a non-covalent interaction may be produced.

In another embodiment, a covalent interaction may used to produce a nucleic acid:protein conjugate. A general method to form a protein:nucleic acid conjugate involves coupling a linker compound to an oligonucleotide sequence during synthesis. If necessary a functional group on the linker and/or on the oligonucleotide may then be deprotected, for example, by ammonia or hydroxide treatment. A suitable method of deprotection will be apparent to the skilled artisan. The linker may then be activated and the modified oligonucleotide reacted with a peptide, polypeptide or protein to form a covalent linkage. Suitable examples of this method are described, for example, in Agrawal et al. Nucleic Acids Res. 14:6227-6245, 1986 or Connolly Nucl. Acids Res. 13:4485-4502, 1985; or U.S. Pat. Nos. 4,849,513; 5,015,733; 5,118,800; and 5,118,802.

In a specific example of this method, a linker containing a carbomethoxy group may be coupled to a resin-bound oligonucleotide in a DNA synthesizer. After simultaneous deprotection (should the oligonucleotide contain any protecting groups), ester hydrolysis and resin removal, the newly formed carboxylic acid may be activated with a carbodiimide, such as, for example, 1-ethyl-3-(dimethylaminopropylcarbodiimide) (EDAC), N-hydroxysuccinimide, N-hydroxybenzotriazole, or tetrafluorophenol may be added to form an active ester in situ. This activated carboxyl group may then be reacted with a peptide, polypeptide or protein to form a covalent oligonucleotide-linking group-peptide, -polypeptide or -protein conjugate.

In another example, Zuckermann et al., Nucl. Acids Res. 15: 5305-5321 describe a method for conjugating a peptide, polypeptide or protein to the 3′ end of a nucleic acid. The method involves the incorporation of a sulfhydryl group into the 3′-nucleotide or nucleoside-support linkage as a disulfide bond, prior to automated oligonucleotide synthesis. The approach described avoids complications due to functionalities present in the final oligonucleotide. The oligonucleotide may be synthesized from the thiolated 3′-terminal nucleoside (or nucleotide) using standard solid phase phosphotriester or phosphoramidite chemistry, deprotected by conventional methods, treated with dithiothreitol (DTT), and purified by reverse phase chromatography. The thiolated oligonucleotide may then be activated with 2,2′-dithiodipyridine and cross-linked to a thiol containing peptide, polypeptide or protein. Alternatively, the 3′-thiol-containing oligonucleotide may be derivatized with an electrophile such as an α-haloacetyl or maleimidyl group conjugated to the peptide, polypeptide or protein.

Alternatively, a peptide, polypeptide or protein may be conjugated to the 3′-end of a nucleic acid through solid support chemistry. For example, the nucleic acid may be added to a polypeptide portion that has been pre-synthesized on a support as described in Haralambidis et al. Nucleic Acids Res. 18:493-499, 1990 or Haralambidis et al. Nucleic Acids Res. 18:501-505, 1990. These methods may involve the synthesis of a peptide or polypeptide of interest on a solid support, for example, using Boc chemistry. At the terminus of the peptide or polypeptide polyamide, synthesis may be performed and the terminal amino group converted to a protected primary aliphatic hydroxy group by reaction with alpha, omega-hydroxycarboxylic acid derivatives. Oligonucleotide synthesis may then be performed using phosphoramidite chemistry

In another embodiment, the nucleic acid may be synthesized such that it is connected to a solid support through a cleavable linker (a modified nucleic acid) extending from the 3′ terminus. Upon chemical cleavage of the modified nucleic acid from the support, a terminal thiol group may be left at the 3′-end of the oligonucleotide (Corey et al. Science 238:1401-1403, 1987) or a terminal amine group left at the 3′-end of the oligonucleotide (Nelson et al. Nucleic Acids Res. 17:1781-1794, 1989). Conjugation of the amino-modified nucleic acid to amino groups of a peptide, polypeptide or protein may then be performed as described in Benoit et al. Neuromethods 6:43-72, 1987. Conjugation of the thiol-modified modified oligonucleotide to carboxyl groups of the peptide may be performed as described in Sinah et al. 1991, Oligonucleotide Analogues. A Practical Approach, IRL Press.

Compounds may also be attached to the 3′ end of oligomers, as described by Asseline et al., Tet. Lett. 30:2521, 1989. This method utilizes 2,2′-dithioethanol attached to a solid support to displace diisopropylamine from a 3′ phosphonate bearing an acridine moiety that may be subsequently deleted after oxidation of the phosphorus. Other substituents have been bound to the 3′ end of oligomers by alternate methods, including the use of polylysine (Bayard et al., Biochemistry 25:3730, 1986). Additional methods of attaching non-nucleotide compounds to oligonucleotides are discussed in U.S. Pat. Nos. 5,321,131 and 5,414,077.

In another embodiment, the peptide, polypeptide or protein may be conjugated to the 5′ end of the oligonucleotides of the invention. For example, Haralambidis et al., Nucl. Acids Res., 15: 4857-4876, 1987 describe a method for conjugating a nucleic acid to a peptide, polypeptide or protein. This method utilises a C-5 substituted deoxyuridine nucleoside in the production of an oligonucleotide. The substituent carries a masked primary aliphatic amino group. This key intermediate may then be functionalized at its C-5 substituent to give nucleosides with longer C-5 arms. The resulting oligonucleotide may then readily be reacted with a peptide, polypeptide or protein of interest to produce a conjugate.

In another embodiment, a nucleic acid may be produced that is linked to a moiety comprising a free amine group. The amine may then be derivatized with a maleimide- or haloacetyl-containing heterobifunctional agent, such as N-succinimidyloxy-4(N-maleimido-methyl)-cyclohexane-1 carboxylate (SMCC) or iodoacetic anhydride, and then conjugated to a thiol group on a peptide, polypeptide or protein. Alternatively, the amine functional group may be reacted with succinic anhydride, with the resultant free carboxylic acid group subsequently being coupled to an amine group on the peptide, polypeptide or protein using carbodiimide.

In a further alternative embodiment, the amine functional group may be reacted with a thiol-containing heterobifunctional reagent, such as iminothiolane or succinimidyloxy-3-2 (2-pyridyldithio) propionate (SPDP), followed by a treatment with a reducing agent, such as β-mercaptoethanol or dithiothreitol (DTT). The resultant free thiol group may be reacted with a maleimide or haloacetyl derivative of a peptide, polypeptide or protein. This derivatization of the peptide, polypeptide or protein may be accomplished, for example, via reaction with SMCC, iodoacetic anhydride or N-succinimidyloxy-(4-iodoacetyl) aminobenzoate (SIAB) under neutral or slightly alkaline conditions.

In another embodiment, a disulfide-bonded conjugate may be produced using an unreduced SPDP-oligonucleotide derivative as described together with a thiol-containing peptide, polypeptide or protein. Should the peptide, polypeptide or protein not contain a native thiol, the peptide, polypeptide or protein may be derivatized with iminothiolane or SPDP, followed by reduction with DTT or β-mercaptoethanol, or via DTT-mediated reduction of native disulfides.

Alternative methods for linking compounds, such as proteins, labels, small molecules, oligonucleotides and other chemical entities, to nucleotides are known in the art. For example, substituents may be attached to the 5′ end of a preconstructed oligonucleotide using amidite or H-phosphonate chemistry, as described by Ogilvie, et al., Pure and Appl Chem 59:325, 1987, and by Froehler, Nucl. Acids Res 14:5399, 1986.

Accordingly, the present invention encompasses a method for the production of a conjugate comprising an oligonucleotide of the invention and a peptide, polypeptide or protein, wherein said method comprises:

(i) producing or synthesising said oligonucleotide bound to an agent capable of forming a bond with a peptide, polypeptide or protein; and

(ii) contacting the oligonucleotide with the peptide, polypeptide or protein for a time and under conditions sufficient for a bond to form between the agent and the peptide, polypeptide or protein.

The present invention further provides a method for the production of a conjugate comprising a nucleic acid and a Tus polypeptide having Ter-binding activity, wherein said method comprises:

(i) producing or synthesising said oligonucleotide bound to an agent capable of forming a bond with a peptide, polypeptide or protein; and

(ii) contacting the oligonucleotide with the Tus polypeptide for a time and under conditions sufficient for a bond to form between the agent and the peptide, polypeptide or protein.

In one embodiment, the method additionally comprises isolating the conjugated oligonucleotide and peptide, polypeptide or protein, for example, by using reverse phase chromatography, precipitation or affinity chromatography.

### Conjugation of an Oligonucleotide to a Non-Proteinaceous Compound

In another embodiment, the oligonucleotides of the present invention are conjugated to a non-proteinaceous molecule such as a lipid, oligosaccharide or small molecule.

Several of the methods described above may be also useful for conjugating a nucleic acid of the invention to such non-proteinaceous compounds. For example, production of a nucleic acid linked to a moiety comprising a free amine group may facilitate the use of a chemical cross-linking agent that may be useful for linking the oligonucleotides to any of a variety of compounds.

An oligonucleotide of the invention may be linked to a lipid using a method known in the art, such as, for example, synthesis of oligonucleotide-phospholipid conjugates (Yanagawa et al. Nucleic Acids Symp. Ser. 19:189-192, 1988), oligonucleotide-fatty acid conjugates (Grabarek et al. Anal. Biochem. 185:131-135, 1990; and Staros et al. Anal. Biochem. 156:220-222, 1986), and oligonucleotide-sterol is conjugates (Boujrad et al. Proc. Natl. Acad. Sci. USA 90:5728-5731, 1993).

The linkage of a nucleic acid of the invention to an oligosaccharide may be achieved using a method, such as, for example, the synthesis of oligonucleotide-oligosaccharide conjugates, wherein the oligosaccharide may be a moiety of an immunoglobulin (as described in O'Shannessy et al. J. Applied Biochem. 7:347-355, 1985).

### Conjugation of an Oligonucleotide to Another Nucleic Acid

In yet another embodiment, the oligonucleotides of the invention are conjugated to a nucleic acid of interest. In this regard, the nucleic acid of interest may comprise DNA, RNA, a derivative of DNA, a derivative of RNA or a combination thereof. Furthermore, the nucleic acid of interest may be, for example, single stranded, duplex or triplex nucleic acid.

Methods for the production of such conjugated nucleic acids are known in the art and described, for example, in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001). For example, a nucleic acid molecule comprising the nucleic acid of the invention and a nucleic acid of interest may be synthesized. Methods of oligonucleotide synthesis are known in the art and described, for example, in Gait (Ed) (In: Oligonucleotide Synthesis: A Practical Approach, IRL Press, Oxford, 1984). In this regard, the nucleic acid synthesized may comprise any combination of nucleotides (e.g., DNA or RNA) and/or nucleotide analogues or derivatives.

Alternatively, a single nucleic acid molecule comprising the oligonucleotides of the invention and a nucleic acid of interest may be produced using recombinant means, such as, for example, splice overlap extension. For example, an oligonucleotide of the invention may be amplified using, for example, PCR, in which one of the primers used in the reaction comprises a sequence that is capable of hybridizing to the nucleic acid of interest. By using the resulting amplification product in a further PCR reaction to amplify the nucleic acid of interest, a single nucleic acid molecule comprising both the to oligonucleotide of the invention and the nucleic acid of interest may be produced.

The method of Tian et al., (Nature 432: 1050-1054, 2004) may be particularly useful for synthesising long strands of nucleic acid. This method essentially involves synthesizing a plurality of oligonucleotides that span the sequence of the nucleic acid to be produced (for example, a nucleic acid of the invention linked to a nucleic acid of interest), wherein the oligonucleotides may be synthesised on a microchip. Each oligonucleotide may comprise a restriction endonuclease site to thereby facilitate its release from the microchip. By releasing the oligonucleotides from the chip and using them in a PCR reaction (i.e., splice overlap extension) a single nucleic acid molecule may be produced.

In a further embodiment, a conjugate comprising double stranded DNA or RNA or a double stranded DNA/RNA conjugate may be produced using a DNA ligase, such as, for example, a T4 DNA ligase (as available, for example, from New England Biolabs). Such an enzyme may catalyze the formation of a phosphodiester bond between juxtaposed 5′ phosphate and 3′ hydroxyl termini in duplex DNA or RNA. Suitable methods for the ligation of DNA and/or RNA molecules using a DNA ligase are known in the art and/or described in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001).

In one embodiment, a conjugate comprising a single stranded DNA or RNA and a nucleic acid of the invention (whether single or double stranded) may be produced using an RNA ligase, such as, for example T4 RNA ligase (as available from New England Biolabs). An RNA ligase may catalyze ligation of a 5′ phosphoryl-terminated nucleic acid donor to a 3′ hydroxyl-terminated nucleic acid acceptor through the formation of a 3′-5′ phosphodiester bond, with hydrolysis of ATP to AMP and PPᵢ.

In a further embodiment, a nucleic acid conjugate may be produced using a crosslinking reagent attached to one of the nucleic acids. Any crosslinking agent capable of covalently attaching two oligonucleotides may be used, for example, psoralen. Psoralen is a photoactivated crosslinking molecule with a rigid, flat structure that readily intercalates within a dsDNA or dsRNA double helix, preferably between an AT sequence. Both the furan and pyrone functional groups of the psoralen compound may be photolyzed with long wavelength UV light (365 nm) to form covalent bonds with particular nucleotide bases. The furan side is 4 times more reactive than the pyrone side and overwhelmingly favours reacting with T nucleotides. The furan and pyrone groups also both show reactivity with C and U nucleotides. Psoralen, psoralen derivatives and special phosphoramidites with 5′ psoralen linkers are commercially available (Glen Research). Using such a compound, a nucleic acid conjugate may be produced by contacting a psoralen linked nucleic acid with another nucleic acid for a time and under conditions sufficient for a covalent bond to form (e.g., as described supra). A suitable is method for conjugating nucleic acids using psoralen is described, for example, in Kessler (1992) “Nonradioactive labeling methods for nucleic acids” in Kricka (ed.) Nonisotopic DNA Probe Techniques, Academic Press; and Geoghegan et al., Bioconjug. Chem. 3:138-146, 1992.

### Analogues and Derivatives of the Double-Stranded Oligonucleotides

The present invention encompasses any analogues and derivatives of the double-stranded oligonucleotides as described herein. For example, the oligonulceotides may be derivatized to include 5′- and/or 3′-insertions that do not adversely affect its ability to bind to a Tus protein or a homologue, analogue or derivative thereof. Such insertions include the addition of mRNA and/or DNA that is to be presented or displayed.

### Analogues of Ribonucleotides and Deoxyribonucleotides

The present invention encompasses analogues of deoxyribonucleotides or ribonucleotides, for example, wherein the base is substituted for an analogous base having the same base-pairing attributes.

Analogues of a ribonucleotide or deoxyribonucleotide may comprise modifications to the phosphate and/or sugar and/or base. Modified phosphate groups may comprise non-hydrolyzable substituents, bis-nucleoside phosphates, or gamma-phosphate linkers, amongst others, or combinations thereof. Modified sugars may comprise one or more fluorescent substituents, nucleoside biphosphates, cyclic nucleotides, amino linkers, halogen or other heavy substituents (e.g., bromine, fluorine, chlorine, iodine, astatine), arabinose, amongst others, or combinations thereof. Modified bases may comprise one or more uncommon bases (e.g., inosine, xanthine, hypoxanthine, ε-adenosine, ribavirin, dPTP, a 6-chloropurine substituent, a 6-mercaptopurine substituent), fluorescent substituents, thiol substituents (e.g., 6-thio-inosine-5′-triphosphate), amino linkers, halogen or other heavy substituents (e.g., bromine, fluorine, chlorine, iodine, astatine), amongst others, or combinations thereof. Caged nucleotide analogues incorporating one or more photolabile groups may also be employed. Such analogues are readily obtained from commercial sources e.g., Jena Bioscience GmbH, Loebstedter Str. 78, 07749 Jena, Germany.

Analogues may comprise alkylated (e.g., methylated), iodinated, brominated or biotinylated deoxyribonucleotides or ribonucleotide residues. Other analogues may also be used. For example, any one or more of A, C, G or T is substituted for a ribonucleotide or deoxyribonucleotide residue having the same or similar base-pairing ability and/or wherein T is substituted for an alkylated, biotinylated or halogenated ribonucleotide or deoxyribonucleotide having the same or similar base-pairing ability.

### 1. Fluorescent Analogues

Fluorescent analogues may comprise one or more compact fluorophores that are particularly useful as they show only minimal effects on protein-nucleotide interactions due to their low molecular weight. When incorporated into the TT-Lock oligonucleotide of the present invention, the resultant oligonucleotide may be useful for stopped-flow and equilibrium analysis of nucleotide-protein interactions in linetic studies, environmentally-sensitive fluorescence, fluorescence in-situ hybridization (FISH), ligand binding studies, energy transfer studies (FRET), fluorescence microscopy or X-ray crystallography, methods described, for example, by Hiratsuka (2003) Eur. J. Biochem. 270:3479; Gille et al. (2003) NS Arch. Pharmacol. 368:210; Gille et al. (2004) NS Arch. Pharmacol. 369:141; Gromadski et al. (2004) Nature Struct. & Molec. Biol. 11:316).

Exemplary substituents for such analogues may include N-methyl-anthraniloyl (i.e., mant); 4-(N-methyl-anthraniloyl)-amino (i.e., mant-amino); 4-(N-methyl-anthraniloyl)-amino)butyl (i.e., 4-(mant-amino)butyl); 6-(N-methyl-anthraniloyl)-amino)hexyl (i.e., 6-(mant-amino)hexyl); 2-(N-methyl-anthraniloyl)-amino)ethyl-carbamoyl (i.e., mant-EDA); 2′/3′-(O-Trinitrophenyl) (i.e., TNP); P³-(1-(2-nitrophenyl)-ethyl)-ester (i.e., NPE-caged substituent); methyl-7-guanosine (i.e., m⁷G) and the like.

Accordingly, exemplary fluorescent adenosine analogues suitable for such applications may include mant-ADP (2′/3′-O-(N-methyl-anthraniloyl)-adenosine-5′-diphosphate); mant-ATP (2′/3′-(N-methyl-anthraniloyl)-adenosine-5′-triphosphate); mant-N⁶-methyl-ATP (2′/3′-O—(N-Methyl-anthraniloyl)-N⁶-methyl-adenosine-5′-triphosphate); N⁶-[4-(mant-amino)]butyl-ATP (N⁶r[4-((N-methyl-anthraniloyl)-amino)]butyl-adenosine-5′-triphosphate); N⁶-[6-(mant-amino)]hexyl-ATP; 8-[4-(mant-amino)]butyl-ATP (MABA-ATP); 8-[6-(mant-amino)]hexyl-ATP (MAHA-ATP); mant-EDA-ATP (2′/3′-[(2-(N-methyl-anthraniloyl)-amino)ethyl-carbamoyl]-adenosine-5′-triphosphate); mant-dATP; 2′-mant-3′-dATP; mant-AppNHp (mant-AMPPNP); C-ATP (1,N⁶-etheno-ATP); ε-AppNHp (1,N⁶-etheno-adenosine-5′-[(β,γ)-imido]triphosphate or C-AMPPNP or 1,N⁶-etheno-AppNHp); TNP-ADP (2′/3′-(O-Trinitrophenyl)-adenosine-5′-diphosphate); and TNP-ATP (2′/3′-(O-Trinitrophenyl)-adenosine-5′-triphosphate).

Exemplary fluorescent guanosine analogues may include mant-GDP; mant-dGDP; mant-GTP; mant-dGTP; NPE-caged-mant-dGTP; mant-GppNHp (mant-GMPPNP); mant-dGppNHp (mant-dGMPPNP); mant-GTPγS; TNP-GDP; TNP-GTP; TNP-GppNHp (TNP-GMPPNP); ant-GTP; ant-m⁷GMP; ant-m⁷GDP; ant-m⁷GTP; and 2′-mant-3′-dGTP.

Exemplary fluorescent uridine or cytidine analogues may be 2′/3′-(O-Trinitrophenyl)-uridine-5′-triphosphate (TNP-UTP) and 2′/3′-(O-Trinitrophenyl)-cytidine-5′-triphosphate (TNP-CTP), respectively.

Exemplary fluorescent analogues of xanthine (X) or inosine (I) amy include mant-XDP; mant-XTP; mant-XppNHp (mant-XMPPNP); and mant-ITPγS.

### 2. Non-Hydrolyzable Analogues

Exemplary non-hydrolyzable adenosine analogues may include ApCp (AMPCP); ApCpp (AMPCPP); AppCp (AMPPCP); AppNHp (AMPPNP); ATPαS; dATPαS; ATPγS; mant-AppNHp (mant-AMPPNP); NPE-caged-AppNHp (NPE-caged-AMPPNP); EDA-AppNHp (EDA-AMPPNP); biotin-EDA-AppNHp; (biotin-EDA-AMPPNP); 0-methylene-APS; ε-AppNHp (ε-AMPPNP or 1,N⁶-etheno-AppNHp); and AppNH₂ (AMPPN).

Exemplary non-hydrolyzable analogues of cytidine may include dCTPαS.

Exemplary non-hydrolyzable guanosine analogues may include GpCp (GMPCP); GpCpp (GMPCPP); NPE-caged-GpCpp (NPE-caged-GMPCPP); GppCp (GMPPCP); GppNHp (GMPPNP); GDPβS; GTPαS; dGTPαS; GTPγS; mant-GppNHp (mant-GMPPNP); mant-dGppNHp (mant-dGMPPNP); mant-GTPγS; 6-thio-GpCp (6-thio-GMPCP); 6-thio-GppCp (6-thio-GMPPCP); 6-thio-GppNHp (6-thio-GMPPNP); and TNP-GppNHp (TNP-GMPPNP).

Exemplary non-hydrolyzable analogues of thymidine may include dTTPαS.

Exemplary non-hydrolyzable analogues of uridine may include UTPαS; UppNHp (UMPPNP); UTPγS; dUpNHp (dUMPNP); and dUpNHpp (dUMPNPP).

### 3. Halogenated Analogues

Exemplary halogenated analogues of adenosine may include 2′I-ADP; 2′Br-ADP; 8I-ADP; 8Br-ADP; 2′I-ATP; 2′Br-ATP; 8I-ATP; 8Br-ATP; 2′I-AppNHp (2′I-AMPPNP); 2′Br-AppNHp (2′Br-AMPPNP); 81-AppNHp (8I-AMPPNP); 8Br-AppNHp (8Br-AMPPNP); 8Br-cAMP; and 8Br-dATP.

Exemplary halogenated cytidine analogues may include 51-dCTP; 5Br—CTP; 5Br-UMP; 5Br-dCMP; 5Br-dCDP; and 5Br-dCTP.

Exemplary halogenated guanosine analogues may include 8I-GDP; 8Br-GDP; 8I-GTP; 8Br-GTP; 8I-GppNHp (8I-GMPPNP); and 8Br-GppNHp (8Br-GMPPNP).

Exemplary halogenated uridine analogues may include 51-dUMP; 5I-UTP; 5I -dUTP (5′IdU); 5Br-UTP; 5Br-dUDP (5′BrdU); 5Br-dUTP; and 5F-UTP.

Exemplary halogenated thymidine analogues may include 5I-dUMP; SI-UTP; SI-dUTP (5′IdU); 5Br-UTP; 5Br-dUDP (5′BrdU); 5Br-dUTP; and 5F-UTP.

Exemplary non-hydrolyzable analogues of xanthine or inosine may include XppCp; (XMPPCP); XppNHp (XMPPNP); mant-XppNHp (mant-XMPPNP); NPE-caged-XppNHp (NPE-caged-XMPPNP); XTPγS; IppNHp (MPPNP); ITPγS; and mant-ITPγS.

### 4. Amine-Labeled Analogues

Exemplary amine-labeled analogues of adenosine may include N⁶-(4-amino)butyl-ATP; N⁶-(6-amino)hexyl-ATP; 8-[(4-amino)butyl]-amino-ATP; 8-[(6-amino)hexyl]-amino-ATP; EDA-ADP; EDA-ATP; EDA-AppNHp (EDA-AMPPNP); >aminophenyl-ATP; γ-aminohexyl-ATP; γ-aminooctyl-ATP; γ-aminoethyl-AppNHp (>aminoethyl-AMPPNP); 8-[(6-amino)hexyl]-amino-adenosine-2′,5′-bisphosphate; and 8-[(6-amino)hexyl]-amino-adenosine-3′,5′-bisphosphate.

Exemplary amine-labeled guanosine analogues may include γ-aminohexyl-GTP; γ-aminooctyl-GTP; EDA-GTP; γ-aminohexyl-m⁷GTP; EDA-m⁷GTP; and EDA-m⁷GDP.

### 5. Thiol Analogues

Exemplary thiol guanosine analogues may include 6-thio-GTP; 6-thio-GpCp (6-thio-GMPCP); 6-thio-GppCp (6-thio-GMPPCP); 6-thio-GppNHp (6-thio-GMPPNP); 6-methylthio-GMP; 6-methylthio-GDP; 6-methylthio-GTP; 6-thio-GMP; and 6-thio-GDP.

Exemplary thiol inosine analogues may include 6-methylthio-IMP; 6-methylthio-IDP; 6-methylthio-ITP; and 6-mercaptopurine-riboside-5′-triphosphate (6-thio-inosine-5′-triphosphate).

### 6. Biotinylated Analogues

Exemplary biotinylated nucleotide analogues may include biotin-EDA-AppNHp; (biotin-EDA-AMPPNP); biotin-EDA-ATP; and biotin-EDA-AppNHp (biotin-EDA-AMPPNP).

Exemplary biotinylated uridine analogues may include biotin-XX-UTP.

### 7. 2′-Deoxyuridine Analogues

Exemplary 2′-deoxyuridine analogues may include dUDP; 5Br-dUDP; dUTP; 5Br-dUTP; dUpNHp (dUMPNP); dUpNHpp (dUMPNPP); 5I-dUTP; aminoallyl-dUpCp (aminoallyl-dUMPCP); and aminoallyl-dUpCpp (aminoallyl-dUMPCPP).

### 8. Other Suitable Analogues

Other suitable adenosine analogues may include f-methylene-APS; biotin-EDA-ATP; biotin-EDA-AppNHp (biotin-EDA-AMPPNP); 8Br-cAMP; adenosine-3′,5′-bisphosphate; adenosine-2′,5′-bisphosphate; 2′-O-methyl-adenosine-3′,5′-bisphosphate (2′OMe-pAp); N⁶_{-methyl-ATP; AP4} (adenosine-5′-tetraphosphate); ara-ATP; and 3′-dATP.

Other suitable cytidine analogues may include 5-methyl-dCTP; 5-Aza-dCTP; 3TCMP; and 3TCTP.

Other suitable guanosine analogues may include cGMP; guanosine-3′,5′-bisphosphate (Gp); guanosine-2′,5′-bisphosphate; 8-oxo-GTP; 8-oxo-dGTP; m⁷GTP; and 2′ O-methyl-GTP (2′OMe-GTP).

Other suitable thymidine analogues may include AzTMP; AzTTP; d₄TMP; d₄TTP.

### Tus Polypeptides and Analogues and Derivatives Thereof

The amino acid sequence of an E. coli Tus polypeptide is shown below (SEQ ID NO: 5):

(SEQ ID NO: 5)

MARYDLVDRL NTTFRQMEQE LAAFAAHLEQ HKLLVARVFS
LPEVKKEDEH NPLNRIEVKQ HLGNDAQSQA LRHFRHLFIQ
QQSENRSSKA AVRLPGVLCY QVDNLSQAAL VSHIQHINKL
KTTFEHIVTV ESELPTAARF EWVHRHLPGL ITLNAYRTLT
VLHDPATLRF GWANKHIIKN LHRDEVLAQL EKSLKSPRSV
APWTREEWQR KLEREYQDIA ALPQNAKLKI KRPVKVQPIA
RVWYKGDQKQ VQHACPTPLI ALINRDNGAG VPDVGELLNY
DADNVQHRYK PQAQPLRLII PRLHLYVAD

The percentage identity to SEQ ID NO: 5 may be at least about 85%, more preferably at least about 90%, even more preferably at least about 95% and still more preferably at least about 99%.

For example, the Escherichia coli Tus protein is known in the art to be a monomeric 36-kDa protein that forms a simple 1:1 complex with a Ter site, as reviewed for example, by Hill, In: Escherichia coli and Salmonella: Cellular and Molecular Biology (Neidhardt F C, ed) Vol 2, pp 1602-1614, Am. Soc Microbiol, Washington D.C., USA and in Neylon et al., (2005), Microbiol. Mol. Biol. Rev. 69, 501-526.

“Homologues” of a Tus polypeptide may include any functionally-equivalent proteins to the Tus polypeptide of E. coli wherein said homologue is a naturally-occurring variant of said E. coli Tus having Ter binding activity.

Tus homologues may include those Ter family proteins, such as those of bacteria that are capable of specifically binding to one or more DNA replication terminus sites on the host and plasmid genome and block progress of the DNA replication fork (i.e., they function in “fork arrest”). “Ter family protein” refers to a DNA replication terminus site-binding protein (Ter protein) that is capable of specifically binding to a DNA replication terminus site on the host and plasmid genome such as, for example, to block progress of a DNA replication fork. The amino acid sequences of several such homologues are known in the art, e.g., from a bacterium selected from the group consisting of: Shigella flexneri (Jin et al., Nucleic Acids Res. 30 (20), 4432-4441 (2002); Salmonella enterica (McClelland et al., Nat. Genet. 36 (12), 1268-1274 (2004); Salmonella typhimarium (McClelland et al., Nature 413 (6858), 852-856 (2001); Klebsiella pneumoniae (Henderson et al., Mol. Genet. Genomics 265 (6), 941-953 (2001); Yersinia pestis (Song et al., DNA Res. 11 (3), 179-197 (2004); and Proteus vulgaris (Murata et al., J. Bacteriol. 184 (12), 3194-3202 (2002)).

“Analogues” of a Tus polypeptide may include any functionally-equivalent synthesized variants of the E. coli Tus polypeptide having Ter binding activity. Such analogues may, for example, comprise the amino acid sequence of a naturally-occurring E. coli Tus polypeptide with one or more non naturally-occurring amino acid substituents therein.

“Derivatives” of a Tus polypeptide may include any functionally-equivalent fragments of the E. coli Tus protein or a homologue or analogue thereof having Ter binding activity, and any fusion polypeptides comprising E. coli Tus polypeptide or a homologue or analogue thereof and another protein wherein said fusion polypeptide has Ter binding activity. Tus polypeptide derivatives may include a fusion polypeptide comprising Tus and a polypeptide to be displayed on a microwell or microarray surface or on the surface of a cell, phage, virus or in vitro.

As used herein, the term “Ter-binding activity” means the ability to bind to a naturally-occurring Ter site or to the double-stranded oligonucleotide of the present invention. Means for testing Ter-binding activity are described in the examples.

Tus derivatives may include fragments of a Ter family protein that retains the ability to bind to a Ter site notwithstanding that it may not necessarily be capable of specifically binding to one or more DNA replication terminus sites on the host and plasmid genome and/or block progress of the DNA replication fork or function in fork arrest.

The present invention encompasses conjugates of a Tus polypeptide having Ter binding activity, for example, linked to a protein of interest. Such a conjugate protein may be useful, for example, for displaying a protein of interest. Thus, the conjugate protein may be contacted to a solid surface coated with a TT-Lock nucleic acid of the invention for a time and under conditions for binding to occur, thereby displaying the protein of interest on the solid surface for, for example, use in an immunoassay.

The peptide, polypeptide or protein of interest may be conjugated to either end of the Tus protein or analogue, homologue or fragment with Ter binding activity or even conjugated to an internally region of the Tus polypeptide. The peptide, polypeptide or protein of interest and the Tus polypeptide may be capable of folding correctly and maintaining their distinct activities. Methods for conjugating two or more proteins are known in the art and described, for example, in Scopes (In: Protein Purification: Principles and Practice, Third Edition, Springer Verlag, 1994).

For example, two proteins may be linked by virtue of formation of a disulphide bond between a cysteine residue in each of the proteins. Should a protein comprise multiple cysteine residues, any of these cysteine residues may be replaced when they occur in parts of a polypeptide where their participation in a cross-linking reaction would likely interfere with biological activity. When a cysteine residue is replaced, it may be desirable to minimize resulting changes in polypeptide folding. Changes in polypeptide folding may be minimized when the replacement is chemically and sterically similar to cysteine, such as, for example, serine. Alternatively, or in addition, a cysteine residue may be introduced into a polypeptide for cross-linking purposes. The cysteine residue may be introduced at or near the amino- or carboxy-terminus of the peptide or polypeptide. Methods for the production of a polypeptide comprising a suitable cysteine residue, for example, a recombinant protein, will be apparent to the skilled artisan.

Following production of the polypeptides comprising suitable cysteine residues, cysteine residues may be oxidised using, for example, Cu(II)-(1,10-phenanthroline)₃ (CuPhe). The proteins may then be crosslinked using, for example, a dimaleimide (e.g., N,N′-o-phenylenedimaleimide (o-PDM), N,N′-p-phenylenedimaleimide (p-PDM) or bismaleimidohexane (BMH). Following quenching of the reaction (e.g., with DTT) cross-linked proteins may be isolated. Alternatively, photocross-linking of cysteine residues may be performed, for example, as described in Giron-Morzon et al., J. Biol. Chem., 279: 49338-49345, 2004.

In another embodiment, coupling of the two polypeptide constituents (or a polypeptide and another compound, for example, a small molecule) may be achieved using a coupling or conjugating agent, such as for example, a chemical cross-linking agent. Methods for the use of a chemical cross-linking reagent are known in that art and reviewed, for example, in Means et al. Bioconjugate Chemistry 1:2-12, 1990.

There are several intermolecular crosslinking reagents useful for the performance of the instant invention (see, for example, Means, G. B. and Feeney, R. E., Chemical Modification of Proteins, Holden-Day, 1974, pp. 39-43). Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which are relatively specific for sulfhydryl groups); and 1,5-difluoro-2,4-dinitrobenzene (which forms irreversible linkages with amino and tyrosine groups). Other crosslinking reagents useful for this purpose may include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

In this regard, a cross-linking reagent may be homobifunctional, that is, having two functional groups that undergo the same reaction. Homobifunctional crosslinking reagent may be bismaleimidohexane (BMH). BMH contains two maleimide functional groups, which may react specifically with sulfhydryl-containing compounds under mild conditions (pH 6.5-7.7). The two maleimide groups are connected by a hydrocarbon chain. Accordingly, BMH may be useful for irreversible attachment of a polypeptide to another molecule that contains one or more cysteine residues.

Alternatively, a crosslinking reagent may be heterobifunctional. A heterobifunctional crosslinking agent may have two different functional groups, for example, an amine-reactive group and a thiol-reactive group, that will cross-link two molecules having free amines and thiols, respectively. Such a heterobifunctional crosslinker may be useful for specific coupling methods for conjugating two chemical entities, thereby reducing the occurrences of unwanted side reactions such as homo-protein polymers. A variety of heterobifunctional crosslinkers are known in the art. Examples of heterobifunctional crosslinking agents may include succinimidyl 4-(N-maleimidomethyl)-cyclohexane-1-carboxylate (SMCC), N-succinimidyl (4-iodoacetyl) aminobenzoate (SIAB), 1-ethyl-3-(3-dimethylaminopropyl) carbodiimide hydrochloride (EDC); 4-succinimidyloxycarbonyl-a-methyl-a-(2-pyridyldithio)-toluene (SMPT), N-succinimidyl 3-(2-pyridyldithio) propionate (SPDP), succinimidyl 6-[3-(2-pyridyldithio) propionate] hexanoate (LC-SPDP)succinimidyl, m-maleimidobenzoyl-N-hydroxysuccinimide ester (MBS), and succinimide 4-(p-maleimidophenyl)butyrate (SMPB), an extended chain analog of MBS. The succinimidyl group of these crosslinkers may react with a primary amine, and the thiol-reactive maleimide may form a covalent bond with the thiol of a cysteine residue.

In addition, photoreactive crosslinkers, such as, for example and bis-[#-(4-azidosalicylamido)ethyl]disulfide (BASED) and N-succinimidyl-6-(4′-azido-2′-nitrophenyl-amino)hexanoate (SANPAH) may be useful for producing a protein conjugate.

As will be apparent from the foregoing, the present invention contemplates production of a protein conjugate by performing a process comprising contacting a Tus protein with Ter binding activity and a peptide, polypeptide or protein of interest with a compound capable of forming a bond between two proteins for a time and under conditions sufficient to form a bond thereby producing a conjugated protein.

The reagents described above are additionally useful for linking a protein to a non-proteinaceous compound, for example, a small molecule. In particular, the chemical cross-linking reagents described herein and known in the art may be useful for linking a Tus polypeptide with Ter binding activity to a compound of interest.

The present invention further encompasses the preparation and/or use of conjugates of a Tus protein having Ter binding activity, for example, linked to a protein of interest. Such a conjugate protein may be useful, for example, for displaying a protein of interest. For example, the conjugate protein may be contacted to a solid surface coated with a TT-Lock nucleic acid of the invention for a time and under conditions for binding to occur, thereby displaying the protein of interest on the solid surface for, for example, use in an immunoassay.

The peptide, polypeptide or protein of interest may be conjugated to either end of the Tus protein with Ter binding activity or conjugated to an internal region of the Tus protein. The peptide, polypeptide or protein of interest and the Tus protein may be capable of folding correctly and maintaining their distinct activities. Methods for conjugating two or more proteins are known in the art and described, for example, in Scopes (In: Protein Purification: Principles and Practice, Third Edition, Springer Verlag, 1994).

The present invention additionally contemplates the production of a fusion protein that comprises a Tus protein and a peptide, polypeptide or protein of interest. Methods for the production of a fusion protein are known in the art and described, for example, in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001).

The present invention further contemplates the production of a fusion protein that comprises a Tus protein and a peptide, polypeptide or protein of interest, together with a molecular tag, wherein said tag is suitable for immobilization of said fusion protein.

The tag may be selected from the group comprising hexa-histidine (His6), biotin ligase substrate sequences, FLAG, maltose binding protein or glutathione S transferase (GST). The tag may be His6 or biotin ligase substrate sequences. Other tags comprising a Tus polypeptide fused to a peptide, polypeptide or protein of interest are also contemplated by the present invention.

General methods for producing a recombinant fusion protein involve the production of nucleic acid that encodes said fusion protein. In this regard, the present invention provides a nucleic acid encoding a fusion protein comprising a Tus protein with Ter binding activity and a peptide, polypeptide or protein of interest. The fusion protein may be an in frame fusion.

As used herein, the term “in frame fusion” means that the nucleic acid encoding the Tus polypeptide with Ter binding activity and the nucleic acid encoding the peptide, polypeptide or protein of interest are in the same reading frame. Accordingly, transcription and translation of the nucleic acid results in expression of a single protein comprising both the Tus polypeptide with Ter binding activity and the peptide, polypeptide or protein of interest.

The nucleic acid encoding the constituent components of the fusion protein may be isolated using a known method, such as, for example, amplification (e.g., using PCR or splice overlap extension) or isolated from nucleic acid from an organism using one or more restriction enzymes or isolated from a library of nucleic acids or synthesized using a method known in the art and/or described herein. Methods for such isolation will be apparent to the ordinary skilled artisan.

For example, nucleic acid (e.g., genomic DNA or RNA that is then reverse transcribed to form cDNA) from a cell or organism comprising a protein of interest may be isolated using a method known in the art and cloned into a suitable vector. The vector may then be introduced into a suitable organism, such as, for example, a bacterial cell. Using a nucleic acid probe from the gene encoding the protein of interest, a cell comprising the nucleic acid of interest may be isolated using methods known in the art and described, for example, in Ausubel et al (In: Current Protocols in Molecular Biology. Wiley Interscience, ISBN 047 150338, 1987), Sambrook et al (In: Molecular Cloning: A Laboratory Manual, Cold Spring Harbor Laboratories, New York, Third Edition 2001).

Alternatively, nucleic acid encoding a protein of interest may be isolated using polymerase chain reaction (PCR). Methods of PCR are known in the art and described, for example, in Dieffenbach (ed) and Dveksler (ed) (In: PCR Primer: A Laboratory Manual, Cold Spring Harbour Laboratories, NY, 1995). Generally, for PCR two non-complementary nucleic acid primer molecules comprising at least about 20 nucleotides in length, and more preferably at least 25 nucleotides in length may be hybridized to different strands of a nucleic acid template molecule, and specific nucleic acid molecule copies of the template may be amplified enzymatically. The primers may hybridize to nucleic acid adjacent to a gene or coding region encoding the protein of interest, thereby facilitating amplification of the nucleic acid that encodes the subunit. Following amplification, the amplified nucleic acid may be isolated using methods known in the art.

Other methods for the production of an oligonucleotide of the invention will be apparent to the skilled artisan and are encompassed by the present invention.

Following isolation of each of the components of the fusion protein, a fusion protein encoding nucleic acid may be produced, for example, by ligating the two coding regions together in frame such that a single protein is produced, e.g., using a DNA ligase. Alternatively, an amplification reaction may be performed using one or more primers that are capable of hybridizing to both components and thereby produce a single nucleic acid molecule.

The nucleic acid may additionally include regions that encode, for example, a linker or spacer region, a detectable marker and/or a further fusion protein. For example, a nucleic acid encoding a linker or spacer region may be included between the Tus protein with Ter binding activity and the peptide, polypeptide or protein to facilitate correct folding of each of the constituent components of the fusion protein. The linker may have a high freedom degree for linking of two proteins, for example a linker comprising glycine and/or serine residues. Suitable linkers are described, for example, in Robinson and Sauer Proc. Natl. Acad. Sci. 95: 5929-5934, 1998 or Crasto and Fang, Protein Engineering, 13: 309-312, 2000.

Following isolation of the nucleic acid encoding the fusion protein, an expression construct that comprises nucleic acid encoding the fusion protein of the invention may be produced. As used herein, the term “expression construct” shall be taken to mean a nucleic acid molecule that has the ability confer expression of a nucleic acid fragment to which it is operably connected, in a cell or in a cell free expression system. Within the context of the present invention, it is to be understood that an expression vector that comprises a promoter as defined herein may be a plasmid, bacteriophage, phagemid, cosmid, virus sub-genomic or genomic fragment, or other nucleic acid capable of maintaining and or replicating heterologous DNA in an expressible format should it be introduced into a cell. Many expression vectors are commercially available for expression in a variety of cells. Selection of appropriate vectors is within the knowledge of those having skill in the art. The present invention contemplates an expression vector comprising a nucleic acid encoding a fusion protein of the invention.

As will be apparent from the foregoing, an expression construct useful for the production of a fusion protein of the invention may comprise a promoter. The nucleic acid comprising the promoter sequence may be isolated using a technique known in the art, such as for example PCR or restriction digestion. Alternatively, the nucleic acid comprising the promoter sequence may be synthetic, for example, an oligonucleotide.

The term “promoter” is to be taken in its broadest context and includes the transcriptional regulatory sequences of a genomic gene, including the TATA box or initiator element, which may be required for accurate transcription initiation, with or without additional regulatory elements (ie. upstream activating sequences, transcription factor binding sites, enhancers and silencers) which alter gene expression in response to developmental and/or external stimuli, or in a tissue specific manner. In the present context, the term “promoter” is also used to describe a recombinant, synthetic or fusion molecule, or derivative which confers, activates or enhances the expression of a nucleic acid molecule to which it is operably linked, and which encodes the peptide or protein. Preferred promoters may contain additional copies of one or more specific regulatory elements to further enhance expression and/or alter the spatial expression and/or temporal expression of said nucleic acid molecule.

Placing a nucleic acid molecule under the regulatory control of a promoter sequence may involve positioning said molecule such that expression is controlled by the promoter sequence. Promoters are generally positioned 5′ (upstream) to the coding sequence that they control. To construct heterologous promoter/structural gene combinations, the promoter may be positioned at a distance from the gene transcription start site that is approximately the same as the distance between that promoter and the gene it controls in its natural setting, that is, the gene from which the promoter is derived. As is known in the art, some variation in this distance may be accommodated without loss of promoter function. Similarly, the preferred positioning of a regulatory sequence element with respect to a heterologous gene to be placed under its control may be defined by the positioning of the element in its natural setting, that is, the gene from which it is derived. As is known in the art, some variation in this distance can also occur.

Should it be preferred that the fusion protein be expressed in vitro, a suitable promoter may include, but is not limited to, the T3 or T7 bacteriophage promoters (Hanes and Plückthun Proc. Natl. Acad. Sci. USA, 94 4937-4942 1997).

Typical expression vectors for in vitro expression or cell-free expression have been described and include, but are not limited to the TNT T7 and TNT T3 systems (Promega), the pEXP1-DEST and pEXP2-DEST vectors (Invitrogen).

Typical promoters suitable for expression in bacterial cells include, but are not limited to, the lacZ promoter, the lpp promoter, temperature-sensitive λL or λR promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are known in the art and are described for example, in Ausubel et al (In: Current Protocols in Molecular Biology. Wiley Interscience, ISBN 047 150338, 1987), U.S. Pat. No. 5,763,239 (Diversa Corporation) and Sambrook et al (In: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have been described, and include, for example, PKC30 (Shimatake and Rosenberg, Nature 292: 128, 1981); pKK173-3 (Amann and Brosius, Gene 40: 183, 1985), pET-3 (Studier and Moffat, J. Mol. Biol. 189: 113, 1986); the pCR vector suite (Invitrogen), pGEM-T Easy vectors (Promega), the pBAD/TOPO (Invitrogen), the pFLEX series of expression vectors (Pfizer nc., CT, USA), the pQE series of expression vectors (QIAGEN, CA, USA), or the pL series of expression vectors (Invitrogen), amongst others.

Typical promoters suitable for expression in a mammalian cell, mammalian tissue or intact mammal include, for example a promoter selected from the group consisting of, a retroviral LTR element, a SV40 early promoter, a SV40 late promoter, a cytomegalovirus (CMV) promoter, a CMV IE (cytomegalovirus immediate early) promoter, an EF_{1α} promoter (from human elongation factor la), an EM7 promoter or an UbC promoter (from human ubiquitin C).

Expression vectors that contain suitable promoter sequences for expression in mammalian cells or mammals include, but are not limited to, the pcDNA vector suite supplied by Invitrogen, the pCI vector suite (Promega), the pCMV vector suite (Clontech), the pM vector (Clontech), the pSI vector (Promega) or the VP16 vector (Clontech).

As will be apparent from the foregoing, the present invention provides a method for producing an expression construct encoding a fusion protein of the invention comprising placing a nucleic acid encoding the fusion protein in operable connection with a promoter.

Furthermore, the present invention provides a vector comprising a nucleic acid encoding a fusion protein comprising a Tus polypeptide or an analogue, homologue or fragment thereof and a peptide, polypeptide or protein of interest.

Following production of a suitable expression construct, a recombinant fusion protein may be produced. This may involve introducing the expression construct into a cell for expression of the recombinant protein. Methods for introducing an expression construct into a cell for expression are known to those skilled in the art and are described for example, in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001). The method chosen to introduce the gene construct depends upon the cell type in which the gene construct is to be expressed. Means for introducing recombinant DNA into cells include, but are not limited to electroporation, chemical transformation into cells previously treated to allow for said transformation, PEG mediated transformation, microinjection, transfection mediated by DEAE-dextran, transfection mediated by calcium phosphate, transfection mediated by liposomes such as by using Lipofectamine (Invitrogen) and/or cellfectin (Invitrogen), transduction by Adenoviuses, Herpesviruses, is Togaviruses or Retroviruses and microparticle bombardment such as by using DNA-coated tungsten or gold particles (Agacetus Inc., WI, USA).

Following transformation or transfection, cells may be incubated for a time and under conditions sufficient for expression of the fusion protein. If a purified fusion protein is desired, the protein may then be isolated by a method known in the art, such as, for example, by affinity purification. Methods for the isolation of a protein are known in the art and/or described in Scopes (In: Protein Purification: Principles and Practice, Third Edition, Springer Verlag, 1994).

In an alternative embodiment, the fusion protein may be produced in vitro, using an in vitro expression system as described above. Such a system may be used to translate a previously produced RNA molecule, for example, using a rabbit reticulocyte lysate (available from Promega Corporation) or to transcribe and/or translate a nucleic acid construct (e.g., a DNA construct), for example, using an E. coli extract (also available from Promega Corporation). Various kits for in vitro transcription/translation are commercially available. Following in vitro production, the fusion protein may be isolated or purified using, for example, affinity purification.

### Surface Plasmon Resonance Chips

The inventors have also developed a real-time exonuclease assay based on use of a surface plasmon resonance (SPR; Biacore) sensor. The main use of SPR is to study binding interactions. The most common surfaces for the immobilization of oligonucleotides are streptavidin (SA) chips. Unfortunately the binding between SA and a biotinylated oligonucleotide is essentially irreversible, making the use of such a surface for the study of exonuclease kinetics very costly—the chip surface can be used only once. Therefore, it was important to design an oligonucleotide-binding surface that could be regenerated for subsequent reactions. The inventors thus employed the TT-Lock technology to produce a new Biacore chip surface that can bind oligonucleotides in a reversible fashion.

Accordingly, the present invention provides for a chip, wherein said chip comprises the double-stranded oligonucleotides or the conjugates as described herein.

### Display Formats

In one embodiment, the double-stranded oligonucleotides of the present invention may be used for in vitro display, such as ribosome display, ribosome inactivation, covalent display or mRNA display.

The present invention accordingly provides methods for presenting or displaying a molecule such as a polypeptide, nucleic acid, antibody or small molecule on a surface, said method comprising contacting a conjugate comprising the double-stranded oligonucleotide as described herein covalently bound to the molecule with a Tus polypeptide having TerB binding activity bound to the surface for a time and under conditions sufficient to form a DNA/protein complex wherein the molecule is displayed on the surface.

Optionally, the method further comprises cross-linking the double-stranded nucleic acid moiety of the conjugate to the Tus polypeptide or a homologue, analogue or derivative thereof, for example, using formaldehyde.

These embodiments may be particularly suitable for presenting or displaying nucleic acid, in which case the conjugate comprises the double-stranded oligonucleotides bound to DNA or RNA. However, it is to be understood that this embodiment of the invention is also useful for presenting or displaying any other molecule capable of being conjugated to nucleic acid, particularly to single-stranded or double-stranded DNA. For example, the oligonucleotides of the present invention may be conjugated to a protein for use in a forward or reverse hybrid assay (e.g., to identify a ligand of a protein or to identify a receptor agonist or antagonist) or immunoassay (e.g., ELISA), or to an antibody for use such as for use in epitope mapping or immunoassay, or to a small molecule for use in screening applications (e.g., to screen for an agonist or antagonist of a receptor protein). Other applications are not to be excluded.

The surface may be any surface suitable for a nucleic acid hybridization (RNA/DNA, RNA/RNA or DNA/DNA hybridization) or for analysing the interaction of a nucleic acid, protein, antibody or small molecule with nucleic acid. As will be known to the skilled artisan, this may include the surface of a microwell or a glass, nylon or composite material suitable for producing a microarray, a polymeric pin, or chromatographic material e.g., agarose, Sepharose, cellulose, polyacrylamide, etc.

The surface may be prepared or provided in a ready-to-use format and the present invention encompasses the preparation of the surface for use. Accordingly, in one embodiment, the method further comprises the first step of contacting the surface with the Tus polypeptide, homologue, analogue or derivative for a time and under conditions sufficient for said polypeptide to bind to said surface. The binding may be covalent or non-covalent, for example, electrostatic or van der Waals interaction.

Subject to the proviso that the double-stranded oligonucleotide has not been cross-linked to a Tus polypeptide, the surface, once prepared, is readily reusable. Accordingly, in another preferred embodiment, the method further comprises disrupting the DNA/protein complex and contacting a conjugate comprising a double-stranded oligonucleotide as described herein covalently bound to a molecule (e.g., a second molecule different to the first molecule) with the Tus polypeptide having TerB binding activity for a time and under conditions sufficient to form a DNA/protein complex wherein the molecule is displayed on the surface by virtue of said interaction.

The invention also encompasses such display formats in the reverse or opposite format wherein the oligonucleotides of the invention are bound to a surface and a conjugate comprising a Tus polypeptide is bound reversibly thereto. Such a reverse format may be suitable for presenting or displaying any polypeptide or peptide that can be produced as a fusion polypeptide with Tus or chemically added thereto, for example, in preparation for a forward or reverse hybrid assay (for example, to identify a ligand of a protein or to identify a receptor agonist or antagonist) or immunoassay (e.g., ELISA). However, it is to be understood that any other molecule capable of being conjugated to protein may be displayed in accordance with this embodiment. For example, a Tus protein may be conjugated to a nucleic acid for use in a hybridization assay. Alternatively, a Tus protein may be conjugated to an antibody for use in epitope mapping or an immunoassay, or to a small molecule for use in screening applications (for example, to screen for an agonist or antagonist of a receptor protein). Other applications are not to be excluded.

Accordingly, a further embodiment of the present invention provides a method for presenting or displaying a molecule such as a polypeptide, nucleic acid, antibody or small molecule on a surface, said method comprising contacting a conjugate comprising a Tus polypeptide having TerB binding activity covalently bound to the molecule to a double-stranded oligonucleotide as described herein bound to the surface for a time and under conditions sufficient to form a DNA/protein complex, wherein the molecule is displayed on the surface by virtue of said interaction.

The surface may be the surface of a microwell or a glass, nylon or composite material suitable for producing a microarray, a polymeric pin, or chromatographic material, for example, agarose, Sepharose, cellulose or polyacrylamide. The oligonucleotide may be bound to the surface by any means, e.g., by cross-linking or other covalent attachment or by electrostatic interaction with the surface, the only requirement being that it is capable of binding to a Tus polypeptide when bound to the surface.

Optionally, the method further comprises cross-linking the double-stranded oligonucleotide moiety of the conjugate to the Tus polypeptide, for example, by using formaldehyde.

The surface may be prepared or provided in a ready-to-use format and the present invention therefore encompasses the preparation of the surface for use. In one preferred embodiment, the method further comprises the first step of contacting the surface with the double-stranded oligonucleotides as described herein for a time and under conditions sufficient for said oligonucleotide to bind to said surface.

Subject to the proviso that the Tus polypeptide conjugate has not been cross-linked to the double-stranded oligonucleotide, the surface may be reused. Accordingly, in a preferred embodiment, the method further comprises disrupting the DNA/protein complex and contacting a conjugate comprising a Tus polypeptide having TerB binding activity covalently bound to a molecule (for example, a second molecule different to the first molecule) with the oligonucleotide for a time and under conditions sufficient to form a DNA/protein complex, wherein the molecule is displayed on the surface by virtue of said interaction.

In other embodiments, the double-stranded oligonucleotides of the present invention may be used in a method of displaying mRNA or a polypeptide molecule or a conjugate comprising mRNA and a polypeptide encoded by it, wherein the mRNA or polypeptide molecule is displayed as part of a conjugate with the nucleic acid, or alternatively, as a capture reagent to assist in recovery of an mRNA or a polypeptide displayed as part of a conjugate with a Tus protein. The mRNA or polypeptide may be displayed on the surface of a ribosome,

For example, the present invention provides a method of presenting or displaying a molecule comprising incubating a conjugate comprising a double-stranded oligonucleotide as described herein covalently bound to mRNA for a time and under conditions sufficient for partial or complete translation of the mRNA to occur, thereby producing a complex comprising the conjugate, a nascent polypeptide encoded by the mRNA and optionally a ribosome.

It is within the scope of the present invention for the conjugate to be covalently linked to puromycin for terminating translation. Alternatively, or in addition, the conjugate may be linked to a psoralen moiety to facilitate cross-linking of the mRNA to the nascent polypeptide.

As used herein, the term “partial or complete translation” shall be taken to mean that sufficient translation of mRNA occurs to produce a nascent polypeptide encoded by the mRNA to be detected e.g., by virtue of its activity or binding to a ligand (for example, a small molecule, antibody, protein binding partner, DNA recognition site, receptor, etc). As will be known to the skilled artisan, translation of a full-length polypeptide is not essential for such detection, and for most applications a polypeptide of at least 5-10 amino acids in length is generally sufficient.

The term “conditions sufficient for partial or complete translation” means incubation of the mRNA conjugate in the presence of sufficient components of a suitable in vitro translation system e.g., wheat germ, reticulocyte lysate, or S-30 translation system. Commercially-available translation systems can be used. The methods disclosed herein are not limited to presentation or display involving eukaryotic mRNAs, as prokaryotic mRNAs are also contemplated. Accordingly, the in vitro translation system may be suitable for the translation of eukaryotic mRNA, on eukaryotic 80S ribosomes, or alternatively for the translation of prokaryotic mRNAs on 70S ribosomes.

The term “nascent polypeptide” means a growing polypeptide chain produced by translation. In the present context, the term “nascent polypeptide” may be, but is not necessarily limited to, that part of a growing polypeptide chain exiting the ribosome.

Translation may be inactivated or stalled by contacting the incubating conjugate with a Tus polypeptide for a time and under conditions sufficient for the double-stranded oligonucleotide to bind to the Tus polypeptide, thereby stalling translation. Optionally, the double-stranded oligonucleotide moiety of the conjugate in the stalled translation mixture may be cross-linked to Tus polypeptide, for example, using formaldehyde, to stabilize the complex.

Alternatively, or in addition, the complex between the mRNA conjugate, a nascent polypeptide encoded by the mRNA and optionally a ribosome may be stabilized by addition of a reagent such as, for example, magnesium acetate or chloramphenicol.

In an embodiment, the conjugate may further comprise one or more nucleotide sequences selected from the group consisting of:

(i) a sequence capable of targeting the mRNA to a ribosome (e.g., a ribosome binding sequence);

(ii) a sequence encoding an amino acid sequence capable of stabilizing the nascent polypeptide within the ribosomal tunnel (e.g., a sequence encoding amino acids 211-299 of gene III of phage M13 mp19);

(iii) a spacer sequence (e.g., encoding an amino acid sequence that is rich in glycine and/or serine and/or proline);

(iv) a sequence encoding a polypeptide that interacts with a recognition site within it (e.g., E. coli bacteriophage P2A-encoding sequence or a homologue thereof in phage 186 or phage HP1 or phage PSP3);

(v) a sequence encoding a toxin peptide (e.g., a ricin A-encoding sequence); and

(vi) a combination of one or more of (i) to (v).

The method may further comprise recovering the complex produced according to the preceding embodiments using an affinity tag or ligand for one or more components of the complex, that is, the oligonucleotide, the nascent polypeptide, the mRNA, one or more additional sequences included in the conjugate, or the ribosome. For example, the complex may be recovered by contacting the complex with an antibody against the nascent polypeptide for a time and under conditions sufficient for an antigen-antibody complex to form. Alternatively, or in addition, the complex may be contacted with a Tus polypeptide for a time and under conditions sufficient for the double-stranded oligonucleotide to bind to the Tus polypeptide, followed by recovery of the complex.

The double-stranded oligonucleotides of the present invention may also be used to recover a complex formed during ribosome display, ribosome inactivation display, mRNA display, covalent display, phage display, retroviral display, bacterial display, yeast display, mammalian cell display or other presentation or display format, wherein the displayed integer is a fusion with a Tus polypeptide or mRNA encoding said Tus polypeptide.

Accordingly, the present invention provides a method for presenting or displaying a molecule, wherein said method comprises:

(i) incubating an mRNA conjugate, wherein said mRNA conjugate comprises mRNA encoding a Tus polypeptide having TerB binding activity fused to mRNA encoding a second polypeptide, for a time and under conditions sufficient for translation of the Tus polypeptide to be produced, and partial or complete translation of the mRNA encoding the second polypeptide to occur, thereby producing a complex comprising the conjugate, a nascent Tus-polypeptide fusion protein encoded by the conjugate and optionally a ribosome;

(ii) incubating the complex with a double-stranded oligonucleotide as described herein for a time and under conditions sufficient to bind to said Tus polypeptide; and

(iii) recovering the complex.

The Tus polypeptide may be fused to mRNA encoding a second polypeptide in the same reading frame.

Optionally, the double-stranded nucleic acid may be cross-linked to the Tus polypeptide, for example, using formaldehyde, to stabilize the complex. It is also within the scope of the present invention for the mRNA conjugate to be linked to a psoralen moiety to facilitate cross-linking of the mRNA to the nascent polypeptide.

Alternatively, or in addition, the mRNA conjugate may be covalently linked to puromycin for terminating translation.

In the cell-based and in vitro display formats described herein, the complex, including any recovered complex, may be subjected to reverse transcription (RT) to produce cDNA and/or be amplified by any means known in the art such as polymerase chain reaction (e.g., PCR or RT-PCR) to thereby produce DNA copies of an mRNA of interest (i.e., the mRNA conjugated to the double-stranded nucleic acid or to mRNA encoding the Tus moiety, as the case may be). Optionally, one or more mutations may be incorporated during the amplification process to create or enhance sequence diversity in the pool of DNA molecules produced.

The present invention also encompasses the additional first step of providing a conjugate comprising mRNA encoding a protein of interest fused, which may be in-frame, to mRNA encoding a Tus polypeptide, or alternatively, fused to a double-stranded oligonucleotide as described herein.

In the present context, the term “providing a conjugate” includes providing the double-stranded oligonucleotide or mRNA encoding a Tus polypeptide, and/or providing mRNA encoding a protein of interest and/or conjugating the double-stranded oligonucleotide to the mRNA and/or conjugating the mRNA encoding Tus to mRNA encoding the protein of interest.

The present invention also encompasses the provision of mRNA of interest and optionally, variant sequences thereto, by a process comprising transcribing DNA into mRNA in the presence of a DNA-dependent RNA polyerase. As will be known to the skilled artisan, the use of an error-prone RNA polymerase such as Q-replicase permits the introduction of errors into the mRNA sequence thereby producing a large number of related sequences to the sequence of interest for subsequent screening to determine those having modified affinity (i.e, directed evolution).

The present invention also provides a process for presenting or displaying a molecule, wherein said process comprises:

(i) providing DNA encoding a fusion protein comprising a Tus polypeptide having TerB binding activity fused to a polypeptide of interest;

(ii) transcribing the DNA in the presence of an RNA polymerase to produce an mRNA conjugate comprising mRNA encoding a Tus polypeptide fused to mRNA encoding the polypeptide of interest;

(iii) incubating the mRNA conjugate for a time and under conditions sufficient for translation of a Tus polypeptide to be produced, and partial or complete translation of the mRNA encoding the polypeptide of interest to occur, thereby producing a complex comprising the conjugate, a nascent Tus-fusion protein encoded by the conjugate and optionally a ribosome;

(iv) incubating the complex with a double-stranded oligonucleotide as described herein for a time and under conditions sufficient to bind to said Tus polypeptide; and

(v) recovering the complex.

The RNA polymerase may be an error-prone RNA polymerase, for example, Qβ-replicase, the use of which introduces nucleotide substitutions into the nucleotide sequence of the transcript. By fine-tuning the mutation rate, for example, to the rate of about 1-10 mutations per molecule being transcribed or greater, a highly diverse library of related mRNA transcripts may be produced, which may be selected at the recovery stage on the basis of the ability of such transcripts to bind to a ligand at a particular affinity as well as maintaining their ability to bind to the double-stranded oligonucleotides of the present invention. For example, mutations can be introduced into mRNA encoding a variable chain of an antibody that binds to a polypeptide, thereby producing a library of antibody variable chains, from which are selected those mRNAs encoding variable chains having enhanced binding activity to the polypeptide.

### Kits

The present invention also provides kits for producing the double-stranded nucleic acid molecule as described above, and for presenting or displaying a molecule, wherein the kits facilitate the employment of the methods and processes of the invention. Typically, kits for carrying out a method of the invention contain all the necessary reagents to carry out the method. Typically, the kits of the invention will comprise one or more containers, containing for example, wash reagents, and/or other reagents capable of releasing a bound component from a polypeptide or fragment thereof.

In the context of the present invention, a compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept a test sample, a container which contains the polymers used in the assay and containers which contain wash reagents (such as phosphate buffered saline, Tris-buffers, and like).

Typically, a kit of the present invention will also include instructions for using the kit components to conduct the appropriate methods.

Methods and kits of the present invention find application in any circumstance in which it is desirable to purify any component from any mixture.

The present invention provides kits comprising a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce the double-stranded nucleic acid molecule as described above.

The oligonucleotide or an analogue or derivative thereof may be provided in solution or as a solid e.g., a precipitate, or bound directly or indirectly to a solid matrix (e.g., a microwell, glass, nylon or composite material suitable for microassay, including a BIAcore chip, protein display chip, glass bead, microdot or quantum dot), a proteinaceous molecule, nucleic acid or small molecule. For example, the double-stranded oligonucleotide of the present invention can be bound covalently or cross-linked to a nucleic acid (e.g., mRNA), polypeptide (e.g., puromycin) or small molecule (e.g., psoralen, pyrido[3,4-c]psoralen or 7-methylpyrido[3,4-c]-psoralen). Alternatively, or in addition, the double-stranded oligonucleotide of the present invention can be bound non-covalently to a Tus protein or a homologue, analogue or derivative thereof.

The present invention further provides kits for presenting or displaying a first molecule, wherein said first molecule comprises a double-stranded nucleic acid molecule as described above, in a form suitable for conjugating to:

(a) a second molecule, wherein said second molecule comprises a nucleic acid, polypeptide or small molecule; and

(b) an integer selected from the group consisting of:
(i) a Tus polypeptide or a homologue, analogue or derivative thereof in a form suitable for conjugating to another molecule, wherein said double-stranded nucleic acid molecule and said Tus polypeptide interact in use to present or display another molecule conjugated to said double-stranded nucleic acid molecule or said polypeptide; and
(ii) mRNA encoding a Tus polypeptide or a homologue, analogue or derivative thereof in a form suitable for conjugating to mRNA encoding another polypeptide.

### Other Variations and Modifications

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts which are incorporated herein by reference:
1. Sambrook, Fritsch & Maniatis, whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakalibara, D., Teichman, J., Lien, E. Land Fenichel, R. L. (1976). Biochem. Biophys. Res. Commun. 73 336-342
11. Merrifield, R. B. (1963). J. Am. Chem. Soc. 85, 2149-2154.
12. Barany, G. and Merrifield, R. B. (1979) in The Peptides (Gross, E. and Meienhofer, J. eds.), vol. 2, pp. 1-284, Academic Press, New York.
13. Wünsch, E., ed. (1974) Synthese von Peptiden in Houben-Weyls Metoden der Organischen Chemie (Müller, E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Peptide Protein Res. 25, 449-474.
17. Handbook of Experimental Immunology, Vols. I-IV (D. M. Weir and C. C. Blackwell, eds., 1986, Blackwell Scientific Publications).
18. McPherson et al., In: PCR A Practical Approach., IRL Press, Oxford University Press, Oxford, United Kingdom, 1991.
19. Stears et al. (2003) “Trends in microarray analysis” Nature Medicine 9, 140-145.
20. He et al., “Ribosome Display: Cell-free protein display technology” Briefings in Functional genomics and proteomics 1, 204-212, 2002.

The present invention is further described with reference to the following non-limiting examples.

### Example 1

### Kinetic and Thermodynamic Properties of Double-Stranded Nucleic Acids

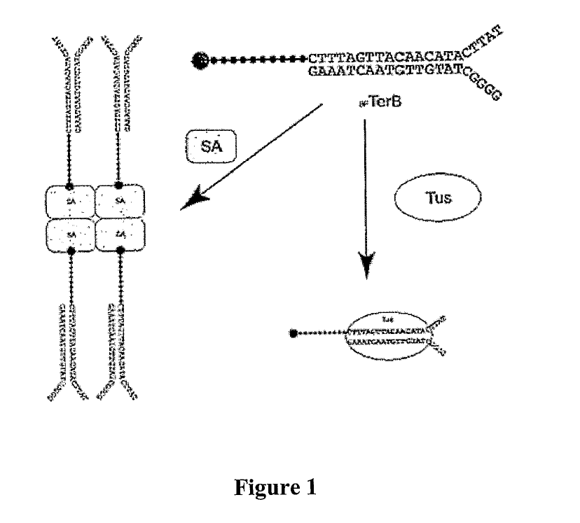

To compare the kinetic and thermodynamic binding parameters of Tus polypeptides and streptavidin (SA) with their respective ligands, a biotinylated forked TerB site (BFTerB) and derivatives thereof were used as a universal ligand (FIG. 1). BFTerB can bind to both SA through the 5′-biotin moiety or to Tus through its DNA sequence (FIG. 1). Since the ligand is the same for both interacting partners, it was expected that artifactual effects frequently seen in BIAcore studies, such as mass transfer effects, would be about the same in both kinetic studies.

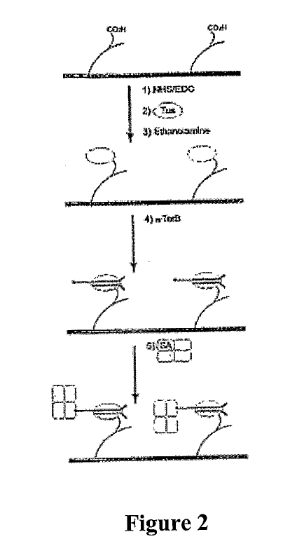

Surfaces of the commonly used CM5 chip for the BIAcore 2000 instrument (Biacore) were modified to display similar amounts of both SA and Tus proteins in two separate flow cells. Then, a solution of BFTerB was made to flow over the surfaces, followed by SA, and the instrument responses (R) were recorded (FIG. 2). To confirm the utility of the Tus-BFTerB surface for other applications, its stability and regenerability was checked under various conditions.

### 1.1 Materials and Methods

### 1.1.1 Reagents and Buffers

The running and dialysis buffer, HBS-PD, was made of HBS-P (Biacore) supplemented with 1/1000 volume of 1 M DTT; the hybridization buffer (HyB) contained 10 mM Tris-HCl, pH 8 and 0.5 M NaCl; regeneration buffers were either 1 M MgCl₂ (RB 1) or 50 mM NaOH in 1 M NaCl (RB2); dissociation buffers (DB) were 10 mM HEPES (pH 7.4), 1 mM DTT, with varying NaCl concentrations between 0 mM and 300 mM. A stock solution made of 5 mg/ml SA (lyophilized, Sigma) was prepared in HBS-PD. The Tus protein was prepared as described by Neylon et al., Biochemistry, 39, 11989-11999 (2000) and was buffer exchanged five times against HBS-PD with an Amicon Ultra-4 centrifugal ultrafiltration device (MWCO 10000 Da) and concentrated to a ca. 6 mg/ml stock solution. Oligonucleotides were custom made by GeneWorks, Australia. Oligo654 had been derivatized at the 5′ end with biotin followed by a 10-residue abasic deoxyribose phosphate spacer).

1.1.2 BIAcore studies

The Biacore 2000 was set up at a working temperature of 20° C. and a constant flow rate of 5 μl/min. Prior to derivatization of the surface of a CM5 sensor chip (Biacore), the Tus and SA stock solutions were diluted to final concentrations of 2.77 U in 10 mM HEPES (pH 7.4) and 6.66 μM in 10 mM sodium acetate (pH 4.6), respectively. The flow cells of the CM5 chip were activated with a freshly prepared solution of NHS and EDC during 7 min as recommended by Biacore, followed by a 7-min injection of Tus or two sequential 7-min injections of SA. Both surfaces were neutralized with 1 M ethanolamine (pH 8.5) during 7 min.

A stock solution (10 μM) of the universal ligand BFTerB was prepared by mixing 2 μl of oligo838 (100 μM) with 2 μl of oligo654 (100 μM) and 16 μl of HyB, followed by treatment for 5 min at 65° C. and slow cooling to 20° C. It was then diluted to 250 nM in HBS-PD and caused to flow over the SA and Tus flow cells; the recorded instrument responses (RU) were compared. To confirm that BFTerB was able to fully display the 5′ biotin moiety, further 2-4 min injections of SA (1 or 2 μM) were carried out and used as an indirect quantification method. The surfaces were regenerated with either RB1 or RB2.

### 1.2 Results and Discussion

### 1.2.1 Comparison of Maximum Binding Activity of Immobilized Proteins

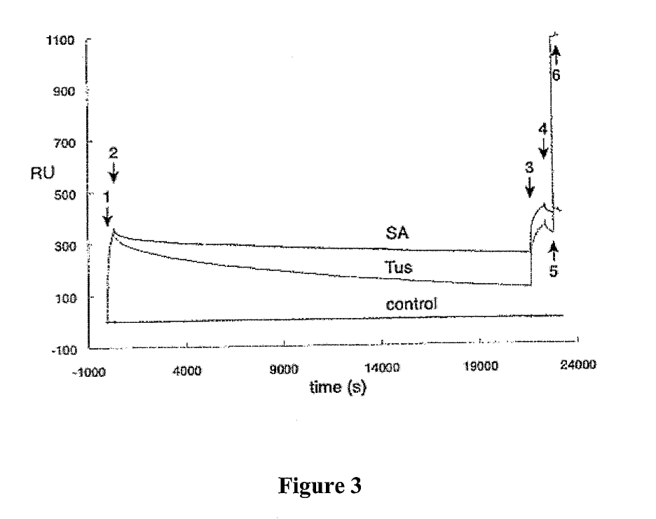

Tus (36 kDa) and SA (15 kDa monomer) were covalently immobilized on a standard CM5 chip surface to yield increases of RU of 6800 (189 molar units) and 5640 (376 molar units), respectively. The immobilization time for SA was twice as long as for Tus, using protein concentrations of 1.6 μM (tetrameric SA) and 2.8 μM (Tus). This suggest that the coupling rates are similar for the two proteins, and about twice as many molecules of SA than Tus were immobilized in two subsequent injections. It was expected that SA would bind about twice as much BFTerB in the same time interval if the kinetic and thermodynamic parameters were equivalent. Using a 250 nM solution of BFTerB, we achieved similar levels of binding (Rₘₐₓ) on both surfaces with a slightly faster apparent on-rate in the case of Tus. Thus, the concentration of active binding sites on the two surfaces are comparable, even though twice as many molecules of SA (as monomer) were immobilized. At least 15% of immobilized Tus was still capable of binding BFTerB, notwithstanding that the forked TerB may be threaded into the Tus molecule on binding. In the running buffer containing 150 mM NaCl at 20° C., the dissociation rates of BFTerB from the Tus and SA surfaces were not much different; BFTerB dissociated from Tus about twice as fast as from SA (FIG. 3).

### 1.2.2 Regeneration

BFTerB may be stripped from the Tus and SA surfaces with 50 mM NaOH in 1 M NaCl (RB2). The Tus surface, like the SA surface, was able to cope with numerous one-minute injections of RB2 without significant loss of BFTerB-binding capacity, although some undesirable artifacts occurred from time to time (e.g., increase in baseline).

It was subsequently found that the Tus surface could be successfully and reproducibly regenerated under much milder conditions, using two sequential 4-minutes injections of 1 M MgCl₂ (RBI). To check if this regeneration method was sufficient to deal with more complex situations, the displayed biotin moiety of Tus surface-bound BFTerB was saturated with additional SA. In this case the surface was completely protected from binding further BFTerB, and was probably an interconnected network of SA tetramers. This surface was extremely stable; there was absolutely no sign of its degradation over several hours. After regeneration by the traditional method (RB2), no SA could be bound, demonstrating that BFTerB had been completely removed. However, the baseline did not return to its normal value. Only when RB 1 was used did the baseline return cleanly to normal, and an equivalent RU of BFTerB and subsequently SA could again be bound.

### 1.2.3 Time Stability

The Tus surface was challenged approximately 70 times with different concentrations of oligonucleotides, SA and different regeneration conditions over a period of 4 days at 20° C. Some extreme conditions were tested for regeneration, e.g., nine 1-min injections of RB2, four long injections of 10 mM HEPES containing 1 M NaCl, an injection of 5 M NaCl, an injection of deionized water, and 13 long injections of RBI. This resulted in a 25% decrease of BFTerB (250 nM) binding capacity from 350 to 260 RU. It is important to note that no significant loss of binding capacity occurred after RB 1 was adopted as the standard regeneration condition.

### 1.2.4 Stability to NaCl

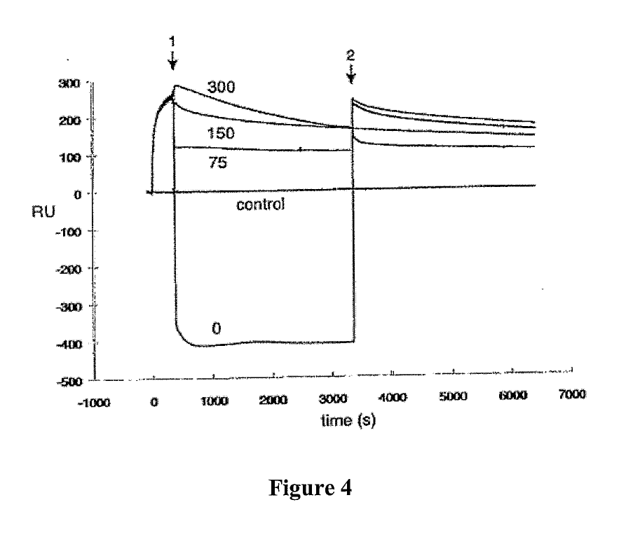

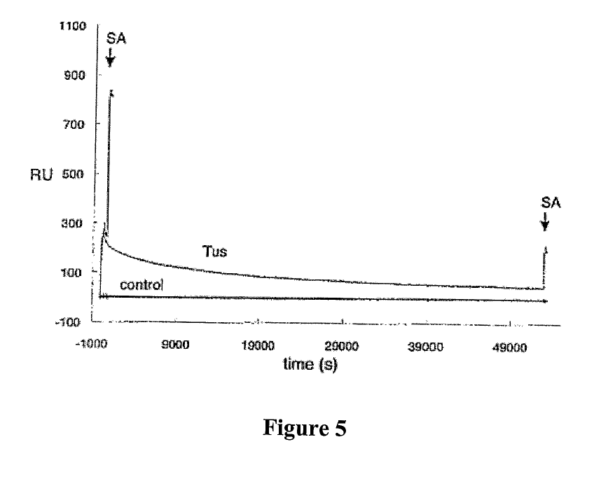

The interaction of Tus with BFTerB is influenced by ionic strength. To establish the effect of ionic strength on the stability of the complex of BFTerB with immobilized Tus, dissociation buffer (DB) solutions with NaCl concentrations ranging from 0-300 mM were injected during 50 min, immediately after 5 min of binding of BFTerB (250 nM). With 75 mM NaCl, no loss of BFTerB was observed, so the half life is at least about one day. At 150 mM NaCl, the half life was estimated to be about 4 h, and at 300 mM NaCl it was about 50 minutes (FIG. 4). It appears that dissociation may not follow a strictly first-order rate law; it seems to be faster at the start and slows down after a few hours (FIG. 5). This may be due to the heterogeneity of the surface, which may contain a population of immobilized Tus molecules with sub-optimal binding to BFTerB.

### 1.3 Conclusion

Here we show that the binding parameters of surfaces derived from a CM5 chip coated with Tus are comparable with those of a SA surface, with the advantage that the surface can be easily regenerated. This is not readily achieved using SA chips. A longer immobilization time of BFTerB is preferred for the SA surface than for the Tus surface; a two molar excess of immobilized SA compared to Tus is preferred to achieve surfaces comparably active in binding BFTerB. The surface is very stable and does not decay substantially over time when RB1 is used for regeneration. These data demonstrate that the surface is robust and needs lower quantities of oligonucleotides compared to SA and more importantly, there is little need for expensive biotinylation to achieve the immobilization of an oligonucleotide onto a surface. The stability of the Tus surface and Tus-bound oligonucleotide surfaces is sufficient for BIAcore applications and/or microarrays. Another advantage of the Tus surface is the possibility to inexpensively label or lengthen either one or the other strand of the forked TerB, therefore making it possible to display at once either the 5′ or 3′ termini. The Tus-forked Ter (TT-lock) nucleic acids disclosed herein are suitable to many kinds of surface display.

### Example 2

### Forked Versions of Ter Sites that Form Stable Complexes with Tus Polypeptide

In E. coli and a few other species, the terminus contains 23-bp Tus-binding (Ter) sites arranged in two groups in opposite polarity (FIG. 6). Ten highly conserved E. coli Ter sites (FIG. 6) have been described which include residues specifically implicated in fork arrest, in particular a G-C base pair at position 6 of TerB (FIG. 6), and the side chains of Glu47 and Glu49 of Tus that are located nearby in loop L1 near the non-permissive face in the structure of the complex.

In this example, the kinetics of dissociation of Tus from a series of forked variants of TerB were determined to identify preferred double-stranded nucleic acids of the invention suitable for display applications.

### 2.1 Materials and Methods

### 2.1.1 Tus Protein and Oligonucleoides

N-terminally His₆-tagged Tus was prepared as described by Neylon et al., Biochemistry 39, 11989-11999, 2000, and dialyzed into storage buffer buffer (50 mM Tris.HCl at pH 7.5, containing 0.1 M NaCl, 1 mM EDTA, 1 mM dithiothreitol and 20% w/v glycerol). The concentration of Tus protein was determined spectrophotometrically, using ε₂₈₀=39,700 M⁻¹ cm⁻¹. Before use, aliquots of Tus were freshly diluted at 0° C. into binding buffer (50 mM Tris.HCl at pH 7.5, containing 0.25 M KCl, 0.1 mM EDTA, 0.1 mM dithiothreitol and 0.005% surfactant P-20).

Oligonucleotides, some of which (as specified) were modified at the 5′ end by a biotin residue followed by a 10-mer abasic poly(deoxyribose-5′-phosphate) spacer, were from GeneWorks (Adelaide, Australia).

### 2.1.2 Surface Plasmon Resonance (SPR) Measurements

SPR measurements were carried out at 20° C. using a BIAcore 2000 instrument (Biacore AB, Uppsala, Sweden), essentially as described by Neylon et al., Biochemistry 39, 11989-11999, 2000. Two flow cells contained similar amounts of forked duplexes immobilized via one of the two 5′-biotinylated wild-type TerB strands, while the third flow-cell contained fully-double-stranded TerB (positive control) and the fourth was underivatized (blank). The amount of oligonucleotide used was sufficient to bind about 50-80 response units (RU) of Tus protein at saturating concentrations. A flow rate of 40 μL/min (Neylon et al., Biochem. 39, 11989-11999, 2000) was used for all measurements, with Tus solutions in binding buffer. Surfaces were regenerated when required with short injections (5-10 μl, at 5 μl/min) of 1 M MgCl₂ to remove bound Tus, or alternatively, 50 mM NaOH in 1 M NaCl to remove hybridized oligonucleotides. To generate new surfaces, partially complementary non-biotinylated DNA strands, as required, were annealed by injection of 20 μl of 1 μM solutions of single-stranded oligonucleotides in binding buffer. Tus does not bind to single-stranded biotinylated oligonucleotides under the conditions used. Where possible, data were fit globally to a 1:1 Langmuir binding model using BLAEvaluation software (Biacore). For the “locked” oligonucleotides, binding and dissociation phases were measured in separate experiments. Individual data sets were fit to a single exponential, giving k_{obs}. Values of kₐ were determined from the slopes of plots of k_{obs} vs [Tus].

### 2.2 Results

In summary, 21-nucleotide 5′-biotinylated TerB oligonucleotides immobilized through a 10-residue abasic spacer to streptavidin-coated chip surfaces through one or other of the two wild-type strands of TerB were produced and analysed using a Biacore 2000 instrument. The other strand contained non-complementary regions or had been shortened at either end. In this way, it was possible to examine the consequences for Tus binding of non-complementary mutated regions of various lengths on both strands at either end of TerB.

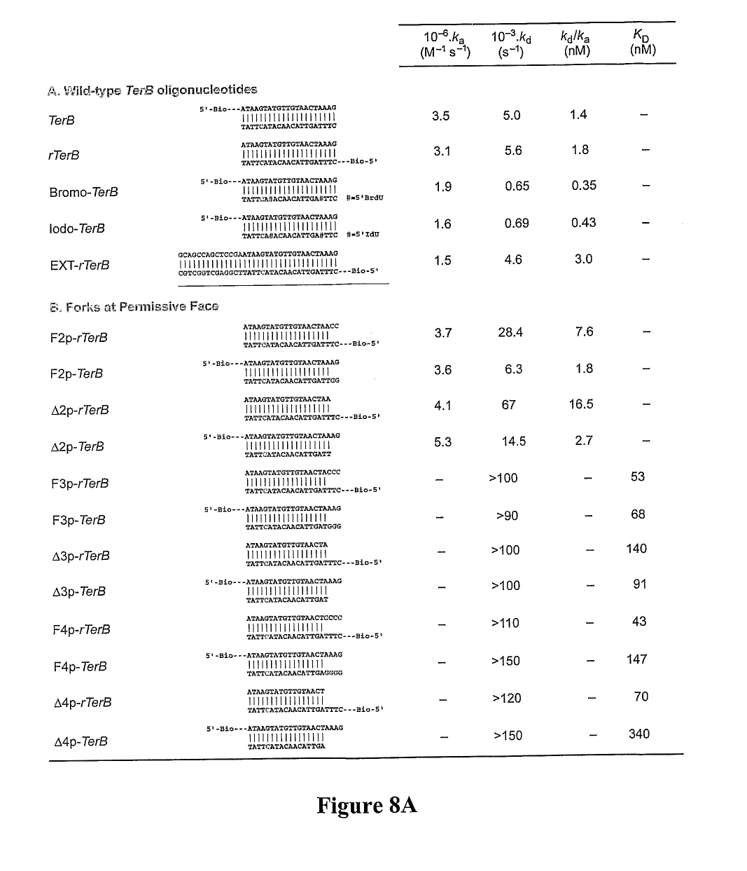

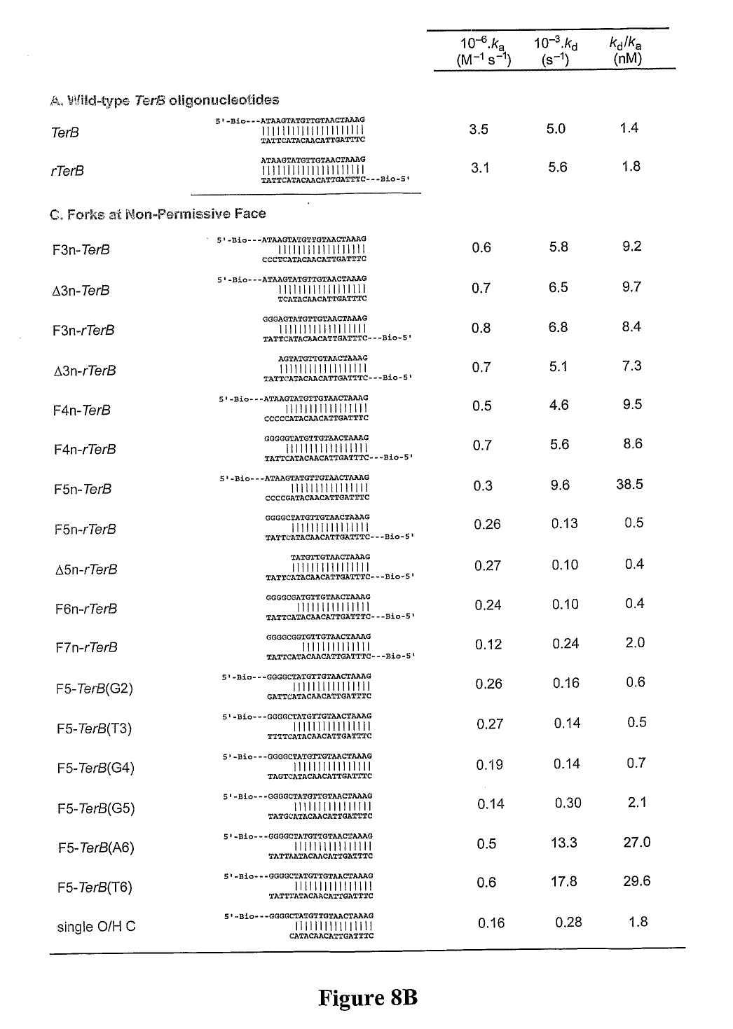

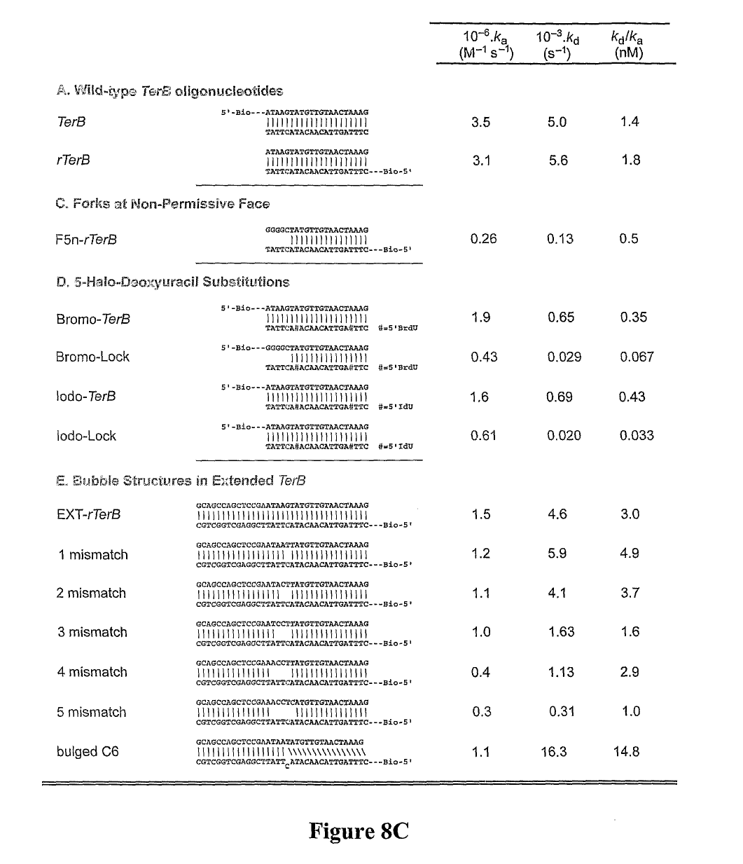

Significant BIAcore data (e.g., showing values of association and dissociation rate constants, kₐ and k_{d}, from which K_{D} was calculated as kₐ/k_{d},) are given in FIGS. 7a and 7b. Complete data and sequences of oligonucleotides are provided in FIGS. 8a and 8b.

Data for TerB and rTerB indicate that the orientation of the wild-type duplex with respect to the surface has little effect on binding parameters, and values of K_{D} were 1-2 nM under these conditions; use of 0.25 M KCl in the buffer brings these parameters into the range quantifiable using the BIAcore.

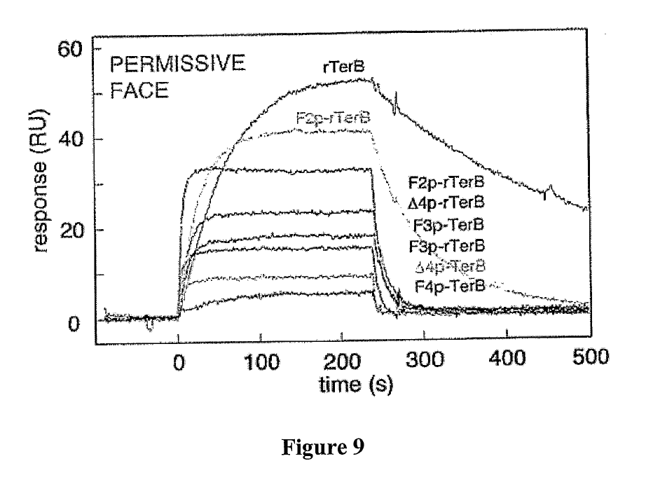

As the forked region was progressively extended at the permissive end of TerB, the value of K_{D} increased due to the dissociation rates becoming fast (FIG. 9). The data were generally consistent with progressive loss of protein-DNA contacts. If the replication-fork helicase (DnaB) were able to separate the two strands even as far as A-T(20) of TerB (FIG. 6), then it is clear Tus would dissociate rapidly to allow passage of the fork.

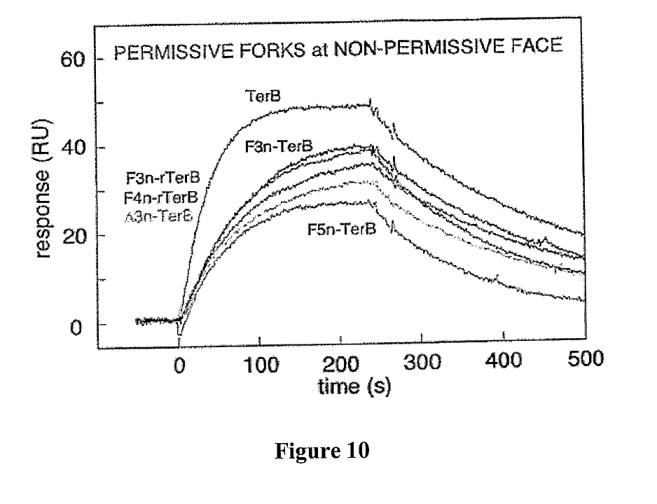

The situation was found to be different when single-stranded regions were introduced at the non-permissive end. An increase in K_{D} of up to 5-fold was observed when the single-stranded regions were 3 of 4 nucleotides long, with dissociation rates being similar to those with wild-type TerB, regardless of which strand was mutated (FIG. 10). However, strand specificity became dramatically obvious when the forked region extended to include the G-C(6) base pair. When the strand containing C(6) was mutated (the bottom strand in FIG. 7; oligonucleotide F5n-TerB; FIGS. 7 and 10), Tus was observed to dissociate more rapidly than from TerB.

On the other hand, mutation of the top strand (F5n-rTerB) resulted in Tus being firmly locked onto the forked TerB (FIG. 11): the complex dissociated about 50-fold more slowly than that with TerB, and the dissociation constant was at least 5-fold lower. Although extension of the fork to include T-A(7) resulted in similar “locked” behavior, its further lengthening to A-T(8) resulted in poorer binding due to a very slow association rate (FIG. 7).

Without being bound by any theory or mode of action, strand separation by a helicase approaching from the permissive face of the Tus-TerB complex may promote its dissociation, while at the non-permissive face helicase action would lead to a “locked” complex that dissociates some 50-fold more slowly. This explains the polarity observed in replication termination.

The strictly conserved cytosine on the bottom strand of the TerB sequence may also be involved and preferably this is not base paired for “locking” of the complex to occur. For example, the “locking” behaviour was still observed when the first five residues of the mutant strand in F5n-rTerB were completely removed (Δ5n-rTerB; FIGS. 7 and 11), indicating that a forked structure is not required provided C(6) is not basepaired, and systematic mutagenesis of each of the first five residues of the wild-type strand of F5n-rTerB up to and including C(6) showed that only mutagenesis of C(6) abrogated the “locking” behaviour of the Tus-TerB complex (FIG. 12).

To obtain a comparison of dissociation rates in a physiological buffer at 20° C., dissociation of Tus from TerB and F5n-rTerB was followed simultaneously in buffer containing 0.15 M KGIu in place of 0.25 M KCl over 18 h. Estimates of half-lives were −42 h for TerB and −130 h for F5n-rTerB.

Without being bound by any theory or mode of action, these data indicate that a replication fork approaching from the non-permissive face is blocked by a molecular mousetrap, which is set by binding of Tus to a Ter site, and sprung by strand separation by DnaB, thereby causing flipping of the conserved C residue out of the double helix by rotation of the phosphodiester backbone, and its base-specific binding in an appropriately positioned pocket in the DNA binding channel of Tus. Other contacts of Tus with the displaced strand may occur, but they are not sequence specific. This mechanism explains the observation that mutagenesis of the G-C(6) base pair of TerB compromises fork arrest without affecting Tus binding. It also explains how Tus-Ter can force a polar block on the actions of RNA polymerase and several different helicases, since these enzymes are all involved in strand separation. Specific physical interaction of DnaB with Tus is not precluded, but would appear to be unnecessary.

### Example 3

### Improved Ribosome Display and In Vitro Directed Molecular Evolution of Protein Function

Darwinian evolution relies absolutely on the physical linkage within the confines of a whole organism, cell, or viral particle of its genotype (genome, comprising sequences of DNA and/or RNA) and its phenotype (aggregate of properties of its gene products). This same principle is used for evolution of new protein functions.

The embodiments described herein provide advantages over standard methods of in vitro display e.g., reducing instability of the ternary complex in ribosome display or ribosome inactivation display. For example, a complex of Tus and a double-stranded oligonucleotide of the present invention is used to block progress of the ribosome. To achieve this, a Tus fusion protein or conjugate comprising Tus and a protein of interest is linked to the mRNA molecule from which it is translated or the DNA molecule from which the mRNA is transcribed [a genotype:phenotype-linked (G:P) complex].

### 3.1 Plasmid Vectors

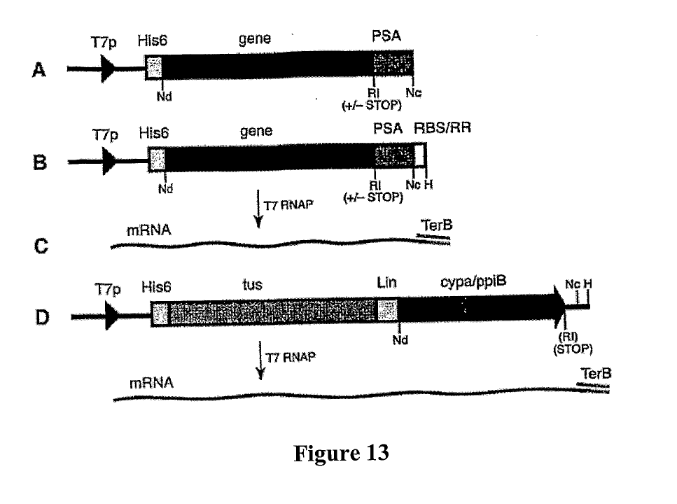

A series of plasmid vectors is constructed, as shown in FIG. 13. These are derivatives of phage T7 promoter vectors (pET derivatives) or modifications of the pEGX vectors. These vectors are designed so that mRNAs can be easily produced by runoff transcription with T7 RNA polymerase following their linearization at appropriate restriction sites. They are constructed from existing pET plasmids that contain the target genes encoding Tus, 9Ala-Tus, CyPA and PpiB, by standard techniques, e.g., linker ligation and PCR amplification with appropriate primers. In one embodiment, the plasmids comprise an EcoRI site at the 3′ end of the gene (without a stop codon) positioned relative to the reading frame so as to permit linearization with EcoRI, end-filling with DNA polymerase, and recircularization to thereby create an in frame TAA stop codon as part of a new AsnI site (denoted +/− STOP in FIG. 13).

Simple modification of the Tus-fusion vectors in FIG. 13 include TerB sites elsewhere in the vectors to enable their use for preparation of in vivo libraries for plasmid display, wherein covalent plasmid-protein G:P complexes are produced by lysis of a library of formaldehyde-treated cells. After selection, plasmids would be recovered by transformation. These plasmids could are also used for in vitro transcription and translation in artificial compartments (e.g., water-in-oil emulsions).

3.1.1 mRNA 3′-End Modifications

The prokaryotic vector constructs are modified by adding a eukaryotic ribosome-binding site with and without an upstream translational enhancer. These sequences are engineered into the prokaryotic vectors either via restriction sites or added with appropriate oligonucleotides and PCR.

For example, an E. coli translational RBS (AAGGAGGT) is added at the 3′ end of the four mRNAs (FIG. 13) between Ncol and Hpal sites, and its effect on mRNA recoveries is assessed following selection. Alternatively, a random approx. 30-nt DNA sequence is added by PCR primer extension at the Ncol site in pB-Tus and/or pB-CyPA plasmids, and used in multiple rounds of selection experiments to isolate mRNA-3′ sequences that stabilize the G:P ternary complexes. Similar experiments are performed using variants of the eukaryotic translational RBS (GCCGCCACCATGG).

3.1.2 Addition of TerB to the 3′ end of mRNAs

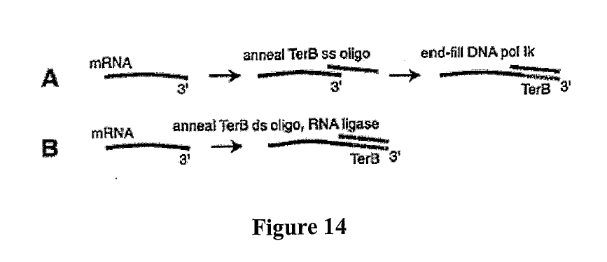

Various methods are used to attach TerB DNA to the 3′ end of mRNA. The simplest procedure, which can be monitored by incorporation of labelled dNMPs, is end-filling of a partial duplex RNA:DNA hybrid using one of a number of available DNA polymerases, as shown in FIG. 14. This improves the stability of mRNAs towards RNAse-mediated degradation. Alternatively, E. coli DNA primase is used to extend pre-existing RNA primers with dNMPs (Swart et al., Biochem. 34, 16097-16106, 1995).

A second strategy to produce 3′-TerB-mRNA is to use RNA ligase as shown in FIG. 14 (Roberts & Szostak, Proc. Natl. Acad, Sci USA 94, 12297-12302, 1997).

A third strategy to produce 3′TerB-mRNA is to use UV crosslinking with a psoralen-substituted complementary oligonucleotide pair (Kurz et al., Chem Biochem 2, 666-672, 2001).

Once double-stranded TerB DNA has been attached through one strand at the 3′ end of the mRNA, the other can be removed and replaced with another partially-complementary oligonucleotide to produce the TT-Lock of the invention, e.g., by heating 3′TerB-mRNA in the presence of excess of the second strand and slowly cooling the mixture.

### 3.2 Methods for Creation of G:P Complexes

Diversity is introduced into population of RNA/DNA molecules by creation of a library of variant genes e.g., as described by Irving et al., J. Immunol. Methods 248, 31-45, 2001.

### 3.2.1 Ribosome Display

In ribosome display, libraries of mRNA molecules encoding Tus fusion proteins (Tus conjugates) are translated in vitro under conditions where the ribosome stalls after translation of a protein molecule, thereby producing a ternary G:P complex containing the ribosome, the nascent fully-folded protein and the mRNA molecule that encodes it.

To provide improved display technologies, Tus fusion protein systems are produced e.g., near-covalent or covalent mRNA-protein fusions which are able to be translated using supplemented prokaryotic (S-30) extracts (Guignard et al., FEBS Lett. 524, 159-162, 2002), or rabbit reticulocyte lysates (Irving et al., J. Immunol. Methods 248, 31-45, 2001; Coia et al., J. Immunol. Methods, 254, 191-197, 2001) are used for in vitro protein expression and ribosome display or ribosome inactivation display.

Allowing translation to occur right to the end of a mRNA that contains no in-frame stop codon efficiently prevents dissociation of the nascent polypeptide, and provided that it is extended by a C-terminal tail greater than about 30 amino acid residues in length, the polypeptide folds into its active native structure (Kudlicki et al., Biochem. 34, 14284-14287, 1995; Makeyev et al., FEBS Lett., 378, 166-170, 1996).

### 3.2.2 Ribosome Stalling Using a Tis-TerB Block

3′-TerB-mRNAs (four test genes, FIG. 13, —STOP), each bound by purified His₆-Tus (Neylon et al, Biochem. 39, 11989-11999, 2000) and the complexes are detected e.g., by immunoprecipitation or blotting of RNA gels with anti-His₆ or anti-Tus antibodies or binding to Ni-NTA. Ribosome display competition experiments are carried out, and mRNA recoveries quantified by real-time PCR. The effect of orientation of TerB is examined since the Tus-TerB complex arrests other macromolecular assemblies such as the replisome and/or RNA polymerase in a polar manner. Tus has many basic residues in its DNA-binding pocket making it feasible to reversibly crosslink Tus to the double-stranded nucleic acid of the invention using formaldehyde, thereby enhancing the stability of the complex under conditions of high ionic strength where it normally dissociates rapidly.

### 3.2.3 Protein-mRNA Fusions

The protein-synthesis inhibitor puromycin is covalently attached at the 3′ end of mRNA molecules in the library, essentially as described by Roberts & Szostak, Proc. Natl. Acad, Sci USA 94, 12297-12302, 1997 or Nemoto et al., FEBS Lett. 414, 405-408, 1997. During translation, the puromycin moiety enters the A site of the ribosome and, like puromycin itself, forms a stable covalent peptidyl-tRNA analog that dissociates from it. Because the puromycin is linked to the mRNA, this results in ribosome-promoted formation of protein-puromycin-mRNA conjugates that can be stabilized by conversion to cDNA, stored indefinitely and screened. Because ribosomes act as catalysts rather than stoichiometric reagents, G:P libraries with >1014 members are created.

The 3′-TerB tagged mRNAs are used to bind in vitro-synthesized Tus in cis, to produce non-covalent mRNA protein fusions that are then converted to covalent mRNA-protein fusions by formaldehyde crosslinking. The translated mRNAs comprise stop codons, to permit ribosomes to recycle, thereby increasing potential library sizes.

### 3.2.4 Selection Methods

To select for an evolved function, e.g., modified enzyme function or ligand-binding specificity, a ligand is immobilized on a solid surface (or bead) and the higher affinity or tighter-binding G:P complexes are selected by panning. The degree of selectivity in the binding reaction does not need to be especially high, because repeated cycles of creation of the G:P complexes and panning (i.e., an “evolution cycle”) can be used to purify these higher affinity-binding proteins and their genes away from those binding more weakly, in addition to allowing rounds of further limited mutagenesis (affinity maturation). The G:P complexes are stable during selection since their stability limits the effective size of the library that is screened in each round of selection.

Selection systems are used to quantitatively probe the efficiency of formation and the stability of G:P complexes, using selection systems that rely on binding of Tus proteins to the double-stranded oligonucleotide of the invention immobilized on streptavidin-coated magnetic beads or surfaces.

For example, selections using Tus vs. 9Ala-Tus and CyPA vs. PpiB are performed as described below.

### 3.2.5 Ribosome-Display Selections

Plasmids with inserts shown in FIG. 13 (pA plasmids) with each of the four genes (encoding Tus and 9Ala-Tus, CyPA and PpiB, each +/− STOP) are tested in ribosome-display competition experiments to determine selectivity. The mRNA composition of G:P ternary complexes recovered on beads during selection is quantitatively examined by use of real-time RT-PCR using primers specific for each of the genes. Ribosomal RNA is also quantified, and quantitative measurements of the mRNA:ribosome ratio are obtained. The anticipated reduction of mRNA recovery on inclusion of stop codons is verified.

For example, ribosome-display competition experiments were used to examine the selectivity for wild-type over mutant versions of Tus in binding to TerB: the two mRNAs were mixed in different proportions and used to program protein synthesis. Ternary G:P complexes were selected on TerB-coated beads, and the bound message recovered by RT-PCR. Restriction digests that distinguished between the two templates were then used to determine the relative amounts of the mRNAs recovered. This gave a direct measure of selectivity for wild-type vs. mutant Tus. With Q250A mutant Tus, which binds TerB 100-fold less tightly than the wild-type (Neylon et al., 2000), single-round selectivity for the wild-type Tus was shown to be 10- to 20-fold.

Site-specific DNA-binding by Tus is ablated using a mutant form in which eight residues that make specific DNA contacts in the Tus-Ter complex have been converted to alanine residues (9Ala-Tus).

### 3.2.6 Selection of Functional Tus Using 3′-TerB-mRNA

In vitro translation of the Tus-mRNAs in FIG. 13 or 14 (Tus/9Ala-Tus; + STOP), once 3′-tailed by TerB, is used for selection of functional Tus. Although the Tus will dissociate from the ribosome on completion of its synthesis, the proximity of TerB ensures that it binds predominantly in cis to the message from which it was synthesized. The reaction produces tight non-covalent mRNA-protein conjugates that are produced catalytically by the ribosomes, and preferably stabilized by formaldehyde crosslinking.

Protein-RNA conjugates are isolated, the mRNA amplified by RT-PCR or RNA replication, and recycled to the next round of enrichment. This approach is used to evolve DNA-binding specificity of Tus or other (monomeric) DNA-binding proteins.

A library of Tus variants wherein nine site-specific DNA-binding residues have been randomized (library size about 5×10¹¹) has been produced using such approaches. This library is used for first-round screening for Tus variants that bind to TerB or other nucleic acids described herein, and binding is optimized by affinity maturation using technology known in the art. Specific 5′- and 3′-nucleotide sequences are added to the vector constructs to permit the RNA transcribed from the gene within the display cassette to be mutagenized and directly shunted into ribosome display without any intermediate steps.

### 3.2.7 Cyclophilin-Cyclosporin

This selection system uses two closely-related cyclophilin-type peptidyl-prolyl cis-trans isomerases: E. coli PpiB (Edwards et al., J. Mol. Biol. 271, 258-265, 1997) and human cyclophilin A (CyPA). The human protein binds tightly (K_{D} about 6 nM) to the cyclic peptide drug cyclosporin A (CsA), while binding of the bacterial protein is about 3000-fold weaker (Liu et al., Biochem. 30, 2306-2310, 1991). As ligand, a CsA derivative is used that contains a D-lysine residue remote from the CyPA-binding site (Novartis Research Labs). The D-Lys side chain is biotinylated and attached to beads or to a streptavidin-coated BIACore chip to study protein interactions.

In ribosome-display competition experiments, single-round selectivity for binding of CsA-coated beads by CyPA over PpiB was shown to be −20-fold.

### 3.2.8 Selection of Other Proteins Using 3′-TerB-mRNA: Tus Fusion Complexes:

The above approach is further generalized for selection of binding specificities in other proteins by using Tus-gene fusions (see FIG. 13). The fusion protein is translated in vitro, and the Tus moiety folds and binds (as a monomer) in cis to the TerB sequence at the 3′ end of the mRNA that encodes it. The remainder of the message is translated to a stop codon at the end of the target gene (or library of genes), and the fusion protein-mRNA conjugate dissociates from the ribosome, allowing it to recycle to new mRNAs. Following translation, the non-covalent protein-mRNA conjugates can be stabilized further by reversible formaldehyde crosslinking, and used for selection experiments. Crosslinks in recovered G:P complexes are ‘reversed’ and the RNA amplified as before for a new cycle of enrichment/evolution.

For example, ribosome display is also used to identify variant sequences of an antibody-like molecule (12Y-2) that binds to a malarial specific protein (apical membrane antigen 1 [AMA-1]) with a moderate affinity. The 12Y-2 sequence is cloned into the display cassette followed by in vitro translation, panning for increased binding to AMA-1, and RT-PCR to show the specific recovery of binding fragments.

### 3.3 Amplification

Following selection, the mRNA is amplified for further rounds of maturation/enrichment either by reverse transcription-PCR, followed by transcription or by RNA replication (Irving et al., 2001). In principle, accessible library sizes for screening are limited only by the numbers of active ribosomes in the translation reaction, which can be about 1×10¹².

### Example 4

### Application of the TT-Lock to a New Regenerable Surface Plasmon Resonance Chip to Monitor Direct Real-Time Kinetics of Nucleases

A DNA segment that encodes an 18-residue biotin-tag sequence (MAGLNDIFEAOKIEWHEH) was fused to the tus gene to provide a specific mono-biotinylation site on the N-terminus of Tus.

Two oligonucleotides: (5′-TAATGGCTGGTCTGAACGACATCTTCGAAGCTCAGAAAATCGAATGGCACGA ACATATGA (SEQ ID NO: 75) and (5′-CGCGTCATATGTTCGTGCCATTCGATTTTCTGAGCTTCGAAGATGTCGTTCAG ACCAGCCAT (SEQ ID NO: 76) (NdeI site underlined) were annealed and ligated between the NdeI and MluI sites in the phage T7-promoter vector pETMCSI (Neylon et al., (2000) Biochemistry 39, 11989-11999) to produce plasmid vector pKO1274. The tus gene from pCM847 (Neylon et al., (2000) Biochemistry 39, 11989-11999) was subsequently ligated between the NdeI and EcoRI sites in pKO1274 to produce pKO1285. The biotinylated Tus (Bio-Tus) was produced in E. coli strain BL21:DE3/pLysS at room temperature using an auto-induction medium (Studier (2005) Protein Expr Purif 41, 207-234) Bio-Tus was purified following a method developed for wild-type Tus (Neylon et al., (2000) Biochemistry 39, 11989-11999).

The E. coli strain BL21:DE3/pLysS/pSH1018 (Hamdan et al., (2002) Biochemistry 41, 5266-5275) was used to overproduce ε186, also using an auto-induction system (Studier (2005) Protein Expr Purif 41, 207-234). The procedure for purification of ε186 essentially followed that described in (Hamdan et al., (2002) Biochemistry 41, 5266-5275).

### 4.1 Oligonucleotides for Exonuclease Assay

All experiments were carried out at 20° C. and at a flow rate of 5 μL/min in a Biacore 2000 instrument (Biacore AB, Uppsala, Sweden) equilibrated with Biacore buffer (10 mM HEPES pH 7.55, 3 mM EDTA, 1 mM dithiothreitol, 150 mM NaCl, 4 mM MgCl₂) unless otherwise stated. The oligonucleotides used for the exonuclease assay were annealed and diluted in HBS-P buffer (Biacore). Sequences of oligonucleotides were:

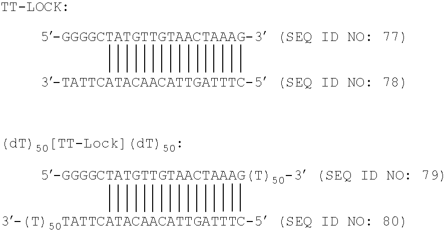

### 4.2 Preparation of the Bio-Tus Chip

A SA chip (Biacore) was activated according to the manufacturer's guidelines. Bio-Tus was diluted in HBS-P and immobilized onto the flow cells during a 4 min injection to yield an increase of 5000 response units (RU). After a short stabilization period, different combinations of oligonucleotides were injected over the flow cells to check the activity of the Bio-Tus. Regeneration of the chip was achieved successfully using a 1 min pulse injection of 1 M MgCl₂. Only the bound oligonucleotides were eluted under these mild conditions.

### 4.3 Exonuclease Assay

The ε186 was diluted in Biacore buffer and the Biacore 2000 instrument was equilibrated with the same buffer. Kinetic measurements of the exonuclease activity of ε186 were monitored after injection of (dT)₅₀-[TT-Lock](dT)₅₀ oligonucleotide substrate (1 μM, in HBS-P) during 1 min at a flow rate of 5 μl/min over the Bio-Tus surface. After a stabilization period with Biacore buffer (6 min, 20 μl/min), ε186 solutions were injected during 4 min at the same flow rate. The surface was regenerated and this process was repeated with various concentrations of 8186.

### 4.4 Results and Discussion

### 4.4.1 Preparation of a Regenerable Oligonucleotide Binding Surface

Use of a biotinylated Tus that could be immobilized onto SA chips in only one orientation resulted in approximately 5000 RU of Bio-Tus immobilized onto a SA chip surface (data not shown). This surface was able to bind between 300 and 500 RU of TT-Lock and was fully regenerable with a pulse injection of 1 M MgCl₂. The Bio-Tus chip was stable when challenged with multiple regeneration and binding steps over a period of several hours. The Bio-Tus surface also showed no non-specific (i.e., DNA independent) interaction with ε186.

### 4.4.2 Exonuclease Assay

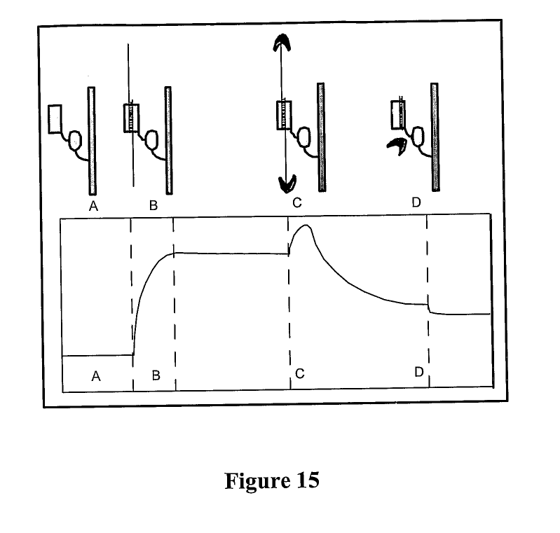

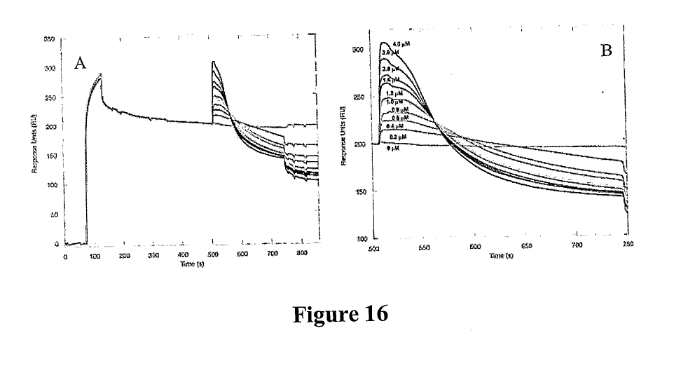

A model representation of the progress of a real-time exonuclease assay is shown in FIG. 15. When the (dT)₅₀[TT-Lock](dT)₅₀ oligonucleotide that exposes two single-stranded (dT)₅₀ arms was injected over the Bio-Tus:SA surface, the SPR signal increased sharply and the baseline stabilized within a few minutes, as shown in FIG. 16. The (dT)₅₀[TT-Lock](dT)₅₀ substrate was designed to double the response compared to an oligonucleotide with only one single-stranded DNA arm, and therefore to increase the signal-to-noise ratio. Upon injection of 186, a binding event was observed followed by a sharp, approximately linear loss of signal as the exonuclease activity of the enzyme resulted in progressive removal of the single-stranded DNA from the surface (FIG. 15). A set of ten different concentrations of 6186 ranging from 0.2 to 4 μM were tested in the same way and the corresponding sensorgrams are overlaid in FIG. 16. When this experiment was carried out in the absence of MnCl₂, the initial binding of the enzyme was observed, but no loss of signal occurred subsequently, confirming the absolute requirement of divalent metal ions for the exonuclease activity (data not shown).

Under ideal conditions, data in FIG. 16 can be used to determine values of both K_{M} and k_{cat}. The dependence of the slope (v) on [enzyme] gives a Michaelis-Menten curve, from which k_{cat} (maximum rate in Nt/min/active site) and K_{M} ([enzyme] at half-maximal rate) may be determined. Extrapolation of the initial binding data to zero time gives, in principle, data for calculation of the dissociation constant of the enzyme-substrate (Michaelis) complex (K_{S}).

### 4.5 Conclusion

The inventors have demonstrated the utility of the TT-Lock technology as a reversible but stable oligonucleotide immobilization technique for the conception of a real-time SPR-based exonuclease assay. This assay can be applied to the study of any exo- or endonuclease activities and any assay or technology with a need for a reversibly immobilized single- or double-stranded oligonucleotide.

Alternative assays cannot be easily achieved by annealing a complementary strand of DNA to a previously immobilized oligonucleotide, given that it is difficult to achieve rapid annealing of oligonucleotides at room temperature without very high concentrations of the complementary strand. Furthermore, a proportion of the immobilized oligonucleotides will always be free and prone to be degraded (resulting in irreversible destruction of the template surface), especially if relatively crude sources of enzymes are used (as is the case, for example, in functional genomics applications).

Another significant advantage of the technique is the fact that the double-stranded TT-Lock portion of the oligonucleotides are protected by Tus, which literally wraps around them.

Finally, only the specific forked DNA sequence of the TT-Lock will bind to this surface, rendering it free of any non-specifically-bound DNA that might otherwise complicate assays.

### Example 5

### Determination of Polarity of Termination of DNA Replication

### 5.1 Introduction

During chromosome synthesis in Escherichia coli, replication forks are blocked by Tus-bound Ter sites on approach from one direction, but not the other. To study the basis of this polarity, the inventors measured the rates of dissociation of Tus from forked TerB oligonucleotides such as would be produced by the replicative DnaB helicase, at both the fork-blocking (non-permissive) and permissive ends of the Ter site. Strand separation of a few nucleotides at the permissive end was sufficient to force rapid dissociation of Tus to allow fork progression. In contrast, strand separation extending to and including the strictly-conserved G-C(6) base pair at the non-permissive end led to formation of a stable “locked” complex. “Lock” formation specifically requires the cytosine residue, C(6). The crystal structure of the “locked” complex showed that C(6) moves 14 Å from its normal position to bind in a cytosine-specific pocket on the surface of Tus.

These findings were based on the hypothesis that approach of DnaB, at the forefront of the replisome, to a Tus-Ter complex engineers a structure in DNA that differentially affects dissociation of Tus depending on the direction of its approach. By examining the rates of dissociation of Tus from forked variants of TerB (that mimic structures that would be produced by helicase action), the results show that the rates of dissociation of Tus from forked TerB oligonucleotides are profoundly different depending on whether the fork is at the permissive or the non-permissive face. In particular, forks that expose the strictly-conserved G-C(6) base pair at the non-permissive face produce a complex in which Tus is “locked” onto the DNA: It dissociates about 40-fold more slowly than from wild-type TerB. This “locking” behavior was then traced to a single nucleotide base (C6) of Ter, which it appears must form a new contact with a cryptic cytosine-specific single-stranded DNA-binding site on the surface of Tus. This behaviour of C6 was confirmed by means of an X-ray crystal structure of Tus in complex with an appropriate forked duplex version of Ter.

Experiments indicating that the Tus-forked Ter complex is a kinetic rather than a thermodynamic “lock” were then undertaken, thus offering a plausible explanation for the necessity for multiple oppositely-oriented Ter sites on each arm of the bacterial chromosome, as shown in FIG. 6A.

Finally, it was investigated as to what may happen when the later-arriving, oppositely-moving replisome approaches the first stalled at the Tus-Ter complex. In this regard, it was shown that strand separation at the permissive face can “unlock” the first complex, displacing Tus to allow replication of the remaining double-stranded DNA at the terminus.

### 5.2 Experimental Procedures

### 5.2.1 Tus Protein and Oligonucleotides

Tus and N-terminally His₆-tagged Tus was prepared as described (Neylon et al. (2000) Biochemistry 39, 11989-11999), with concentrations determined spectrophotometrically (ε₂₈₀=39,700 M⁻¹ cm⁻¹). Oligonucleotides, some of which (as specified) were modified at the 5′ end by a biotin residue followed by a 10-mer abasic poly(deoxyribose-5′-phosphate) spacer, were from GeneWorks (Adelaide, Australia). Sequences of all oligonucleotides are given in FIG. 8.

### 5.2.2 Surface Plasmon Resonance (SPR)

Before use, aliquots of His₆-Tus were freshly diluted at 0° C. into SPR binding buffer (50 mM Tris.HCl at pH 7.5, containing 0.25 M KCl, 0.1 mM EDTA, 0.1 mM dithiothreitol and 0.005% surfactant P-20). SPR measurements were carried out at 20° C. using a Biacore 2000 instrument (Biacore AB, Uppsala, Sweden), essentially as described (Neylon et al. (2000) Biochemistry 39, 11989-11999). Two flow cells contained similar amounts of forked duplexes immobilized via one of the two 5′-biotinylated wild-type TerB strands, while the third flow-cell contained fully-double-stranded TerB (positive control) and the fourth was underivatized (blank). The amount of oligonucleotide was sufficient to bind 25-50 response units (RU) of Tus at saturating concentrations. A flow rate of 40 μl/min (Neylon et al. (2000) Biochemistry 39, 11989-11999) was used for all measurements, with Tus solutions at 5-10 different concentrations in SPR binding buffer. Surfaces were regenerated when required with short injections (1-2 min, at 5 μl/min) of 50 mM NaOH in 1 M NaCl. This was shown to be sufficient to remove the annealed non-biotinylated DNA strands along with any tightly-bound Tus. To generate new DNA surfaces, partially complementary non-biotinylated DNA strands were annealed by injection of 20 μl of 1-1M solutions of single-stranded oligonucleotides in SPR binding buffer. Tus does not bind to single-stranded biotinylated oligonucleotides under the conditions of these experiments (Neylon et al. (2000) Biochemistry 39, 11989-11999). When required, injection of 1 M MgCl₂ (2 min, at 5 μl/min) was sufficient to remove just Tus, leaving the oligonucleotides undisturbed. When dissociation rates were fast, data were globally fit to a 1:1 Langmuir binding model using BIAEvaluation software (Biacore). When rates were slow (i.e., with the complex in the “locked” configuration), the association and dissociation phases were studied separately. Second-order association rate constants (kₐ) were obtained as slopes of plots of pseudo-first-order rate constants (k_{obs}) versus concentration of Tus, and values of k_{d}, the dissociation rate constant, were obtained directly by fitting to a first-order rate law. The error in all reported parameters was less than 10%.

### 5.2.3 Dissociation Rates of Tus-Ter Complexes in Solution

The half-lives of complexes of His₆-Tus with TerB oligonucleotides (Table 3) were measured essentially as described (Skokotas et al., (1995) J. Biol. Chem. 270, 30941-30948). ³²P-labeled Ter DNA (0.05 nM) was equilibrated with Tus (0.25 nM) at 25° C. in 50 mM Tris.HCl at pH 7.5, containing 0.20 M potassium glutamate, 0.1 mM EDTA, 0.1 mM dithiothreitol and 100 μg/ml bovine serum albumin (KG₂₀₀ buffer). Excess unlabeled wild-type TerB oligonucleotide (5 nM) was added as a trap to bind dissociated Tus. Samples were removed periodically and applied to nitrocelloulose filters, which were washed with KG₂₀₀ buffer, dried and counted in a scintillation counter.

### 5.2.4 Structure Determination

HPLC-purified “lock” oligonucleotides 5′-TTAGTTACAACATACT (SEQ ID NO: 81) and 5′-TGATATGTTGTAACTA (SEQ ID NO: 82) were combined at 0.3 mM each in 25 mM Bis-Tris at pH 6.2 containing 100 mM NaCl, 1 mM EDTA and 1 mM dithiothreitol, and annealed by slow cooling from 70° C. To this mixture (0.25 ml) was added Tus (0.25 ml at 0.25 mM, in 50 mM sodium phosphate, pH 6.8, containing 50 mM NaCl, 0.1 mM EDTA and 1 mM dithiothreitol). After 5 min at 20° C., the complex was diluted to 5 ml with 10 mM Bis-Tris at pH 6.3, containing 1 mM EDTA and 1 mM dithiothreitol and then concentrated to 0.5 ml using an Amicon Ultra 15 centrifugal filter (MWCO 10 kDa). Dilution and concentration steps were repeated three times.

This “Tus-Ter lock” complex was crystallized by vapor diffusion at 18° C. from hanging drops in 24-well trays. Reservoir solution (1 ml) consisting of 50 mM Bis-Tris buffer at pH 6.75, containing 13% PEG 3350 and 0.2 M NaI, was equilibrated with a hanging drop of 4 μl of the complex mixed with 4 μl of reservoir solution. Bipyramidal crystals appeared after 1 week, and grew to a maximum size (0.2×0.2×0.4 mm) after 3 weeks. These crystals diffracted X-rays to 3.5 Å. Diffraction quality was improved by transferring crystals to artificial mother liquors with progressively increasing PEG 3350 concentrations; [PEG] was increased in 2.5% steps to a final concentration of 35% over 4-min intervals, giving X-ray diffraction to 2.7 Å resolution. Crystals were snap frozen at 100 K using an Oxford N₂ cryostream, and X-ray data were collected using a MAR345 image plate detector and goniostat system (Marresearch) using Cu Kα X-rays (λ=1.5418 Å) from a Rigaku RU-200 (80 mA, 48 kV) rotating-anode generator with 300 μm focus Osmic blue optics (NSC Rigaku). Diffraction data were integrated and scaled using the DENZO and SCALEPACK programs from the HKL suite (Otwinowsld, Z. (1993). Proceedings of the CCP4 Study Weekend, 29-30 Jan. 1993, L. Sawyer, N. Isaacs, and S. Bailey, eds (Warrington, UK: Daresbury Laboratory) pp. 56-62).

The structure was solved by molecular replacement using the MOLREP package (Vagin and Teplyalcov (1997) J. Appl. Crystallogr. 30, 1022-1025) and the coordinates of the Tus-TerA complex (Kamada et al., (1996) Nature 383, 598603). It was revealed that the crystals were of the same space group as those obtained for the Tus-TerA complex (P4₁2₁2), and the molecular replacement solution corresponded to the highest peaks from rotation and translation functions (7.54σ and 45.2σ, respectively). Model building and refinements were carried out using REFMAC5 (Murshudov et al., (1997) Acta Crystallogr. D53, 240255) and O (Jones et al., (1991) Acta Crystallogr. A47, 110-119). A randomly-selected set of 5% of the reflections were used to calculate free-R factors and validate the refinement strategy.

### 5.3 Results and Discussion

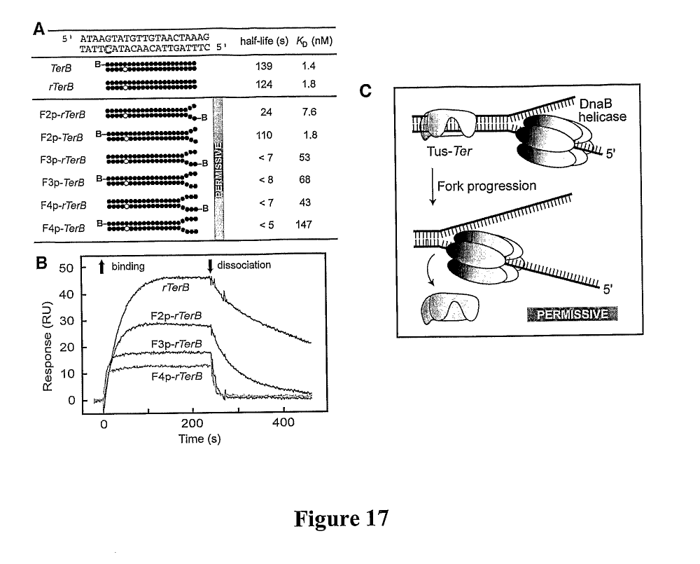

The kinetics and thermodynamics of interaction of Tus with TerB and forked versions of it were studied first by surface plasmon resonance (SPR), using a Biacore 2000 instrument, at 20° C. in a buffer at pH 7.5 containing 250 mM KCl; 21-nucleotide 5′-biotinylated TerB oligonucleotides were immobilized through an abasic spacer to streptavidin-coated SPR (Biacore) chip surfaces, essentially as described previously (Neylon et al. (2000) Biochemistry 39, 11989-11999). Each of the strands of TerB was immobilized separately, and the other (hybridized) strand contained non-complementary regions (e.g., as shown in FIG. 17A). Examination of dissociation of Tus from TerB sites containing non-complementary mutated regions of various lengths on both strands at each end could therefore be undertaken. Dissociation generally followed a first-order rate law; half-lives and dissociation constants (K_{D}) of the wild-type complex in both orientations (TerB and rTerB) are given in FIG. 17A, with complete kinetic and thermodynamic data and sequences of these and all other oligonucleotides given in FIG. 8. The data for TerB and rTerB indicate that the orientation of the wild-type duplex with to respect to the surface had little effect on binding parameters, and values of K_{D} were 1-2 nM under these conditions.

### 5.3.1 Strand Separation at the Permissive Face of TerB Leads to Rapid Dissociation of Tus

As the forked region was progressively extended at the permissive end of TerB, dissociation rates became progressively faster, and it mattered little which strand was mutated (FIG. 17A, B) or if either of them were removed completely (FIG. 8). It was clear that strand separation even as far as A-T(20) of TerB would lead to rapid dissociation of Tus (FIG. 17A, oligonucleotides F3p-TerB and F3p-rTerB), resulting in unimpeded progression of the replisome through Ter (FIG. 17C). Although potential contacts between Tus and this region of Ter are beyond the end of the oligonucleotide used for determination of the crystal structure of the Tus-TerA complex (Kamada et al., (1996) Nature 383, 598-603), modeling reveals potential contacts of the side chains of Gln 248, Trp 243 and Arg 288 of Tus with T(21), T(20) and A-T(19) of Ter, respectively (Neylon et al., (2005) Microbiol. Mol. Biol. Rev. 69, 501-526). In fact, one (or two) of these contacts with T(20) or T(21) appeared to persist in the strand-separated complex, since mutation of this “upper” strand in FIG. 17A had a consistently greater effect on the K_{D} of the complexes than alteration of the other (see also FIG. 8). The data are thus consistent with removal of Tus due to progressive loss of protein-DNA contacts during strand separation by the helicase at the permissive end of Ter.

5.3.2 T is “Locks” onto Strand-Separated Duplexes at the Non-Permissive Face

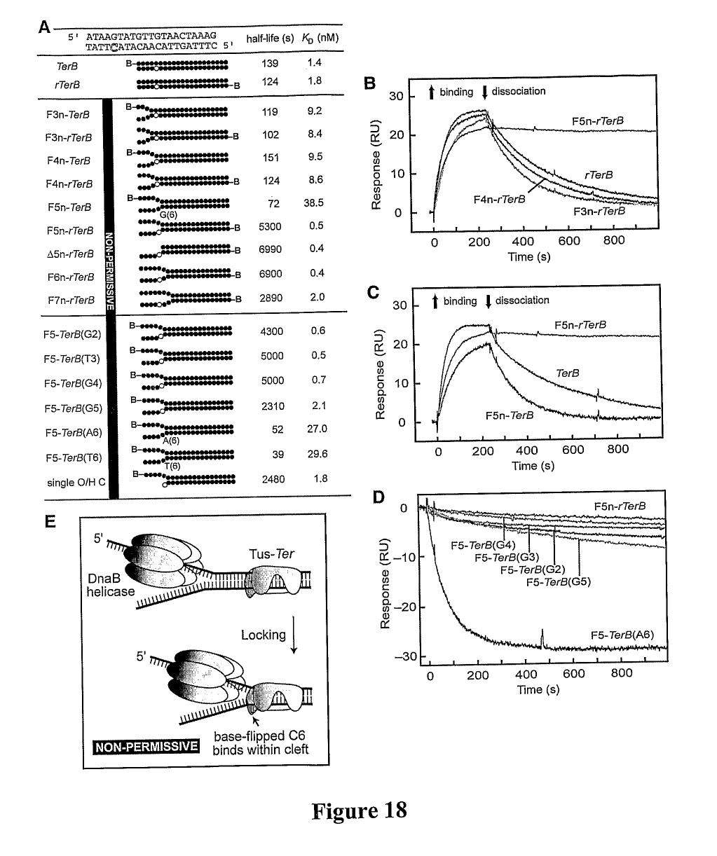

The situation was different with single-stranded regions at the non-permissive end. An increase in K_{D} of five- to seven-fold was observed when the mismatched regions were three or four nucleotides long, with dissociation rates being similar to those with wild-type TerB, regardless of which strand was mutated (FIG. 18A, B). However, strand specificity became dramatically obvious when the forked region extended to the G-C(6) base pair. When the strand containing C(6) was mutated (the bottom strand in FIG. 18A; oligonucleotide F5n-TerB), Tus was observed to dissociate about twice as rapidly as from TerB (FIG. 18A, C), and K_{D} increased almost 30-fold. On the other hand, mutation of the top strand (F5n-rTerB) resulted in Tus being firmly locked onto the forked TerB (FIG. 18A-C): Tus dissociated about 40-fold more slowly than from TerB, and K_{D} was about threefold lower. Although extension of the fork to include T-A(7) resulted in a similar “locked” behavior, its further lengthening to A-T(8) resulted in poorer binding due to a much slower association rate FIG. 18A).

Strand separation by a helicase approaching from the non-permissive face of the Tus-Ter complex would therefore lead to a “locked” complex that is even more stable than the regular complex with fully duplex TerB, while at the permissive face helicase action would simply promote dissociation of Tus. These observations provide an explanation of the polarity observed in replication termination.

### 5.3.3 A Single Nucleotide Determines Polarity of Fork Arrest

The strictly conserved C(6) base on the bottom strand of the TerB sequence in FIG. 18A must not be base paired for “locking” of the complex to occur, as verified herein. The “locking” behaviour was still observed when the first five residues of the mutant strand in F5n-rTerB were completely removed (A5n-rTerB; FIG. 18A), indicating that a forked structure is not required, and systematic mutagenesis of each of the first five residues of the wild-type strand of F5n-rTerB showed that mutagenesis of C(6), and only C(6), abrogated the “locking” behavior of the Tus-TerB complex (FIG. 18A, D). Indeed, complete removal of the first four residues on the 3′ strand, leaving only C(6), still resulted in formation of a “locked” species (“single O/H C”, FIG. 18A). The unpaired C(6) residue is thus necessary and sufficient for “lock” formation.

These data suggest that a “molecular mousetrap” operates during replication fork arrest at the non-permissive face of Tus-Ter (FIG. 18E). The trap is set by binding of Tus to the Ter site, and sprung by strand separation by DnaB at the forefront of the approaching replisome. This results in flipping of the C(6) residue out of the double helix by rotation of the phosphodiester backbone, and its base-specific binding in a cryptic cytosine-specific binding pocket in or near the DNA binding channel of Tus. Other contacts of Tus with the displaced strand may occur, but they are not sequence specific.

Specific physical interaction of DnaB with Tus is not precluded, but would appear to be unnecessary. Several further experiments were carried out to study aspects of this model.

### 5.3.4 Formation of the Tus-Ter “Lock” is Masked in Potassium Glutamate Buffers

Measurements of dissociation of Tus from TerB and partial-duplex TerB derivatives in solution were made using a filter-binding assay. Complexes of Tus with three different ³²P-labeled oligonucleotides (Table 3) were challenged with a 100-fold excess of unlabeled wild-type TerB oligonucleotide, and samples were filtered at various to times to determine the proportion of protein-bound ³²P remaining. Dissociation of Tus generally followed a first-order rate law; half-lives of the complexes are given in Table 3. It is apparent from these assays that dissociation half-lives in glutamate buffer were much more similar for the wild-type TerB oligonucleotide and those that expose C(6), indicating that the “locked” conformation of the DNA either no longer forms under these conditions, or more likely, that dissociation of Tus from it occurs at a similar rate as from wild-type TerB, i.e., existence of the “lock” is masked by the higher stability of the wild-type complex.

TABLE 3
Half-lives for Dissociation of Tus-Ter Complexes
in 200 mM Potassium Glutamate^{a}

| Oligonucleotide^{b} | Half-life (min)^{c} |
|---|---|
| 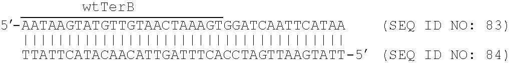 | 150 ± 6 |
| 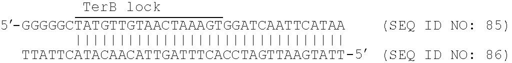 | 131 ± 7 |
| 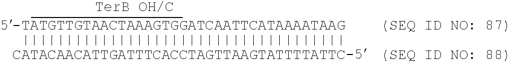 | 205 ± 8 |

^{a}Measured by a competition filter-binding assay in KG₂₀₀ buffer at 25° C. (Skokotas et al., 1995).
^{b}The core TerB sequences are overlined.
^{c}Average of 3 independent experiments (± SEM).

### 5.3.5 Ionic Strength-Dependence of the Tus-Ter Interactions

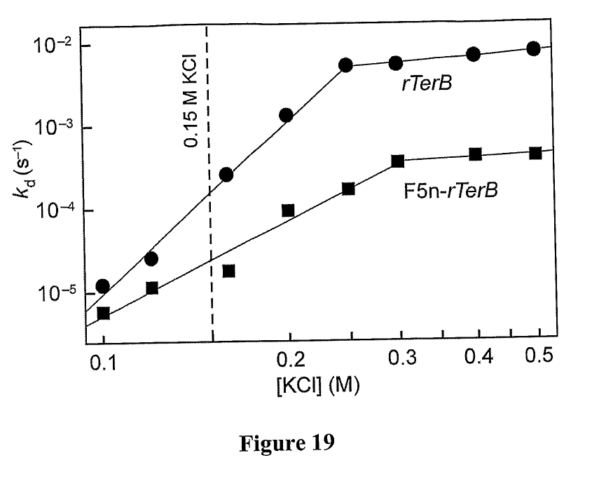

The effect of ionic strength on dissociation rate constants (k_{d}) was then measured by SPR (FIG. 19). At high ionic strength, a large-difference in k_{d} was observed for F5n-rTerB cf. rTerB, with little dependence on ionic strength. At low ionic strength, the two lines in FIG. 19 have a steeper slope and converge. The slopes of lines in such log/log plots are directly related to the numbers of ionic contacts that need to be disrupted during the rate-determining step in dissociation of a protein from a DNA complex (Record et al., (1991). Methods Enzymol. 208, 291-343). These data therefore offer further support for a stepwise mechanism for dissociation of Tus from both TerB and the forked species, and show that the rate-determining step in each process changes with ionic strength. With both oligonucleotides, the slowest step in dissociation at high ionic strength involves loss of a single (or few) ionic interaction(s), while at low ionic strength the rate-determining step requires disruption of a much larger number of such interactions. It is very likely that the slowest step in dissociation of Tus from the “locked” complex at higher salt concentrations is removal of the C(6) base from its new binding pocket, while for the wild-type complex, it is the breakage of a particular, but undetermined, site-specific interaction. At a “physiological” ionic strength corresponding to 150 mM KCl, the half-lives for the wild-type and “locked” complexes were still very different, being about 80 and 490 min, respectively (FIG. 19). Thus, the more stable “locked” species would be expected to be generated by the action of DnaB under intracellular conditions.

### 5.3.6 Tus Maintains Base-Specific Contacts in the “Locked” Complex

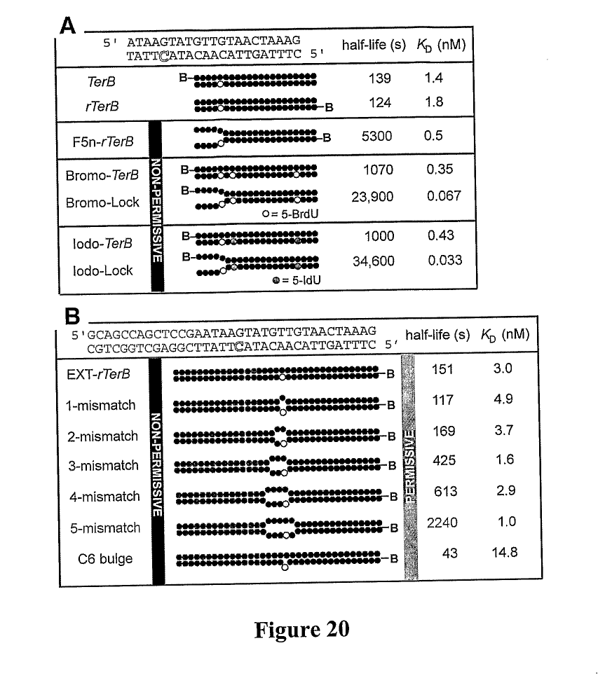

To examine whether the structure of the “locked” species maintains specific contacts, the interaction of Tus with oligonucleotides simultaneously substituted at the T(8) and T(19) positions with IdU or BrdU was examined. These substitutions were observed to have similar effects on the kinetics and thermodynamic parameters describing Tus interactions with both TerB and forked oligonucleotides (FIG. 20A), suggesting that Tus maintains specific contacts with nucleotide bases of TerB at positions between AT(8) and AT(19) when the “lock” forms, and that the structure of the “locked” complex is very similar to that of the wild-type complex in the central region and at the permissive face.

### 5.3.7 C(6) Base Flipping Does Not Explain “Lock” Formation

It was then examined whether flipping of the C(6) base into a site lining the DNA-binding channel of Tus could account for the “locking” behavior. Base flipping should occur readily with TerB oligonucleotides containing just a few unpaired bases around and including C(6), resulting in pronounced stabilization of their complexes with Tus. For these experiments, an extended version of TerB was used to ensure that the mismatched oligonucleotide strands remained hybridized at both ends while bound on the SPR chip. The binding and dissociation kinetics of Tus to wild-type TerB were essentially unaffected by its extension to 37 bp (FIG. 20B). The effects on dissociation rates of introducing mismatches at and around C(6) were modest until the unpaired region extended at least to five base pairs including A-T(3) to A-T(7) of TerB (FIG. 20B). This suggested that although the only site-specific contact required for “lock” formation is with C(6), the presence of restrained regions of double-stranded DNA beyond the limits of the complex is inhibitory. This is inconsistent with a simple base-flipping mechanism. The X-ray structure of the “locked” complex explains these observations.

### 5.3.8 Crystal Structure of the “Tus-Ter Lock”

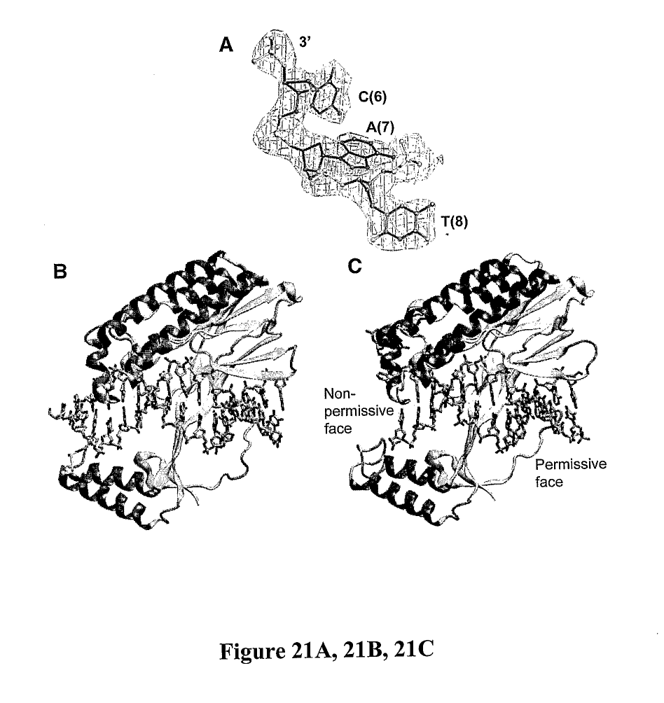

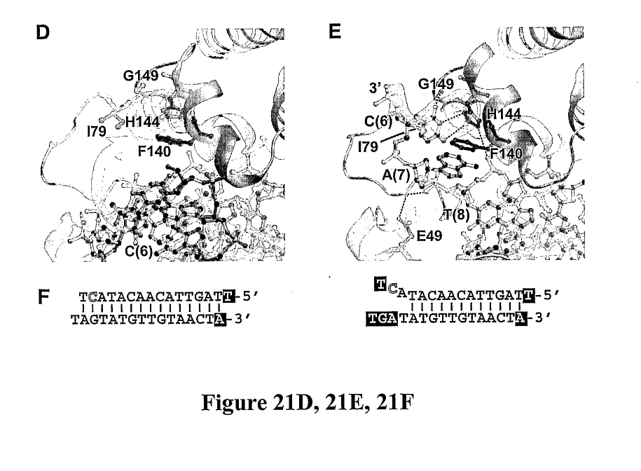

Crystals of Tus in complex with a forked oligonucleotide that resembles the truncated TerA oligonucleotide for the wild-type complex were grown under conditions including sodium iodide in the crystallization buffer. This improved crystallization, and progressive dehydration with increasing concentrations of PEG 3350 also improved the quality of X-ray diffraction patterns. The structure was solved by molecular replacement, using the reported Tus-TerA structure as starting model, to similar resolution (2.7 Å). Data collection and refinement statistics are given in Table 4. The initial model (R_{factor} 43.5, R_{free} 41.23%) was improved by rigid body and positional refinement (R_{factor} 43.07, R_{free} 40.92%). It was clear from the initial 2Fₒ-F_{c} and Fₒ-F_{c} electron density maps that the DNA structure at the non-permissive face of the Tus-Ter complex had been altered and no longer adopted a regular double-stranded structure. The maps revealed new density near His 144, Phe 140 and Gly 149 of Tus (FIG. 21A). Peaks in the Fₒ-F_{c} map of height 7.5 and 5.5 σ corresponded to the C(6) and adjacent A(7) bases. Additional spherical electron density located at crystal-contact positions were interpreted as iodide ions. After four rounds of model building in 0 (Jones et al., (1991) Acta Crystallogr. A47, 110-119) and refinement in REPMAC5 (Murshudov et al., (1997) Acta Crystallogr. D53, 240-255), the R_{factor} and R_{free} were 21.9 and 30.3%, respectively. The final model contained the altered DNA structure, residues 5-309 of Tus, 27 water molecules and 3 iodide ions; coordinates were deposited in the PDB database, with accession code 2EWJ.

TABLE 4
X-Ray Data Collection and Refinement Statistics

| Space group | P4₁2₁2 |
|---|---|
| Unit-cell parameters (Å) | a = 62.7, b = 62.7, c = 251.6 |
| Reflections measured/unique | 46,516/14,359 |
| Resolution range (Å) | 50-2.7 |
| Rsym (%) | 11.2 (52.9) |
| Completeness | 97.9 (98.7) |
| Mean I/σI | 9.3 (1.9) |
| R/R_{free} (%) | 21.9/30.3 |
| rmsd bonds (Å) | 0.017 |
| rmsd angles (°) | 2.07 |

Numbers in parentheses refer to the highest resolution bin 2.8-2.7 Å.

### 5.3.9 Structure of Ter DNA in the “Tus-Ter Lock”

The structure contained all residues on both Ter DNA strands at the permissive end of the “locked” complex except for the unpaired T(20) at the 5′ end and nucleotide A(19) at the 3′ end. Nucleotides in both strands extending from T-A(18) as far as the A-T(8) base pair occupy positions essentially identical to those in the Tus complex with duplex TerA and interact with the same residues in the protein. However, residues in the unpaired region at the non-permissive face either occupy radically different positions or showed no electron density at this resolution. In particular, only the phosphate of T(S), the last residue at the 3′ end, was located. Furthermore, the three unpaired nucleotides at the 5′ end of the other strand could not be detected (FIG. 21F).

Most dramatically, the major differences between the structures of the DNA ligands involve residues that include C(6) at the non-permissive face (FIG. 21). The C(6) base is flipped out of and away from the duplex to bind in a pocket near helix α4 of Tus, centered about 14 Å away from its position in the duplex DNA structure. All three hydrogen-bonding donors/acceptors of the C(6) base form hydrogen bonds with the protein: 02′ with the peptide NH of Gly 149, N3 with the imidazole N^{δ}H of His 144, and the 4-NH₂ group with the peptide carbonyl of Leu 150 (FIGS. 21D and E). The C(6) base ring is otherwise sandwiched in a hydrophobic pocket between the side chains of Ile 79 and Phe 140. In order for C(6) to reach its binding pocket, the T-A(7) base pair of the ligand DNA is also disrupted in the complex, with A(7) also moved out of the helix to stack on the opposite face the phenyl ring of Phe 140. It appears to make no base-specific contacts, consistent with the lack of sequence conservation at this position in known Ter sites (FIG. 6B). That oligonucleotide F6n-rTerB (FIG. 20A), which contains a mispair at position 7 formed a “locked” structure that dissociates at least as slowly as the F5n-rTerB complex is consistent with the observed melting of the T-A(7) base pair in the structure.

### 5.3.10 Structure of Tus in the “Tus-Ter Lock”

The overall structure of Tus in the “locked” complex was similar to that in the previous Tus-TerA complex (FIGS. 21B and C), except for some conformational differences in loops L3 and L4. Residues in the latter, which normally interact with the 5′ strand at the non-permissive face, showed high B-factors and weak electron density, consistent with this region being rather unrestrained by DNA contacts in the “locked” DNA structure. Minor changes also occurred in the orientations of the side chains of residues in α4 that interact directly with C(6), particularly Ile 79, Phe 140 and His 144, but they were generally subtle, suggesting that the cytosine recognition pocket pre-exists on the surface of Tus, awaiting the action of DnaB to liberate the C(6) base from the duplex. The imidazole side chain of His 144 rotated on interaction of its N^{δ}H atom with C(6), bringing N^{e}H close enough to form a new hydrogen bond with the 5′-phosphate group of T(8). It appeared therefore that H is 144 exists as its conjugate acid in the “locked” complex.

### 5.3.11 Progress of the Helicase Leading to “Lock” Formation

The SPR data in FIG. 18 and availability of the two Tus-Ter structures was used to chart the effects, in thermodynamic and kinetic terms, of progressive strand separation on entry of DnaB into the non-permissive end of the Tus-TerB complex. Strand separation as far as A-T(4) (data for oligonucleotides F3n-TerB and F3n-rTerB) resulted in a slight weakening of the Tus-TerB interaction, corresponding to a ˜5-fold increase in K_{D} (ΔΔG ˜0.9 kcal/mol). This was consistent with loss of a single protein-DNA contact near the non-permissive face, which although affecting the strength of the interaction did not change the rate of dissociation of Tus. The lack of strand specificity or effect of deletion of either strand (data for Δ3n-TerB, Δ3n-rTerB) suggested that this represents loss of an electrostatic interaction with the duplex DNA when the strands are separated.

Separation of the next base pair A-T(5) had no further effect on the Tus-TerB interaction. Arg 198 of Tus interacted with A(5) (and also G(6)) in the structure with duplex TerA, but most of its contribution to DNA binding was electrostatic or via interactions with the deoxyribose moieties. This is consistent with there being no strand specificity with the forked DNAs, F4n-TerB and F4n-rTerB (FIG. 18A). The Arg 198 interactions may persist on separation of A-T(5) base pair, but were not significant in the locked structure since complete removal of this strand (top strand in FIG. 18A) had no detectable effect on K_{D} (data for F5n-rTerB cf. F5n-rTerB).

With wild-type TerB, strand-separation to G-C(6) produced the “locked” conformation (F5n-rTerB). Mutagenesis of T(5) to G in the locked oligonucleotide (i.e., F5-TerB(G5)) or its complete removal (in “single O/H C”) increased K_{D} about fourfold (ΔΔG 0.8 kcal/mol). This suggested that there might be some weak specific interaction of Tus with T(5), but it was not apparent in the crystal structure at 2.7 Å resolution. Mutagenesis of the critical C(6) residue to G (in F5n-TerB), A (in F5-TerB(A6)) or T (in F5-TerB(T6)) resulted in a consistent 50-fold increase in K_{D} of the “lock”, indicating that the hydrogen bonds between the C(6) base and its binding residues in α4 of Tus contributed about 2.3 kcal/mol to the free energy of binding.

### 5.3.12 Kinetic Control of “Lock” Formation

The mechanism disclosed herein for fork arrest offers an explanation as to the apparent inefficiency of replication fork arrest at the non-permissive face of Tus-Ter complexes. Although in the SPR experiments Tus dissociated 40-fold more slowly from F5n-rTerB than from rTerB (FIG. 18), the overall difference in K_{D} of the two complexes was only 3 or 4-fold, corresponding to an overall difference in thermodynamic stability (ΔΔG) of less than 0.8 kcal/mol. This implies that formation of the “locked” complex following DnaB-mediated strand separation was a relatively inefficient (i.e., slow) process that is delicately balanced against the rate of further helicase progression and consequent displacement of Tus. This makes sense, in that the search for conformational space by the C(6) base to find its pocket near His 144 of Tus may be relatively inefficient. Any change in local structure of DNA that were to influence the rate of translocation of the helicase into Tus-Ter (e.g, degree of supercoiling) would therefore modulate the efficiency of “lock” formation and consequent fork arrest.

### 5.3.13 Unlocking the “Tus-Ter Lock”

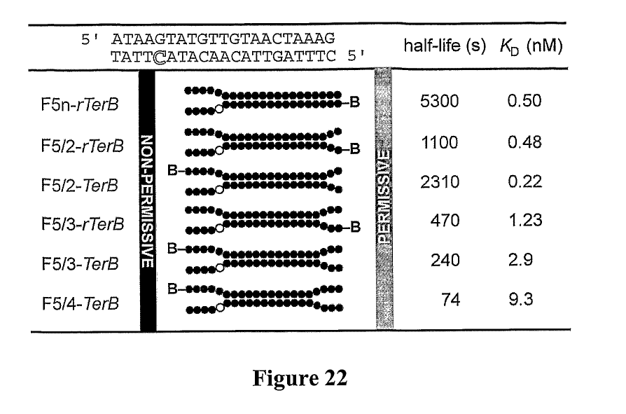

To examine whether strand separation by DnaB at the permissive face was sufficient to force displacement of Tus from the “locked” complex, further SPR experiments were carried out to measure rates of dissociation of Tus from doubly-forked Ter oligonucleotides, with the “lock” sequence at the non-permissive end (FIG. 22). As the forked regions were progressively lengthened at the other (permissive) end, the dissociation rates increased progressively, suggesting that DnaB-mediated strand separation is sufficient even in this context to force dissociation of Tus. There appeared to be no special strand- or nucleotide-specific mechanism for this, suggesting as before that it is the progressive loss of contacts between the duplex DNA and Tus that forces its dissociation, rather than existence of a specific “unlocking” mechanism.

1. A double-stranded oligonucleotide, wherein said oligonucleotide comprises a first strand and a second strand, wherein:
(a) said first strand comprises the sequence:
(SEQ ID NO: 1)
5′-N_{C} R N_{D} G T T G T A A C N_{D} A-3′
or an analogue or derivative of said sequence; and
(b) said second strand comprises the sequence:
(SEQ ID NO: 2)
5′-T N_{D} G T T A C A A C N_{D} T N_{C} C-3′
or an analogue or derivative of said sequence
wherein R is a purine, N_{C} and N_{D} are each a DNA or RNA residue or analogue thereof, N_{D} residues in said first strand and said second strand are sufficiently complementary to permit said N_{D} residues to be annealed in the double-stranded oligonucleotide, and the sequence 5′-GTTGTAAC-3′ (SEQ ID NO: 3) of said first strand is annealed to the complementary sequence 5′-GTTACAAC-3′ (SEQ ID NO: 4) of said second strand.

2. The oligonucleotide of claim 1, wherein said oligonucleotide comprises at least one additional DNA or RNA residue or analogue thereof, at either or both the 5′- and 3′-ends of either or both the first and second strands.

3. The oligonucleotide of either claim 1 or claim 2, wherein said oligonucleotide is forked.

4. The oligonucleotide of any one of claims 1 to 3, wherein:
(a) said first strand comprises the sequence:
(SEQ ID NO: 55)
5′-(N_{A})ₘ N_{E} N_{E} N_{B} N_{B} N_{C} R N_{D} G T T G T A A C N_{D} A
(N_{A})ₙ-3′
or an analogue or derivative of said sequence; and
(b) said second strand comprises the sequence:
(SEQ ID NO: 56)
5′-(N_{A})ₚ T N_{D} G T T A C A A C N_{D} T N_{C} C N_{B} N_{E} N_{E}
(N_{A})ₒ-3′
or an analogue or derivative of said sequence
wherein N_{A}, N_{B} and N_{E} are each any DNA or RNA residue or analogue thereof, each of N_{A} and N_{B} is optional subject to the proviso that when any occurrence of N_{B} is present it is not base-paired to another residue, base-pairing of each of N_{C} to another residue is optional, each of N_{D} is base-paired with another residue, each of N_{E} is optional, subject to the proviso that if one or more of N_{E} is present it is not base-paired unless m=0 or o=0, m, n, o, p, are each an integer including zero, and said first strand and said second strand are of equal or unequal length.

5. The oligonucleotide of any one of claims 1 to 4, wherein said first strand comprises the sequence:
(SEQ ID NO: 57)
5′-(N_{A})₁₋₁₅ N_{E} N_{E} N_{B} N_{B} N_{C} R N_{D} G T T G T A A C N_{D}
A (N_{A})₃-3′
or an analogue or derivative of said sequence.

6. The oligonucleotide of any one of claims 1 to 5, wherein said first strand comprises the sequence:
(SEQ ID NO: 58)
5′-(N_{A})₁₋₁₅ N_{E} N_{E} N_{B} N_{B} N_{C} R T G T T G T A A C T A
A A G-3′
or an analogue or derivative of said sequence.

7. The oligonucleotide of any one of claims 1 to 6, wherein said second strand comprises the sequence:
(SEQ ID NO: 59)
5′-(N_{A})₃ T A G T T A C A A C A T A C N_{B} N_{E} N_{E}
(N_{A})₁₋₁₅-3′
or an analogue or derivative of said sequence.

8. The oligonucleotide of any one of claims 1 to 7, wherein said second strand comprises the sequence:
(SEQ ID NO: 60)
5′-C T T T A G T T A C A A C A T A C N_{B} N_{E} N_{E}
(N_{A})₁₋₁₅-3′
or an analogue or derivative of said sequence.

9. A conjugate, wherein said conjugate comprises the double-stranded oligonucleotide of any one of claims 1 to 8 bound to one or more proteinaceous molecules, nucleic acid molecules, or small molecules.

10. The conjugate of claim 9, wherein said proteinaceous molecule comprises a Tus polypeptide.

11. The conjugate of claim 10, wherein said Tus polypeptide has TerB-binding activity.

12. The conjugate of either claim 10 or claim 11, wherein said Tus polypeptide comprises SEQ ID NO: 5.

13. A conjugate, wherein said conjugate comprises the double-stranded oligonucleotide of any one of claims 1 to 8 bound to:
(i) a Tus polypeptide having TerB-binding activity; and
(ii) a proteinaceous molecule, nucleic acid molecule, or small molecule.

14. Use of the conjugate of any one of claims 9 to 13 for presentation or display of a molecule on a surface.

15. A kit comprising a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce the double-stranded oligonucleotide of any one of claims 1 to 8.

16. A kit for presenting or displaying a first molecule, wherein said first molecule comprises the double-stranded oligonucleotide of any one of claims 1 to 8, in a form suitable for conjugating to:
(a) a second molecule, wherein said second molecule comprises a nucleic acid, polypeptide or small molecule; and
(b) an integer selected from the group consisting of:
(i) a Tus polypeptide in a form suitable for conjugating to another molecule, wherein said double-stranded oligonucleotide and said Tus polypeptide interact in use to present or display another molecule conjugated to said double-stranded oligonucleotide or said polypeptide; and
(ii) mRNA encoding a Tus polypeptide in a form suitable for conjugating to mRNA encoding another polypeptide.

17. A method for presenting or displaying a molecule on a surface, wherein said method comprises contacting a conjugate, wherein said conjugate comprises the double-stranded oligonucleotide of any one of claims 1 to 8 covalently bound to the molecule, with a Tus polypeptide having TerB binding activity bound to the surface, for a time and under conditions sufficient to form a DNA/protein complex, wherein said molecule is displayed on the surface.

18. A method for presenting or displaying a molecule on a surface, wherein said method comprises contacting a conjugate, wherein said conjugate comprises a Tus polypeptide having TerB binding activity covalently bound to the molecule, to the double-stranded oligonucleotide of any one of claims 1 to 8 bound to the surface, for a time and under conditions sufficient to form a DNA/protein complex, wherein the molecule is displayed on the surface.

19. A method for presenting or displaying a molecule, wherein said method comprises:
(i) incubating an mRNA conjugate, wherein said mRNA conjugate comprises mRNA encoding a Tus polypeptide having TerB binding activity fused to mRNA encoding a second polypeptide, for a time and under conditions sufficient for translation of the Tus polypeptide to be produced, and partial or complete translation of the mRNA encoding the second polypeptide to occur, thereby producing a complex comprising the conjugate, a nascent Tus-polypeptide fusion protein encoded by the conjugate and optionally a ribosome;
(ii) incubating the complex with the double-stranded oligonucleotide of any one of claims 1 to 8 for a time and under conditions sufficient to bind to said Tus polypeptide; and
(iii) recovering the complex.

20. A method for the production of a conjugate comprising the double-stranded oligonucleotide of any one of claims 1 to 8 and a peptide, polypeptide or protein, wherein said method comprises:
(i) producing or synthesising said oligonucleotide bound to an agent capable of forming a bond with a peptide, polypeptide or protein; and
(ii) contacting the oligonucleotide with the peptide, polypeptide or protein for a time and under conditions sufficient for a bond to form between the agent and the peptide, polypeptide or protein.

21. A method for the production of a conjugate comprising the double-stranded oligonucleotide of any one of claims 1 to 8 and a Tus polypeptide having Ter-binding activity, wherein said method comprises:
(i) producing or synthesising said oligonucleotide bound to an agent capable of forming a bond with a peptide, polypeptide or protein; and
(ii) contacting the oligonucleotide with the Tus polypeptide for a time and under conditions sufficient for a bond to form between the agent and the peptide, polypeptide or protein.

22. A process for presenting or displaying a molecule, wherein said process comprises:
(i) providing DNA encoding a fusion protein comprising a Tus polypeptide having TerB binding activity fused to a polypeptide of interest;
(ii) transcribing the DNA in the presence of an RNA polymerase to produce an mRNA conjugate comprising mRNA encoding a Tus polypeptide fused to mRNA encoding the polypeptide of interest;
(iii) incubating the mRNA conjugate for a time and under conditions sufficient for translation of a Tus polypeptide to be produced, and partial or complete translation of the mRNA encoding the polypeptide of interest to occur, thereby producing a complex comprising the conjugate, a nascent Tus-fusion protein encoded by the conjugate and optionally a ribosome;
(iv) incubating the complex with the double-stranded oligonucleotide of any one of claims 1 to 8 for a time and under conditions sufficient to bind to said Tus polypeptide; and
(v) recovering the complex.

23. A fusion protein comprising a Tus polypeptide and a peptide, polypeptide or protein of interest for use in the method of claim 19 or the process of claim 22.

24. A polynucleotide encoding the fusion protein of claim 23.

25. A vector containing the polynucleotide of claim 24.

26. A host cell transformed with the vector of claim 25.

27. A chip, wherein said chip comprises the double-stranded oligonucleotide of the first aspect or the conjugate of the second or third aspect.


SEQUENCE LISTING (90 sequences)

SEQ ID NO: 1 - DNA, 13 bp, Artificial Sequence
  synthetic oligonucleotide
nrngttgtaacna

SEQ ID NO: 2 - DNA, 14 bp, Artificial Sequence
  synthetic oligonucleotide
tngttacaacntnc

SEQ ID NO: 3 - DNA, 8 bp, Artificial Sequence
  synthetic oligonucleotide
gttgtaac

SEQ ID NO: 4 - DNA, 8 bp, Artificial Sequence
  synthetic oligonucleotide
gttacaac

SEQ ID NO: 5 - protein, 309 aa, Escherichia coli
MARYDLVDRLNTTFRQMEQELAAFAAHLEQHKLLVARVFSLPEVKKEDEHNPLNRIEVKQHLGNDAQSQALRHFRHLFIQQQSENRSSKAAVRLPGVLCYQVDNLSQAALVSHIQHINKLKTTFEHIVTVESELPTAARFEWVHRHLPGLITLNAYRTLTVLHDPATLRFGWANKHIIKNLHRDEVLAQLEKSLKSPRSVAPWTREEWQRKLEREYQDIAALPQNAKLKIKRPVKVQPIARVWYKGDQKQVQHACPTPLIALINRDNGAGVPDVGELLNYDADNVQHRYKPQAQPLRLIIPRLHLYVAD

SEQ ID NO: 6 - DNA, 23 bp, Artificial Sequence
  synthetic TerB oligonucleotide top strand sequence
aataagtatgttgtaactaaagt

SEQ ID NO: 7 - DNA, 23 bp, Artificial Sequence
  synthetic TerA oligonucleotide top strand sequence
aattagtatgttgtaactaaagt

SEQ ID NO: 8 - DNA, 23 bp, Artificial Sequence
  synthetic TerC oligonucleotide top strand sequence
atataggatgttgtaactaatat

SEQ ID NO: 9 - DNA, 23 bp, Artificial Sequence
  synthetic TerD oligonucleotide top strand sequence
cattagtatgttgtaactaaatg

SEQ ID NO: 10 - DNA, 23 bp, Artificial Sequence
  synthetic TerE oligonucleotide top strand sequence
ttatagtatgttgtaactaagca

SEQ ID NO: 11 - DNA, 23 bp, Artificial Sequence
  synthetic TerF  oligonucleotide top strand sequence
ccttcgtatgttgtaacgacgat

SEQ ID NO: 12 - DNA, 23 bp, Artificial Sequence
  synthetic TerG  oligonucleotide top strand sequence
gtcaaggatgttgtaactaacca

SEQ ID NO: 13 - DNA, 23 bp, Artificial Sequence
  synthetic TerH  oligonucleotide top strand sequence
cgatcgtatgttgtaactatctc

SEQ ID NO: 14 - DNA, 23 bp, Artificial Sequence
  synthetic TerI  oligonucleotide top strand sequence
aacatggaagttgtaactaaccg

SEQ ID NO: 15 - DNA, 23 bp, Artificial Sequence
  synthetic TerJ  oligonucleotide top strand sequence
acgcagaaagttgtaactaatgc

SEQ ID NO: 16 - DNA, 21 bp, Artificial Sequence
  synthetic biotinylated TerB oligonucleotide firststrand sequence
ataagtatgttgtaactaaag

SEQ ID NO: 17 - DNA, 21 bp, Artificial Sequence
  synthetic TerB oligonucleotide second strand sequence
ctttagttacaacatacttat

SEQ ID NO: 18 - DNA, 21 bp, Artificial Sequence
  synthetic rTerB oligonucleotide first strand sequence
ataagtatgttgtaactaaag

SEQ ID NO: 19 - DNA, 21 bp, Artificial Sequence
  synthetic biotinylated rTerB oligonucleotide second strand sequence
ctttagttacaacatacttat

SEQ ID NO: 20 - DNA, 21 bp, Artificial Sequence
  synthetic F2p-rTerB oligonucleotide first strand sequence
ataagtatgttgtaactaacc

SEQ ID NO: 21 - DNA, 21 bp, Artificial Sequence
  synthetic F3p-rTerB oligonucleotide first strand sequence
ataagtatgttgtaactaccc

SEQ ID NO: 22 - DNA, 21 bp, Artificial Sequence
  synthetic F3p-TerB oligonucleotide second strand sequence
gggtagttacaacatacttat

SEQ ID NO: 23 - DNA, 21 bp, Artificial Sequence
  synthetic F4p-rTerB oligonucleotide first strand sequence
ataagtatgttgtaactcccc

SEQ ID NO: 24 - DNA, 21 bp, Artificial Sequence
  synthetic F4p-TerB oligonucleotide second strand sequence
ggggagttacaacatacttat

SEQ ID NO: 25 - DNA, 21 bp, Artificial Sequence
  synthetic F3n-TerB oligonucleotide second strand sequence
ctttagttacaacatactccc

SEQ ID NO: 26 - DNA, 21 bp, Artificial Sequence
  synthetic F3n-rTerB oligonucleotide first strand sequence
gggagtatgttgtaactaaag

SEQ ID NO: 27 - DNA, 21 bp, Artificial Sequence
  synthetic F4n-TerB oligonucleotide second strand sequence
ctttagttacaacataccccc

SEQ ID NO: 28 - DNA, 21 bp, Artificial Sequence
  synthetic F4n-rTerB oligonucleotide first strand sequence
gggggtatgttgtaactaaag

SEQ ID NO: 29 - DNA, 21 bp, Artificial Sequence
  synthetic F5n-TerB oligonucleotide second strand sequence
ctttagttacaacatagcccc

SEQ ID NO: 30 - DNA, 21 bp, Artificial Sequence
  synthetic F5n-rTerB oligonucleotide first strand sequence
ggggctatgttgtaactaaag

SEQ ID NO: 31 - DNA, 16 bp, Artificial Sequence
  synthetic delta5-rTerB oligonucleotide first strand sequence
tatgttgtaactaaag

SEQ ID NO: 32 - DNA, 21 bp, Artificial Sequence
  synthetic F6n-rTerB oligonucleotide first strand sequence
ggggcgatgttgtaactaaag

SEQ ID NO: 33 - DNA, 21 bp, Artificial Sequence
  synthetic F7n-rTerB oligonucleotide first strand sequence
ggggcggtgttgtaactaaag

SEQ ID NO: 34 - DNA, 21 bp, Artificial Sequence
  synthetic biotinylated F5-TerB oligonucleotide first strand sequence
ggggctatgttgtaactaaag

SEQ ID NO: 35 - DNA, 21 bp, Artificial Sequence
  synthetic F5-TerB(G2) oligonucleotide second strand sequence
ctttagttacaacatacttag

SEQ ID NO: 36 - DNA, 21 bp, Artificial Sequence
  synthetic F5-TerB(G3) oligonucleotide second strand sequence
ctttagttacaacatactttt

SEQ ID NO: 37 - DNA, 21 bp, Artificial Sequence
  synthetic F5-TerB(G4) oligonucleotide second strand sequence
ctttagttacaacatactgat

SEQ ID NO: 38 - DNA, 21 bp, Artificial Sequence
  synthetic F5-TerB(G5) oligonucleotide second strand sequence
ctttagttacaacatacgtat

SEQ ID NO: 39 - DNA, 21 bp, Artificial Sequence
  synthetic F5-TerB(C6) oligonucleotide second strand sequence
ctttagttacaacataattat

SEQ ID NO: 40 - DNA, 17 bp, Artificial Sequence
  synthetic delta4p-rTerB oligonucleotide first strand sequence
ataagtatgttgtaact

SEQ ID NO: 41 - DNA, 17 bp, Artificial Sequence
  synthetic delta4p-TerB oligonucleotide second strand sequence
agttacaacatacttat

SEQ ID NO: 42 - DNA, 18 bp, Artificial Sequence
  synthetic delta3n-TerB oligonucleotide second strand sequence
ctttagttacaacatact

SEQ ID NO: 43 - DNA, 18 bp, Artificial Sequence
  synthetic delta3n-rTerB oligonucleotide first strand sequence
agtatgttgtaactaaag

SEQ ID NO: 44 - DNA, 17 bp, Artificial Sequence
  synthetic single O/H C oligonucleotide second strand sequence
ctttagttacaacatac

SEQ ID NO: 45 - DNA, 21 bp, Artificial Sequence
  synthetic Bromo-TerB oligonucleotide second strand sequence
cttnagttacaacanacttat

SEQ ID NO: 46 - DNA, 21 bp, Artificial Sequence
  synthetic Iodo-TerB oligonucleotide second strand sequence
cttnagttacaacanacttat

SEQ ID NO: 47 - DNA, 35 bp, Artificial Sequence
  synthetic Ext-rTerB oligonucleotide first strand sequence
gcagccagctccgaataagtatgttgtaactaaag

SEQ ID NO: 48 - DNA, 35 bp, Artificial Sequence
  synthetic biotinylated Ext-rTerB oligonucleotide second strand sequence
ctttagttacaacatacttattcggagctggctgc

SEQ ID NO: 49 - DNA, 35 bp, Artificial Sequence
  synthetic 1 mismatch oligonucleotide first strand sequence
gcagccagctccgaataattatgttgtaactaaag

SEQ ID NO: 50 - DNA, 35 bp, Artificial Sequence
  synthetic 2 mismatch oligonucleotide first strand sequence
gcagccagctccgaatacttatgttgtaactaaag

SEQ ID NO: 51 - DNA, 35 bp, Artificial Sequence
  synthetic 3 mismatch oligonucleotide first strand sequence
gcagccagctccgaatccttatgttgtaactaaag

SEQ ID NO: 52 - DNA, 35 bp, Artificial Sequence
  synthetic 4 mismatch oligonucleotide first strand sequence
gcagccagctccgaaaccttatgttgtaactaaag

SEQ ID NO: 53 - DNA, 35 bp, Artificial Sequence
  synthetic 5 mismatch oligonucleotide first strand sequence
gcagccagctccgaaacctcatgttgtaactaaag

SEQ ID NO: 54 - DNA, 34 bp, Artificial Sequence
  synthetic flipped C6 oligonucleotide first strand sequence
gcagccagctccgaataatatgttgtaactaaag

SEQ ID NO: 55 - DNA, 19 bp, Artificial Sequence
  synthetic oligonucleotide
nnnnnnrngttgtaacnan

SEQ ID NO: 56 - DNA, 19 bp, Artificial Sequence
  synthetic oligonucleotide
ntngttacaacntncnnnn

SEQ ID NO: 57 - DNA, 21 bp, Artificial Sequence
  synthetic oligonucleotide
nnnnnnrngttgtaacnannn

SEQ ID NO: 58 - DNA, 21 bp, Artificial Sequence
  synthetic oligonucleotide
nnnnnnrtgttgtaactaaag

SEQ ID NO: 59 - DNA, 21 bp, Artificial Sequence
  synthetic oligonucleotide
nnntagttacaacatacnnnn

SEQ ID NO: 60 - DNA, 21 bp, Artificial Sequence
  synthetic oligonucleotide
ctttagttacaacatacnnnn

SEQ ID NO: 61 - DNA, 15 bp, Artificial Sequence
  synthetic oligonucleotide
nnangttgtaacnan

SEQ ID NO: 62 - DNA, 16 bp, Artificial Sequence
  synthetic oligonucleotide
nnnangttgtaacnan

SEQ ID NO: 63 - DNA, 17 bp, Artificial Sequence
  synthetic oligonucleotide
nnnnangttgtaacnan

SEQ ID NO: 64 - DNA, 16 bp, Artificial Sequence
  synthetic oligonucleotide
nnnangttgtaacnan

SEQ ID NO: 65 - DNA, 17 bp, Artificial Sequence
  synthetic oligonucleotide
nnnnangttgtaacnan

SEQ ID NO: 66 - DNA, 18 bp, Artificial Sequence
  synthetic oligonucleotide
nnnnnangttgtaacnan

SEQ ID NO: 67 - DNA, 17 bp, Artificial Sequence
  synthetic oligonucleotide
nnnnangttgtaacnan

SEQ ID NO: 68 - DNA, 18 bp, Artificial Sequence
  synthetic oligonucleotide
nnnnnangttgtaacnan

SEQ ID NO: 69 - DNA, 16 bp, Artificial Sequence
  synthetic oligonucleotide
ntngttacaacntncn

SEQ ID NO: 70 - DNA, 17 bp, Artificial Sequence
  synthetic oligonucleotide
ntngttacaacntncnn

SEQ ID NO: 71 - DNA, 18 bp, Artificial Sequence
  synthetic oligonucleotide
ntngttacaacntncnnn

SEQ ID NO: 72 - DNA, 17 bp, Artificial Sequence
  synthetic oligonucleotide
ntngttacaacntncnn

SEQ ID NO: 73 - DNA, 18 bp, Artificial Sequence
  synthetic oligonucleotide
ntngttacaacntncnnn

SEQ ID NO: 74 - DNA, 19 bp, Artificial Sequence
  synthetic oligonucleotide
ntngttacaacntncnnnn

SEQ ID NO: 75 - DNA, 60 bp, Artificial Sequence
  synthetic oligonucleotide
taatggctggtctgaacgacatcttcgaagctcagaaaatcgaatggcacgaacatatga

SEQ ID NO: 76 - DNA, 62 bp, Artificial Sequence
  synthetic oligonucleotide
cgcgtcatatgttcgtgccattcgattttctgagcttcgaagatgtcgttcagaccagccat

SEQ ID NO: 77 - DNA, 21 bp, Artificial Sequence
  synthetic oligonucleotide
ggggctatgttgtaactaaag

SEQ ID NO: 78 - DNA, 21 bp, Artificial Sequence
  synthetic oligonucleotide
tattcatacaacattgatttc

SEQ ID NO: 79 - DNA, 71 bp, Artificial Sequence
  synthetic oligonucleotide
ggggctatgttgtaactaaagtttttttttttttttttttttttttttttttttttttttttttttttttt

SEQ ID NO: 80 - DNA, 71 bp, Artificial Sequence
  synthetic oligonucleotide
tttttttttttttttttttttttttttttttttttttttttttttttttttattcatacaacattgatttc

SEQ ID NO: 81 - DNA, 16 bp, Artificial Sequence
  synthetic oligonucleotide
ttagttacaacatact

SEQ ID NO: 82 - DNA, 16 bp, Artificial Sequence
  synthetic oligonucleotide
tgatatgttgtaacta

SEQ ID NO: 83 - DNA, 37 bp, Artificial Sequence
  synthetic oligonucleotide
aataagtatgttgtaactaaagtggatcaattcataa

SEQ ID NO: 84 - DNA, 37 bp, Artificial Sequence
  synthetic oligonucleotide
ttatgaattgatccactttagttacaacatacttatt

SEQ ID NO: 85 - DNA, 37 bp, Artificial Sequence
  synthetic oligonucleotide
gggggctatgttgtaactaaagtggatcaattcataa

SEQ ID NO: 86 - DNA, 37 bp, Artificial Sequence; identical to SEQ ID NO: 84
  synthetic oligonucleotide

SEQ ID NO: 87 - DNA, 37 bp, Artificial Sequence
  synthetic oligonucleotide
tatgttgtaactaaagtggatcaattcataaaataag

SEQ ID NO: 88 - DNA, 38 bp, Artificial Sequence
  synthetic oligonucleotide
cttattttatgaattgatccactttagttacaacatac

SEQ ID NO: 89 - DNA, 13 bp, Artificial Sequence
  Synthetic Oligonucleotide
gccgccaccatgg

SEQ ID NO: 90 - protein, 18 aa, Artificial Sequence
  biotin tag sequence
MAGLNDIFEAQKIEWHEH